SEQUENCE LISTING (221 sequences)

SEQ ID NO: 1 - DNA, 47 bp, Artificial
  synthetic
gcactggtgaaatgggagggctaggtggaaggaatctgaggcagtgc

SEQ ID NO: 2 - DNA, 47 bp, Artificial
  synthetic
gcactggtgaaatgggagggctatgtggaaggaatctgaggcagtgc

SEQ ID NO: 3 - DNA, 47 bp, Artificial
  synthetic
gcactgatgaaatgggagggctaggtggaaggaatctgaagcagtgc

SEQ ID NO: 4 - DNA, 47 bp, Artificial
  synthetic
gcactagggaaatgggagggctaggcggaaggaatctgaggtagtgc

SEQ ID NO: 5 - DNA, 47 bp, Artificial
  synthetic
gcactaacgaaatgggagggctaggtggaaggaatctaaggtagtgc

SEQ ID NO: 6 - DNA, 47 bp, Artificial
  synthetic
gcagtggcgaaatgggagggctaggtggaaggaatctgagtcactgc

SEQ ID NO: 7 - DNA, 47 bp, Artificial
  synthetic
gcagtggggaaatgggagggctaggtggaaggaatctgagctactgc

SEQ ID NO: 8 - DNA, 47 bp, Artificial
  synthetic
gcattactgaaatgggagggctaggtggaaggaatctggagtaatgc

SEQ ID NO: 9 - DNA, 47 bp, Artificial
  synthetic
gcgctggggaaatgggagggctaggtggaaggaatctgaggcagtgc

SEQ ID NO: 10 - DNA, 47 bp, Artificial
  synthetic
gcgccagcgaaatgggagggctaggtggaaggaatctgagtcggcgc

SEQ ID NO: 11 - DNA, 45 bp, Artificial
  synthetic
cagtggggaaatgggagggctaggtggaaggaatctgagctactg

SEQ ID NO: 12 - DNA, 45 bp, Artificial
  synthetic
gagtggggaaatgggagggctaggtggaaggaatctgagctactc

SEQ ID NO: 13 - DNA, 43 bp, Artificial
  synthetic
agtggggaaatgggagggctaggtggaaggaatctgagctact

SEQ ID NO: 14 - DNA, 41 bp, Artificial
  synthetic
gtggggaaatgggagggctaggtggaaggaatctgagctac

SEQ ID NO: 15 - DNA, 47 bp, Artificial; identical to SEQ ID NO: 7
  synthetic

SEQ ID NO: 16 - DNA, 47 bp, Artificial; identical to SEQ ID NO: 7
  synthetic

SEQ ID NO: 17 - DNA, 47 bp, Artificial; identical to SEQ ID NO: 7
  synthetic

SEQ ID NO: 18 - DNA, 47 bp, Artificial; identical to SEQ ID NO: 7
  synthetic

SEQ ID NO: 19 - DNA, 47 bp, Artificial; identical to SEQ ID NO: 7
  synthetic

SEQ ID NO: 20 - DNA, 47 bp, Artificial; identical to SEQ ID NO: 7
  synthetic

SEQ ID NO: 21 - DNA, 47 bp, Artificial; identical to SEQ ID NO: 7
  synthetic

SEQ ID NO: 22 - DNA, 47 bp, Artificial; identical to SEQ ID NO: 7
  synthetic

SEQ ID NO: 23 - DNA, 47 bp, Artificial; identical to SEQ ID NO: 7
  synthetic

SEQ ID NO: 24 - DNA, 45 bp, Artificial; identical to SEQ ID NO: 12
  synthetic

SEQ ID NO: 25 - DNA, 43 bp, Artificial; identical to SEQ ID NO: 13
  synthetic

SEQ ID NO: 26 - DNA, 45 bp, Artificial
  synthetic
gggtggggaaatgggagggctaggtggaaggaatctgagctaccc

SEQ ID NO: 27 - DNA, 45 bp, Artificial
  synthetic
gcgtggggaaatgggagggctaggtggaaggaatctgagctacgc

SEQ ID NO: 28 - DNA, 45 bp, Artificial
  synthetic
gggcggggaaatgggagggctaggtggaaggaatctgagctgccc

SEQ ID NO: 29 - DNA, 45 bp, Artificial
  synthetic
gcgcggggaaatgggagggctaggtggaaggaatctgagctgcgc

SEQ ID NO: 30 - DNA, 45 bp, Artificial
  synthetic
gggcggggaaatgggagggctaggtggaaggaatctgagccgccc

SEQ ID NO: 31 - DNA, 45 bp, Artificial
  synthetic
gcgcggggaaatgggagggctaggtggaaggaatctgagccgcgc

SEQ ID NO: 32 - DNA, 45 bp, Artificial
  synthetic
gggccgggaaatgggagggctaggtggaaggaatctgagcggccc

SEQ ID NO: 33 - DNA, 45 bp, Artificial
  synthetic
gcgccgggaaatgggagggctaggtggaaggaatctgagcggcgc

SEQ ID NO: 34 - DNA, 45 bp, Artificial
  synthetic
gagcggggaaatgggagggctaggtggaaggaatctgagccgctc

SEQ ID NO: 35 - DNA, 45 bp, Artificial
  synthetic
gagccgggaaatgggagggctaggtggaaggaatctgagcggctc

SEQ ID NO: 36 - DNA, 45 bp, Artificial
  synthetic
gagtggggaaatgggagggctaggtggaaggaatctgagccactc

SEQ ID NO: 37 - DNA, 45 bp, Artificial
  synthetic
gcgtggggaaatgggagggctaggtggaaggaatctgagccacgc

SEQ ID NO: 38 - DNA, 45 bp, Artificial
  synthetic
gagtcgggaaatgggagggctaggtggaaggaatctgagcgactc

SEQ ID NO: 39 - DNA, 45 bp, Artificial
  synthetic
gcgtcgggaaatgggagggctaggtggaaggaatctgagcgacgc

SEQ ID NO: 40 - DNA, 43 bp, Artificial
  synthetic
ggcggggaaatgggagggctaggtggaaggaatctgagccgcc

SEQ ID NO: 41 - DNA, 43 bp, Artificial
  synthethic
cgcggggaaatgggagggctaggtggaaggaatctgagccgcg

SEQ ID NO: 42 - DNA, 41 bp, Artificial
  synthetic
gcggggaaatgggagggctaggtggaaggaatctgagccgc

SEQ ID NO: 43 - DNA, 39 bp, Artificial
  synthetic
gcgggaaatgggagggctaggtggaaggaatctgagcgc

SEQ ID NO: 44 - DNA, 39 bp, Artificial
  synthetic
cggggaaatgggagggctaggtggaaggaatctgagccg

SEQ ID NO: 45 - DNA, 37 bp, Artificial
  synthetic
ggggaaatgggagggctaggtggaaggaatctgagcc

SEQ ID NO: 46 - DNA, 35 bp, Artificial
  synthetic
gggaaatgggagggctaggtggaaggaatctgagc

SEQ ID NO: 47 - DNA, 45 bp, Artificial; identical to SEQ ID NO: 31
  synthetic

SEQ ID NO: 48 - DNA, 39 bp, Artificial; identical to SEQ ID NO: 43
  synthetic

SEQ ID NO: 49 - DNA, 50 bp, Artificial
  synthetic
cgactcgagaggaaaggttgctaaaggttcggttggattcactcgagtcg

SEQ ID NO: 50 - DNA, 50 bp, Artificial
  synthetic
cgactcgagaggaaaggttggtaaaggttcggttggattcactcgagtcg

SEQ ID NO: 51 - DNA, 50 bp, Artificial
  synthetic
cgactcgagaggaaaggttggtataggttcggttggattcactcgagtcg

SEQ ID NO: 52 - DNA, 50 bp, Artificial
  synthetic
cgactcgagaggaaatgttggtaaaggttcggttggattcactcgagtcg

SEQ ID NO: 53 - DNA, 50 bp, Artificial
  synthetic
cgactcgagaggagaggttggtaaagattcggttggattcactcgagtcg

SEQ ID NO: 54 - DNA, 50 bp, Artificial
  synthetic
cggctcgagaggaaaggttggtaaaggttcggttggattcactcgagtcg

SEQ ID NO: 55 - DNA, 50 bp, Artificial
  synthetic
cgactcgagatgaaaggttggcaaaggttcggttggattcactcgagtcg

SEQ ID NO: 56 - DNA, 48 bp, Artificial
  synthetic
cgagtcgatagaaggtcggtaagtttcggtaggatctgcgacgagacg

SEQ ID NO: 57 - DNA, 48 bp, Artificial
  synthetic
cgagtcgatagaaggttggtaagtttcggttggatctgcgacgagacg

SEQ ID NO: 58 - DNA, 46 bp, Artificial
  synthetic
actcgagaggaaaggttggtaaaggttcggttggattcactcgagt

SEQ ID NO: 59 - DNA, 44 bp, Artificial
  synthetic
gtcgagaggaaaggttggtaaaggttcggttggattcactcgac

SEQ ID NO: 60 - DNA, 42 bp, Artificial
  synthetic
tcgagaggaaaggttggtaaaggttcggttggattcactcga

SEQ ID NO: 61 - DNA, 38 bp, Artificial
  synthetic
gagaggaaaggttggtaaaggttcggttggattcactc

SEQ ID NO: 62 - DNA, 45 bp, Artificial
  synthetic
actcgagaggaaggttggtaaaggttcggttggattcactcgagt

SEQ ID NO: 63 - DNA, 45 bp, Artificial
  synthetic
actcgagaggaaaggttggtaaggttcggttggattcactcgagt

SEQ ID NO: 64 - DNA, 44 bp, Artificial
  synthetic
actcgagaggaaggttggtaaggttcggttggattcactcgagt

SEQ ID NO: 65 - DNA, 46 bp, Artificial; identical to SEQ ID NO: 58
  synthetic

SEQ ID NO: 66 - DNA, 46 bp, Artificial; identical to SEQ ID NO: 58
  synthetic

SEQ ID NO: 67 - DNA, 46 bp, Artificial
  synthetic
actcgagaggaaaggttggtaaaggttcggttggautcactcgagt

SEQ ID NO: 68 - DNA, 46 bp, Artificial; identical to SEQ ID NO: 58
  synthetic

SEQ ID NO: 69 - DNA, 46 bp, Artificial; identical to SEQ ID NO: 67
  synthetic

SEQ ID NO: 70 - DNA, 46 bp, Artificial; identical to SEQ ID NO: 67
  synthetic

SEQ ID NO: 71 - DNA, 46 bp, Artificial; identical to SEQ ID NO: 67
  synthetic

SEQ ID NO: 72 - DNA, 44 bp, Artificial; identical to SEQ ID NO: 59
  synthetic

SEQ ID NO: 73 - DNA, 44 bp, Artificial
  synthetic
ttcgagaggaaaggttggtaaaggttcggttggattcactcgaa

SEQ ID NO: 74 - DNA, 44 bp, Artificial
  synthetic
tgcgagaggaaaggttggtaaaggttcggttggattcactcgca

SEQ ID NO: 75 - DNA, 44 bp, Artificial
  synthetic
ggcgagaggaaaggttggtaaaggttcggttggattcactcgcc

SEQ ID NO: 76 - DNA, 44 bp, Artificial
  synthetic
ggccagaggaaaggttggtaaaggttcggttggattcactggcc

SEQ ID NO: 77 - DNA, 44 bp, Artificial
  synthetic
gcgcagaggaaaggttggtaaaggttcggttggattcactgcgc

SEQ ID NO: 78 - DNA, 44 bp, Artificial
  synthetic
gccgagaggaaaggttggtaaaggttcggttggattcactcggc

SEQ ID NO: 79 - DNA, 44 bp, Artificial
  synthetic
ctcgagaggaaaggttggtaaaggttcggttggattcactcgag

SEQ ID NO: 80 - DNA, 45 bp, Artificial
  synthetic
ctcgagaggaaaggttggtaaaggttcggttggattcactcgagt

SEQ ID NO: 81 - DNA, 44 bp, Artificial
  synthetic
gccgagaggaaaggttggtaaaggttcggttggautcactcggc

SEQ ID NO: 82 - DNA, 44 bp, Artificial; identical to SEQ ID NO: 81
  synthetic

SEQ ID NO: 83 - DNA, 48 bp, Artificial
  synthetic
cggcctagaaggtaggtaagtttcggttggatctacggtcgtaacacg

SEQ ID NO: 84 - DNA, 48 bp, Artificial
  synthetic
cgtcctagaaggtaggtaagtttcggttggatctaggatagtagcacg

SEQ ID NO: 85 - RNA, 46 nt, Artificial
  synthetic
cguguguggguagaugcaccugcgauucgcuaaaaagugccacacg

SEQ ID NO: 86 - RNA, 52 nt, Artificial
  synthetic
cgacguguguggguagaugcaccugcgauucgcuaaaaagugccacacgucg

SEQ ID NO: 87 - RNA, 54 nt, Artificial
  synthetic
cagacguguguggguagaugcaccugcgauucgcuaaaaagugccacacgucug

SEQ ID NO: 88 - DNA, 46 bp, Artificial; identical to SEQ ID NO: 58
  synthetic

SEQ ID NO: 89 - DNA, 46 bp, Artificial; identical to SEQ ID NO: 58
  synthetic

SEQ ID NO: 90 - DNA, 44 bp, Artificial; identical to SEQ ID NO: 81
  synthetic

SEQ ID NO: 91 - DNA, 39 bp, Artificial; identical to SEQ ID NO: 43
  synthetic

SEQ ID NO: 92 - DNA, 39 bp, Artificial; identical to SEQ ID NO: 43
  synthetic

SEQ ID NO: 93 - DNA, 47 bp, Artificial; identical to SEQ ID NO: 7
  synthetic

SEQ ID NO: 94 - DNA, 47 bp, Artificial; identical to SEQ ID NO: 7
  synthetic

SEQ ID NO: 95 - DNA, 47 bp, Artificial; identical to SEQ ID NO: 7
  synthetic

SEQ ID NO: 96 - RNA, 54 nt, Artificial; identical to SEQ ID NO: 87
  synthetic

SEQ ID NO: 97 - DNA, 54 bp, Artificial
  synthetic
cagacgtguguggguagaugcaccugcgauucgcuaaaaagugccacacgucug

SEQ ID NO: 98 - DNA, 54 bp, Artificial
  synthetic
cagacgugugugggtagaugcaccugcgauucgcuaaaaagugccacacgucug

SEQ ID NO: 99 - RNA, 54 nt, Artificial; identical to SEQ ID NO: 87
  synthetic

SEQ ID NO: 100 - DNA, 54 bp, Artificial
  synthetic
cagacguguguggguagatgcaccugcgauucgcuaaaaagugccacacgucug

SEQ ID NO: 101 - RNA, 54 nt, Artificial; identical to SEQ ID NO: 87
  synthetic

SEQ ID NO: 102 - RNA, 54 nt, Artificial; identical to SEQ ID NO: 87
  synthetic

SEQ ID NO: 103 - RNA, 54 nt, Artificial; identical to SEQ ID NO: 87
  synthetic

SEQ ID NO: 104 - RNA, 54 nt, Artificial; identical to SEQ ID NO: 87
  synthetic

SEQ ID NO: 105 - RNA, 54 nt, Artificial; identical to SEQ ID NO: 87
  synthetic

SEQ ID NO: 106 - DNA, 54 bp, Artificial
  synthetic
cagacguguguggguagaugcacctgcgauucgcuaaaaagugccacacgucug

SEQ ID NO: 107 - RNA, 54 nt, Artificial; identical to SEQ ID NO: 87
  synthetic

SEQ ID NO: 108 - RNA, 54 nt, Artificial; identical to SEQ ID NO: 87
  synthetic

SEQ ID NO: 109 - RNA, 54 nt, Artificial; identical to SEQ ID NO: 87
  synthetic

SEQ ID NO: 110 - RNA, 54 nt, Artificial; identical to SEQ ID NO: 87
  synthetic

SEQ ID NO: 111 - DNA, 47 bp, Artificial; identical to SEQ ID NO: 1
  synthetic

SEQ ID NO: 112 - DNA, 47 bp, Artificial; identical to SEQ ID NO: 2
  synthetic

SEQ ID NO: 113 - DNA, 47 bp, Artificial; identical to SEQ ID NO: 3
  synthetic

SEQ ID NO: 114 - DNA, 47 bp, Artificial; identical to SEQ ID NO: 4
  synthetic

SEQ ID NO: 115 - DNA, 47 bp, Artificial; identical to SEQ ID NO: 5
  synthetic

SEQ ID NO: 116 - DNA, 47 bp, Artificial; identical to SEQ ID NO: 6
  synthetic

SEQ ID NO: 117 - DNA, 47 bp, Artificial; identical to SEQ ID NO: 7
  synthetic

SEQ ID NO: 118 - DNA, 47 bp, Artificial; identical to SEQ ID NO: 8
  synthetic

SEQ ID NO: 119 - DNA, 47 bp, Artificial; identical to SEQ ID NO: 9
  synthetic

SEQ ID NO: 120 - DNA, 47 bp, Artificial; identical to SEQ ID NO: 10
  synthetic

SEQ ID NO: 121 - DNA, 45 bp, Artificial; identical to SEQ ID NO: 11
  synthetic

SEQ ID NO: 122 - DNA, 45 bp, Artificial; identical to SEQ ID NO: 12
  synthetic

SEQ ID NO: 123 - DNA, 43 bp, Artificial; identical to SEQ ID NO: 13
  synthetic

SEQ ID NO: 124 - DNA, 41 bp, Artificial; identical to SEQ ID NO: 14
  synthetic

SEQ ID NO: 125 - DNA, 50 bp, Artificial; identical to SEQ ID NO: 49
  synthetic

SEQ ID NO: 126 - DNA, 50 bp, Artificial; identical to SEQ ID NO: 50
  synthetic

SEQ ID NO: 127 - DNA, 50 bp, Artificial; identical to SEQ ID NO: 51
  synthetic

SEQ ID NO: 128 - DNA, 50 bp, Artificial; identical to SEQ ID NO: 52
  synthetic

SEQ ID NO: 129 - DNA, 50 bp, Artificial; identical to SEQ ID NO: 53
  synthetic

SEQ ID NO: 130 - DNA, 50 bp, Artificial; identical to SEQ ID NO: 54
  synthetic

SEQ ID NO: 131 - DNA, 50 bp, Artificial; identical to SEQ ID NO: 55
  synthetic

SEQ ID NO: 132 - DNA, 48 bp, Artificial; identical to SEQ ID NO: 56
  synthetic

SEQ ID NO: 133 - DNA, 48 bp, Artificial; identical to SEQ ID NO: 57
  synthetic

SEQ ID NO: 134 - DNA, 46 bp, Artificial; identical to SEQ ID NO: 58
  synthetic

SEQ ID NO: 135 - DNA, 44 bp, Artificial; identical to SEQ ID NO: 59
  synthetic

SEQ ID NO: 136 - DNA, 42 bp, Artificial; identical to SEQ ID NO: 60
  synthetic

SEQ ID NO: 137 - DNA, 38 bp, Artificial; identical to SEQ ID NO: 61
  synthetic

SEQ ID NO: 138 - DNA, 45 bp, Artificial; identical to SEQ ID NO: 62
  synthetic

SEQ ID NO: 139 - DNA, 45 bp, Artificial; identical to SEQ ID NO: 63
  synthetic

SEQ ID NO: 140 - DNA, 44 bp, Artificial; identical to SEQ ID NO: 64
  synthetic

SEQ ID NO: 141 - DNA, 46 bp, Artificial; identical to SEQ ID NO: 58
  synthetic

SEQ ID NO: 142 - DNA, 44 bp, Artificial; identical to SEQ ID NO: 59
  synthetic

SEQ ID NO: 143 - DNA, 44 bp, Artificial; identical to SEQ ID NO: 73
  synthetic

SEQ ID NO: 144 - DNA, 44 bp, Artificial; identical to SEQ ID NO: 74
  synthetic

SEQ ID NO: 145 - DNA, 44 bp, Artificial; identical to SEQ ID NO: 75
  synthetic

SEQ ID NO: 146 - DNA, 44 bp, Artificial; identical to SEQ ID NO: 76
  synthetic

SEQ ID NO: 147 - DNA, 44 bp, Artificial; identical to SEQ ID NO: 77
  synthetic

SEQ ID NO: 148 - DNA, 44 bp, Artificial; identical to SEQ ID NO: 78
  synthetic

SEQ ID NO: 149 - DNA, 44 bp, Artificial; identical to SEQ ID NO: 79
  synthetic

SEQ ID NO: 150 - DNA, 45 bp, Artificial; identical to SEQ ID NO: 80
  synthetic

SEQ ID NO: 151 - DNA, 48 bp, Artificial; identical to SEQ ID NO: 83
  synthetic

SEQ ID NO: 152 - DNA, 48 bp, Artificial; identical to SEQ ID NO: 84
  synthetic

SEQ ID NO: 153 - RNA, 46 nt, Artificial; identical to SEQ ID NO: 85
  synthetic

SEQ ID NO: 154 - RNA, 54 nt, Artificial; identical to SEQ ID NO: 87
  synthetic

SEQ ID NO: 155 - DNA, 46 bp, Artificial; identical to SEQ ID NO: 58
  synthetic

SEQ ID NO: 156 - DNA, 47 bp, Artificial
  synthetic
actcgagaggaaraggttggtaaaggttcggttggattcactcgagt

SEQ ID NO: 157 - DNA, 44 bp, Artificial; identical to SEQ ID NO: 81
  synthetic

SEQ ID NO: 158 - DNA, 39 bp, Artificial; identical to SEQ ID NO: 43
  synthetic

SEQ ID NO: 159 - DNA, 39 bp, Artificial; identical to SEQ ID NO: 43
  synthetic

SEQ ID NO: 160 - protein, 69 aa, Homo sapiens
RSLQDTEEKSRSFSASQADPLSDPDQMNEDKRHSQGTFTSDYSKYLDSRRAQDFVQWLMNTKRNRNNIA

SEQ ID NO: 161 - protein, 30 aa, Homo sapiens
RSLQDTEEKSRSFSASQADPLSDPDQMNED

SEQ ID NO: 162 - protein, 37 aa, Homo sapiens
HSQGTFTSDYSKYLDSRRAQDFVQWLMNTKRNRNNIA

SEQ ID NO: 163 - protein, 29 aa, Homo sapiens
HSQGTFTSDYSKYLDSRRAQDFVQWLMNT

SEQ ID NO: 164 - protein, 37 aa, Homo sapiens
HDEFERHAEGTFTSDVSSYLEGQAAKEFIAWLVKGRG

SEQ ID NO: 165 - protein, 31 aa, Homo sapiens
HAEGTFTSDVSSYLEGQAAKEFIAWLVKGRG

SEQ ID NO: 166 - protein, 30 aa, Homo sapiens
HAEGTFTSDVSSYLEGQAAKEFIAWLVKGR

SEQ ID NO: 167 - protein, 33 aa, Homo sapiens
HADGSFSDEMNTILDNLAARDFINWLIQTKITD

SEQ ID NO: 168 - protein, 42 aa, Homo sapiens
YAEGTFISDYSIAMDKIHQQDFVNWLLAQKGKKNDWKHNITQ

SEQ ID NO: 169 - protein, 42 aa, Homo sapiens
HADGVFTSDFSKLLGQLSAKKYLESLMGKRVSSNISEDPVPV

SEQ ID NO: 170 - protein, 27 aa, Homo sapiens
HADGVFTSDFSKLLGQLSAKKYLESLM

SEQ ID NO: 171 - protein, 29 aa, Cavia sp.
HSQGTFTSDYSKYLDSRRAQQFLKWLLNV

SEQ ID NO: 172 - protein, 29 aa, Chinchilla sp.
HSQGTFTSDYSKHLDSRYAQEFVQWLMNT

SEQ ID NO: 173 - DNA, 33 bp, Artificial
  synthetic
bnaaatgnganngctakgnggnnggaatctrrr

SEQ ID NO: 174 - DNA, 33 bp, Artificial
  synthetic
bnaaatgnganngctaggnggnnggaatctgar

SEQ ID NO: 175 - DNA, 33 bp, Artificial
  synthetic
tnaaatgnganngctaggnggnnggaatctgag

SEQ ID NO: 176 - DNA, 33 bp, Artificial
  synthetic
tnaaatgnganngctaggnggnnggaatctgaa

SEQ ID NO: 177 - DNA, 33 bp, Artificial
  synthetic
cnaaatgnganngctaggnggnnggaatctgag

SEQ ID NO: 178 - DNA, 33 bp, Artificial
  synthetic
gnaaatgnganngctaggnggnnggaatctgag

SEQ ID NO: 179 - DNA, 33 bp, Artificial
  synthetic
ggaaatgggagggctaggtggaaggaatctgag

SEQ ID NO: 180 - DNA, 32 bp, Artificial
  synthetic
ggaaatgggaggctaggtggaaggaatctgag

SEQ ID NO: 181 - DNA, 33 bp, Artificial; identical to SEQ ID NO: 179
  synthetic

SEQ ID NO: 182 - DNA, 33 bp, Artificial; identical to SEQ ID NO: 179
  synthetic

SEQ ID NO: 183 - DNA, 33 bp, Artificial; identical to SEQ ID NO: 179
  synthetic

SEQ ID NO: 184 - DNA, 33 bp, Artificial; identical to SEQ ID NO: 179
  synthetic

SEQ ID NO: 185 - DNA, 33 bp, Artificial; identical to SEQ ID NO: 179
  synthetic

SEQ ID NO: 186 - DNA, 33 bp, Artificial; identical to SEQ ID NO: 179
  synthetic

SEQ ID NO: 187 - DNA, 33 bp, Artificial; identical to SEQ ID NO: 179
  synthetic

SEQ ID NO: 188 - DNA, 33 bp, Artificial; identical to SEQ ID NO: 179
  synthetic

SEQ ID NO: 189 - DNA, 33 bp, Artificial; identical to SEQ ID NO: 179
  synthetic

SEQ ID NO: 190 - DNA, 33 bp, Artificial; identical to SEQ ID NO: 179
  synthetic

SEQ ID NO: 191 - DNA, 33 bp, Artificial; identical to SEQ ID NO: 179
  synthetic

SEQ ID NO: 192 - DNA, 33 bp, Artificial
  synthetic
bgaaatgggagggctakgyggaaggaatctrrr

SEQ ID NO: 193 - DNA, 33 bp, Artificial
  synthetic
tgaaatgggagggctaggtggaaggaatctgag

SEQ ID NO: 194 - DNA, 33 bp, Artificial
  synthetic
tgaaatgggagggctaggtggaaggaatctgaa

SEQ ID NO: 195 - DNA, 33 bp, Artificial
  synthetic
cgaaatgggagggctaggtggaaggaatctgag

SEQ ID NO: 196 - DNA, 33 bp, Artificial; identical to SEQ ID NO: 179
  synthetic

SEQ ID NO: 197 - DNA, 32 bp, Artificial
  synthetic
akgarnkgttgsyawanrttcgnttggantcn

SEQ ID NO: 198 - DNA, 29 bp, Artificial
  synthetic
agaaggttggtaagtttcggttggatctg

SEQ ID NO: 199 - DNA, 29 bp, Artificial
  synthetic
agaaggtcggtaagtttcggtaggatctg

SEQ ID NO: 200 - DNA, 31 bp, Artificial
  synthetic
aggaaggttggtaaaggttcggttggattca

SEQ ID NO: 201 - DNA, 31 bp, Artificial
  synthetic
aggaaaggttggtaaggttcggttggattca

SEQ ID NO: 202 - DNA, 30 bp, Artificial
  synthetic
aggaaggttggtaaggttcggttggattca

SEQ ID NO: 203 - DNA, 32 bp, Artificial
  synthetic
aggaanggttggtaaangttcgnttggantcn

SEQ ID NO: 204 - DNA, 32 bp, Artificial
  synthetic
aggaaaggttggtaaaggttcggttggattca

SEQ ID NO: 205 - DNA, 32 bp, Artificial; identical to SEQ ID NO: 204
  synthetic

SEQ ID NO: 206 - DNA, 32 bp, Artificial
  synthetic
aggaaaggttggtaaaggttcggttggautca

SEQ ID NO: 207 - DNA, 32 bp, Artificial; identical to SEQ ID NO: 204
  synthetic

SEQ ID NO: 208 - DNA, 32 bp, Artificial
  synthetic
aggaaaggttggtaaaggttcggttggautcg

SEQ ID NO: 209 - DNA, 32 bp, Artificial; identical to SEQ ID NO: 206
  synthetic

SEQ ID NO: 210 - DNA, 32 bp, Artificial; identical to SEQ ID NO: 206
  synthetic

SEQ ID NO: 211 - DNA, 32 bp, Artificial; identical to SEQ ID NO: 206
  synthetic

SEQ ID NO: 212 - DNA, 32 bp, Artificial; identical to SEQ ID NO: 204
  synthetic

SEQ ID NO: 213 - DNA, 10 bp, Artificial
  synthetic
aaggttggta

SEQ ID NO: 214 - DNA, 14 bp, Artificial
  synthetic
aggttcggttggat

SEQ ID NO: 215 - DNA, 14 bp, Artificial
  synthetic
agtttcggttggat

SEQ ID NO: 216 - DNA, 14 bp, Artificial
  synthetic
agtttcggtaggat

SEQ ID NO: 217 - DNA, 14 bp, Artificial
  synthetic
agtttcggtaggat

SEQ ID NO: 218 - DNA, 31 bp, Artificial; identical to SEQ ID NO: 200
  synthetic

SEQ ID NO: 219 - DNA, 31 bp, Artificial; identical to SEQ ID NO: 201
  synthetic

SEQ ID NO: 220 - DNA, 30 bp, Artificial; identical to SEQ ID NO: 202
  synthetic

SEQ ID NO: 221 - DNA, 32 bp, Artificial
  synthetic
akgarakgttgsyawagrttcggttggattca

(19) United States
(12) Patent Application Publication
(10) Pub. No.: US 2015/0232852 A1
(43) Pub. Date: Aug. 20, 2015

(54) Glucagon Binding Nucleic Acids

(71) Applicant: NOXXON PHARMA AG, Berlin (DE)

(72) Inventors: Werner Purschke, Berlin (DE); Simone Sell, Berlin (DE); Axel Vater, Berlin (DE); Klaus Buchner, Berlin (DE); Christian Maasch, Berlin (DE); Sven Klussmann, Berlin (DE)

(21) Appl. No.: 14/351,574

(22) PCT Filed: Oct. 22, 2012
(86) PCT No.: PCT/EP12/04421

§ 371 Date: Apr. 13, 2014

(30) Foreign Application Priority Data
Oct. 21, 2011 (EP) 11 008 467.0
Oct. 21, 2011 (EP) 11 008 473.8
Jan. 10, 2012 (EP) 12 000 107.8
Jan. 10, 2012 (EP) PCT/EP2012/000089

(51) Int. Cl. C12N 15/115; A61K 31/7088

(52) U.S. Cl. CPC C12N 15/115; A61K 31/7088; C12N 2310/16; C12N 2320/30; C12N 2310/351

(57) ABSTRACT

The present invention is related to a nucleic acid molecule capable of binding to a target molecule,
wherein the nucleic acid molecule has a binding affinity to the target molecule,
wherein the binding affinity of the nucleic acid molecule to the target molecule is increased or the same compared to the binding affinity of a reference nucleic acid molecule to the target molecule,
wherein
   a) the nucleic acid molecule comprises a sequence of nucleotides and the reference nucleic acid molecule comprises a sequence of nucleotides, or
   b) the nucleic acid molecule comprises a sequence of nucleotides and at least one modification group and the reference nucleic acid molecule comprises a sequence of nucleotides and the at least one modification group,
wherein the sequence of nucleotides of the nucleic acid molecule and the sequence of nucleotides of the reference nucleic acid molecule are at least partially identical with respect to the nucleobase moiety of the nucleotides but differ with respect to the sugar moiety of the nucleotides,
wherein the sequence of nucleotides of the nucleic acid molecule consists of both ribonucleotides and deoxyribonucleotides and wherein the sequence of nucleotides of the reference nucleic acid molecule consists of either ribonucleotides or deoxyribonucleotides.

The present invention is related to a nucleic acid molecule capable of a binding to glucagon, the use thereof for the manufacture of a medicament, a diagnostic agent, and a detecting agent, respectively, a composition comprising such nucleic acid molecule, a complex comprising such nucleic acid molecule, a method for screening of an antagonist of an activity mediated by glucagon using such nucleic acid molecule, and a method for the detection of such nucleic acid molecule.

Diabetes mellitus (abbr. DM) shows an alarming increase in prevalence worldwide (particularly in Asia), which is mainly driven by type 2 diabetes mellitus (abbr. DM2). Data for the USA show that in 2001 7.9% of persons aged 18 and above were diagnosed with Diabetes compared to 4.9% in 1990. The incidence is linked to both age and body mass index. Mathematical models predict that for a male born in 2000 in the USA the chance to develop Diabetes is 33%, for a female it is even higher at 39%. The same model predicts a loss of 9 life years for these males, and of 12 years for the females. The main risk factors such as obesity, lack of physical activity are well known, but they have been found to be extremely hard to influence. The alarming trends have made the search for new therapeutic agents suitable to treat DM2 even more urgent. Ideal agents should not only reduce blood sugar, but also be at least neutral with respect to body weight and also decrease triglycerides.

Although several anti-hyperglycemic agents are currently available, there is an urgent need for novel agents with different mechanisms of action. Existing agents are often ineffective or become less effective over time and/or are associated with considerable side effects. Two kinds of adverse events are particularly common, disturbing, and potentially harmful: weight gain and hypoglycemia. Exceptions are the agents metformin and acarbose. They are, however, typically only used in early or less severe forms of DM2, have a limited effectiveness, and frequently exert gastrointestinal side effects. In addition, metformin treatment of diabetes is associated with the risk of life-threatening lactic acidosis, particularly in elderly patients with chronic renal and heart failure.

Besides the classical agents, new drugs have entered the market in the last decade. However, most of these are limited by either modest efficacy or side effects that are of particular concern in the target population. The glucagon-like peptide (abbr. GLP-1) analogs (also referred to as incretins) or the inhibitors of the GLP-1-degrading enzyme Dipeptidyl-Peptidase-4 (abbr. DPPIV) were only approved for cases in which other agents have proven to be ineffective and have only shown modest efficacy in terms of anti-hyperglycemic action. The injectable forms of incretins, however, do at least have the advantage of a favorable weight-change profile (Amori, Lau et al. 2007). Therapy with these agents usually requires the injection of long-lasting insulin, to prevent fasting hyperglycemia. Another relatively new substance class, the thiazolidinediones that act as PPAR-agonists, has recently been the subject of discussion concerning their cardiovascular side effects, which has led to a suspension of the marketing authorization in Europe (EMA 2010) and more controlled prescription rules in the US (FDA 2011) for rosiglitazone. This was triggered by an association of rosiglitazone with heart failure, myocardial infarction and death of heart failure (Nissen and Wolski 2007). Another member of the class, troglitazone, had been taken off the market due to drug-induced liver injury. The sale of the third thiazolidinedione, pioglitazone, has been suspended in France after a study suggested the drug (trade name Actos®) raised the risk of bladder cancer (Takeda press release, Jul. 11, 2011).

Whilst the majority of the currently used drugs focus on the relative lack of insulin itself or insulin activity, a lot of research supports the concept that DM2 is at least a bi-hormonal disorder characterized by inadequately high glucagon levels combined with insulin deficiency or insulin resistance (Jiang and Zhang 2003).

Glucagon is a hormone which, like insulin, is produced in the pancreas, but has opposing effects to insulin in peripheral tissue and particularly in the liver. Here it induces mainly gluconeogenesis and glycogenolysis in order to stabilize blood glucose levels between meals.

In the majority of diabetic patients a paradoxical increase of circulating glucagon levels following a mixed meal or carbohydrate ingestion has been reported (Ohneda, Watanabe et al. 1978). This is viewed as a major contributor to increased postprandial blood glucose levels which play an important role in the pathophysiology of micro- and macrovascular complications in DM (Gin and Rigalleau 2000).

Therefore, blocking the action of glucagon by different approaches has been extensively studied. A wealth of peptidyl and non-peptidyl small-molecule glucagon receptor antagonists have been reported (Jiang and Zhang 2003). Some of these small-molecule antagonists, that generally have rather low affinities for the glucagon receptor, have been shown to lower fasting blood glucose or to block exogenous glucagon-stimulated elevation of blood glucose in animal models. A non-peptidyl small molecule glucagon receptor antagonist was shown to block glucagon-induced elevation of hepatic glucose production and blood glucose in humans in a dose-dependent fashion (Petersen and Sullivan 2001). More recently, the reduction of the glucagon receptor expression in db/db-mice by antisense oligonucleotides led to reduction of blood glucose, free fatty acids and triglycerides without development of hypoglycemia (Liang, Osborne et al. 2004). These effects would be ideal for patients with DM2.

Beyond that, glucagon receptor knock-out mice were found to be viable and to show signs of only mild hypoglycemia, improved glucose tolerance and elevated glucagon levels. They are also resistant to diet-induced obesity (Conarello, Jiang et al. 2007), and have a higher insulin sensitivity which may be beneficial in β-cell sparing (Sorensen, Winzell et al. 2006). Moreover, glucagon receptor knock-out mice were resistant to streptozotocin-induced “type 1 diabetes phenotype”, i.e. they showed normoglycemia in the fasted state and after oral and intraperitoneal glucose tolerance tests (Lee, Wang et al. 2011).

Neutralization of glucagon itself by monoclonal antibodies also led to an acute and sustained reduction of blood glucose, triglycerides, HbA1c, and hepatic glucose output (Brand, Rolin et al. 1994; Sorensen, Brand et. al. 2006). However, because of their potential immunogenicity, these and other antibodies might not be a viable option for the long-term treatment of DM.

Essentially, attempts for therapeutic intervention through lowering glucagon levels/activity have yielded a lot of results supporting the concept of glucagon antagonism, but have not lead to compounds with enough potency or to compounds with inacceptable hepatic toxicity.

The hormone gastric inhibitory peptide (abbr. GIP) [, a 42 amino acids long peptide with sequence similarity to glucagon, is released from K-cells predominantly located in the duodenum and proximal jejunum. It is secreted upon nutrient ingestion, especially glucose or fat, with fat being the most potent stimulator of GIP secretion in humans.

The GIP receptor is a typical G-protein coupled receptor with seven transmembrane helices. The GIP receptor gene was found to be expressed in pancreas, stomach, small intestine, adipose tissue, adrenal cortex, pituitary, heart, testis, endothelial cells, bone cells, tracheae, spleen, thymus, lung, kidney, thyroid and several regions in the brain.

GIP does not only induce insulin release as its name suggests, but may also play a role in lipid homeostasis and may be necessary for the development of obesity as shown by several animal studies (Asmar 2011): Daily administration of the GIP receptor antagonist Pro3-GIP for 50 days produced reduced body weight, decreased accumulation of adipose tissue, and marked improvements in levels of glucose, glycated hemoglobin and pancreatic insulin in older high fat fed diabetic mice, together with reduced triglyceride levels in muscle and liver. No change of high-fat diet intake was noted (McClean, Irwin et al. 2007). Pointing in the same direction, GIP receptor knock-out mice were found to be resistant to the development of obesity while wild-type mice fed the same high-fat diet exhibited both hypersecretion of GIP and extreme visceral and subcutaneous fat deposition with insulin resistance (Miyawaki, Yamada et al. 2002). However, the early insulin response after an oral glucose load was impaired, leading to higher blood glucose levels (Miyawaki, Yamada et al. 1999). A detailed description of GIP's contribution to obesity can also be found in a recent review by Irwin and Flatt (Irwin and Flatt 2009).

Other peptides that are sequence-related to glucagon and that are transcribed from the same gene are
glicentin
glicentin-related polypeptide
oxyntomodulin
GLP-1 and its active forms GLP-1(7-36) and GLP-1(7-37)
GLP-2

Furthermore there is the related polypeptide
Prepro vasoactive intestinal peptide(81-122) (Prepro-VIP/intestinal peptide PHV-42)

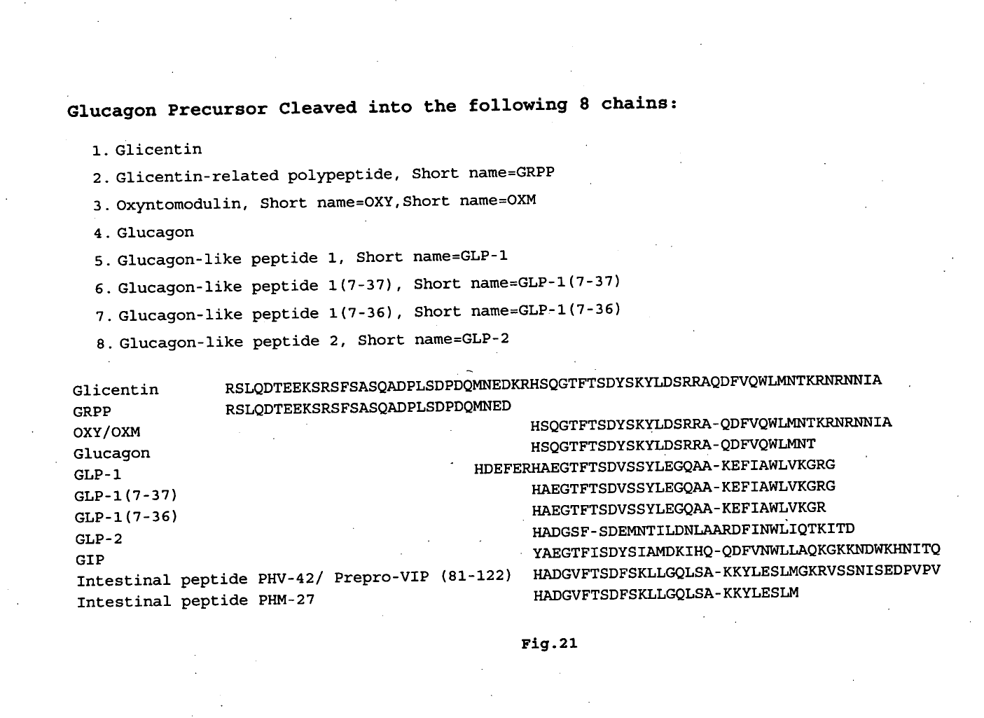

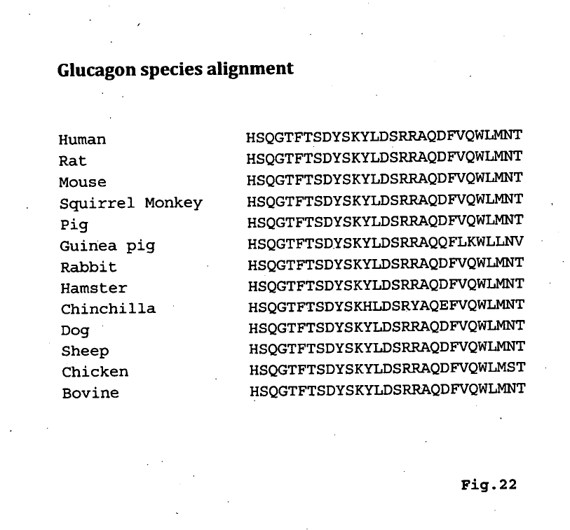

An alignment of the amino acid sequences of these peptides is shown in FIG. 21.

The problem underlying the present invention is to provide a means which specifically interacts with glucagon and/or GIP, whereby the means is suitable for the prevention and/or treatment of diabetes, diabetic complication, diabetic condition and/or hyperglucagonemia.

These and other problems underlying the present invention are solved by the subject matter of the attached independent claims. Preferred embodiments may be taken from the dependent claims.

The problem underlying the present invention is solved in a first aspect which is also the first embodiment of the first aspect by a nucleic acid molecule capable of binding to glucagon, wherein the nucleic acid molecule is selected from the group-comprising a nucleic acid molecule of type A, a nucleic acid molecule of type B and a nucleic acid molecule of type C.

In a second embodiment of the first aspect which is also an embodiment of the first embodiment of the first aspect, the nucleic acid molecule is a nucleic acid molecule of type A, wherein the nucleic acid molecule of type A comprises a central stretch of nucleotides, wherein the central stretch of nucleotides comprises a nucleotide sequence of

[SEQ ID NO: 173]
5′ Bn₁AAATGn₂GAn₃n₄GCTAKGn₅GGn₆n₇GGAATCTRRR 3′,

n₁ is G or rG, n₂ is G or rG, n₃ is G or rG, n₄ is G or rG, n₅ is Y or rT, n₆ is A or rA, n₇ is A or rA, and wherein
any of G, A, T, C, B, K, Y and R is a 2′-deoxyribonucleotide, and
any of rG, rA and rT is a ribonucleotide.

In a third embodiment of the first aspect which is also an embodiment of the second embodiment of the first aspect, the central stretch of nucleotides comprises a nucleotide sequence of

[SEQ ID NO: 174]
5′ Bn₁AAATGn₂GAn₃n₄GCTAGGn₅GGn₆n₇GGAATCTGAR 3′,

n₁ is G or rG, n₂ is G or rG, n₃ is G or rG, n₄ is G or rG, n₅ is T or rT, n₆ is A or rA, n₇ is A or rA, and wherein
any of G, A, T, C, B, and R is a 2′-deoxyribonucleotide, and
any of rG, rA and rT is a ribonucleotide.

In a fourth embodiment of the first aspect which is also an embodiment of the second and the third embodiment of the first aspect, the central stretch of nucleotides comprises a nucleotide sequence selected from the group of

[SEQ ID NO: 175]
5′ Tn₁AAATGn₂GAn₃n₄GCTAGGn₅GGn₆n₇GGAATCTGAG 3′,
[SEQ ID NO: 176]
5′ Tn₁AAATGn₂GAn₃n₄GCTAGGn₅GGn₆n₇GGAATCTGAA 3′,
[SEQ ID NO: 177]
5′ Cn₁AAATGn₂GAn₃n₄GCTAGGn₅GGn₆n₇GGAATCTGAG 3′,
and
[SEQ ID NO: 178]
5′ Gn₁AAATGn₂GAn₃n₄GCTAGGn₅GGn₆n₇GGAATCTGAG 3′,

wherein
n₁ is G or rG, n₂ is G or rG, n₃ is G or rG, n₄ is G or rG, n₅ is T or rT, n₆ is A or rA, n₇ is A or rA, and wherein
any of G, A, T and C is a 2′-deoxyribonucleotide, and
any of rG, rA and rT is a ribonucleotide.

In a fifth embodiment of the first aspect which is also an embodiment of the second, third and fourth embodiment of the first aspect, the central stretch of nucleotides comprises a nucleotide sequence of

[SEQ ID NO: 178]
5′ Gn₁AAATGn₂GAn₃n₄GCTAGGn₅GGn₆n₇GGAATCTGAG 3′,

wherein
n₁ is G or rG, n₂ is G or rG, n₃ is G or rG, n₄ is G or rG, n₅ is T or rT, n₆ is A or rA, n₇ is A or rA, and wherein
any of G, A, T and C, is a 2′-deoxyribonucleotide, and
any of rG, rA and rT is a ribonucleotide.

In a sixth embodiment of the first aspect which is also an embodiment of the second, third and fourth embodiment of the first aspect, the central stretch of nucleotides comprises a nucleotide sequence of

[SEQ ID NO: 177]
5′ Cn₁AAATGn₂GAn₃n₄GCTAGGn₅GGn₆n₇GGAATCTGAG 3′,

wherein
n₁ is G or rG, n₂ is G or rG, n₃ is G or rG, n₄ is G or rG, n₅ is T or rT, n₆ is A or rA, n₇ is A or rA, and
G, A, T and C, are 2′-deoxyribonucleotides, and
rG, rA and rT are ribonucleotides.

In a seventh embodiment of the first aspect which is also an embodiment of the second, third, fourth, fifth and sixth embodiment of the first aspect, the central stretch of nucleotides consists of 2′-deoxyribonucleotides and ribonucleotides.

In an eighth embodiment of the first aspect which is also an embodiment of the second, third, fourth, fifth, sixth and seventh embodiment of the first aspect, the central stretch of nucleotides comprises a nucleotide sequence selected from the group of

[SEQ ID NO: 179]
5′ GrGAAATGGGAGGGCTAGGTGGAAGGAATCTGAG 3′,
[SEQ ID NO: 180]
5′ GGAAATGrGGAGGCTAGGTGGAAGGAATCTGAG 3′,
[SEQ ID NO: 181]
5′ GGAAATGGGArGGGCTAGGTGGAAGGAATCTGAG 3′,
[SEQ ID NO: 182]
5′ GGAAATGGGAGrGGCTAGGTGGAAGGAATCTGAG 3′,
[SEQ ID NO: 183]
5′ GGAAATGGGAGGGCTAGGTGGrAAGGAATCTGAG 3′,
[SEQ ID NO: 184]
5′ GGAAATGGGAGGGCTAGGTGGArAGGAATCTGAG 3′;
[SEQ ID NO: 185]
5′ GGAAATGrGGAGGGCTAGGTGGrAAGGAATCTGAG 3′,
[SEQ ID NO: 186]
5′ GGAAATGGGAGGGCTAGGTGGrArAGGAATCTGAG 3′,
[SEQ ID NO: 187]
5′ GGAAATGrGGAGGGCTAGGTGGrArAGGAATCTGAG 3′,
[SEQ ID NO: 188]
5′ GGAAATGGGArGGGCTAGGTGGrArAGGAATCTGAG 3′,
[SEQ ID NO: 189]
5′ GrGAAATGrGGArGGGCTAGGTGGrArAGGAATCTGAG 3′,
[SEQ ID NO: 190]
5′ GrGAAATGrGGArGrGGCTAGGTGGrArAGGAATCTGAG 3′
and
[SEQ ID NO: 191]
5′ GrGAAATGrGGArGrGGCTAGGrTGGrArAGGAATCTGAG 3′,

wherein
any of G, A, T and C is a 2′-deoxyribonucleotide, and
any of rG, rA and rT is a ribonucleotide.

In a ninth embodiment of the first aspect which is also an embodiment of the second, third, fourth, fifth and sixth embodiment of the first aspect, the central stretch of nucleotides consists of 2′-deoxyribonucleotides.

In a tenth embodiment of the first aspect which is also an embodiment of the second, third, fourth, fifth, sixth, seventh, eighth and ninth embodiment of the first aspect, the nucleic acid molecule comprises in 5′->3′ direction a first terminal stretch of nucleotides, the central stretch of nucleotides and a second terminal stretch of nucleotides, wherein
the first terminal stretch of nucleotides comprises one to seven nucleotides, and
the second terminal stretch of nucleotides comprises one to seven nucleotides.

In an eleventh embodiment of the first aspect which is also an embodiment of the second, third, fourth, fifth, sixth, seventh, eighth and ninth embodiment of the first aspect, the nucleic acid molecule comprises in 5′->3′ direction a second terminal stretch of nucleotides, the central stretch of nucleotides and a first terminal stretch of nucleotides, wherein
the first terminal stretch of nucleotides comprises one to seven nucleotides, and
the second terminal stretch of nucleotides comprises one to seven nucleotides.

In a twelfth embodiment of the first aspect which is also an embodiment of the tenth and eleventh embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′,

wherein
Z₁ is G or absent, Z₂ is S or absent, Z₃ is V or absent, Z₄ is B or absent, Z₅ is B or absent, Z₆ is V or absent, Z₇ is B or absent, Z₈ is V or absent, Z₉ is V or absent, Z₁₀ is B or absent, Z₁₁ is S or absent, and Z₁₂ is C or absent.

In a 13^{th} embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′,

wherein
a) Z₁ is G, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is C, or
b) Z₁ is absent, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is C, or
c) Z₁ is G, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is absent,
   preferably
a) Z₁ is G, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is C, or
b) Z₁ is absent, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is C, or
c) Z₁ is G, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is absent.

In a 14^{th} embodiment of the first aspect which is also an embodiment of the 13^{th} embodiment of the first aspect,
a) the first terminal stretch of nucleotides comprises a nucleotide sequence of 5′ GCACTGG 3′ and the second terminal stretch of nucleotides comprises a nucleotide sequence of 5′ GCAGTGC 3′, or
b) the first terminal stretch of nucleotides comprises a nucleotide sequence of 5′ GCACTGA 3′ and the second terminal stretch of nucleotides comprises a nucleotide sequence of 5′ GCAGTGC 3′, or
c) the first terminal stretch of nucleotides comprises a nucleotide sequence of 5′ GCAGTGG 3′ and the second terminal stretch of nucleotides comprises a nucleotide sequence of 5′ TCACTGC 3′, or
d) the first terminal stretch of nucleotides comprises a nucleotide sequence of 5′ GCACTGG 3′ and the second terminal stretch of nucleotides comprises a nucleotide sequence of 5′ CTACTGC 3′, or
e) the first terminal stretch of nucleotides comprises a nucleotide sequence of 5′ GCGCTGG 3′ and the second terminal stretch of nucleotides comprises a nucleotide sequence of 5′ GCAGTGC 3′, or
f) the first terminal stretch of nucleotides comprises a nucleotide sequence of 5′ GCGCCAG 3′ and the second terminal stretch of nucleotides comprises a nucleotide sequence of 5′ TCGGCGC 3′.

In a 15 embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′,

wherein
a) Z₁ is absent, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is absent, or
b) Z₁ is absent, Z₂ is S, Z₃ is V, Z₄ is B, Z₅ is B, Z₆ is V, Z₇ is B, Z₈ is V, Z₉ is C, Z₁₀ is B, Z₁₁ is absent, and Z₁₂ is absent, or
c) Z₁ is absent, Z₂ is absent, Z₃ is V, Z₄ is B, Z₅ is B, Z₆ is y, Z₇ is B, Z₈ is V, Z₉ is C, Z₁₀ is B, Z₁₁ is S, and Z₁₂ is absent,
   preferably the first terminal stretch of nucleotides comprises a nucleotide sequence of 5′ Z₁Z₂Z₃Z₄Z₅Z₆G 3′ and the second terminal stretch of nucleotides comprises a nucleotide sequence of 5′ CZ₇Z₈Z₉Z₁₀Z₁₁Z₁₂ 3′,
   wherein
a) Z₁ is absent, Z₂ is S, Z₃ is V, Z₄ is G, Z₅ is Y, Z₆ is S, Z₇ is B, Z₈ is R, Z₉ is C, Z₁₀ is B, Z₁₁ is S, and Z₁₂ is absent, or
b) Z₁ is absent, Z₂ is S, Z₃ is V, Z₄ is G, Z₅ is Y, Z₆ is S, Z₇ is B, Z₈ is R, Z₉ is C, Z₁₀ is B, Z₁₁ is absent, and Z₁₂ is absent, or
c) Z₁ is absent, Z₂ is absent, Z₃ is V, Z₄ is G, Z₅ is Y, Z₆ is S, Z₇ is B, Z₈ is R, Z₉ is C, Z₁₀ is B, Z₁₁ is S, and Z₁₂ is absent.

In a 16^{th} embodiment of the first aspect which is also an embodiment of the 15^{th} embodiment of the first aspect,
a) the first terminal stretch of nucleotides comprises a nucleotide sequence of 5′ GCGCGG 3′ and the second terminal stretch of nucleotides comprises a nucleotide sequence of 5′ CTGCGC 3′, or
b) the first terminal stretch of nucleotides comprises a nucleotide sequence of 5′ GCGCGG 3′ and the second terminal stretch of nucleotides comprises a nucleotide sequence of 5′ CCGCGC 3′, or
c) the first terminal stretch of nucleotides comprises a nucleotide sequence of 5′ GGGCCG 3′ and the second terminal stretch of nucleotides comprises a nucleotide sequence of 5′ CGGCCC 3′, or
d) the first terminal stretch of nucleotides comprises a nucleotide sequence of 5′ GCGCCG 3′ and the second terminal stretch of nucleotides comprises a nucleotide sequence of 5′ CGGCGC 3′, or
e) the first terminal stretch of nucleotides comprises a nucleotide sequence of 5′ GAGCGG 3′ and the second terminal stretch of nucleotides comprises a nucleotide sequence of 5′ CCGCTC 3′, or
f) the first terminal stretch of nucleotides comprises a nucleotide sequence of 5′ GCGTGG 3′ and the second terminal stretch of nucleotides comprises a nucleotide sequence of 5′ CCACGC 3′, or
g) the first terminal stretch of nucleotides comprises a nucleotide sequence of 5′ GCGTCG 3′ and the second terminal stretch of nucleotides comprises a nucleotide sequence of 5′ CGACGC 3′.

In a 17^{th} embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′, wherein
a) Z₁ is absent, Z₂ is absent, Z₃ is V, Z₄ is B, Z₅ is B, Z₆ is V, Z₇ is B, Z₈ is V, Z₉ is V, Z₁₀ is B, Z₁₁ is absent, and Z₁₂ is absent, or
b) Z₁ is absent, Z₂ is absent, Z₃ is V, Z₄ is B, Z₅ is B, Z₆ is V, Z₇ is B, Z₈ is V, Z₉ is V, Z₁₀ is absent, Z₁₁ is absent, and Z₁₂ is absent, or
c) Z₁ is absent, Z₂ is absent, Z₃ is absent, Z₄ is B, Z₅ is B, Z₆ is V, Z₇ is B, Z₈ is V, Z₉ is V, Z₁₀ is B, Z₁₁ is absent, and Z₁₂ is absent,
   preferably the first terminal stretch of nucleotides comprises a nucleotide sequence of 5′ Z₁Z₂Z₃Z₄Z₅Z₆G 3′ and the second terminal stretch of nucleotides comprises a nucleotide sequence of 5′ CZ₇Z₈Z₉Z₁₀Z₁₁Z₁₂ 3′,
   wherein
a) Z₁ is absent, Z₂ is absent, Z₃ is V, Z₄ is G, Z₅ is Y, Z₆ is G, Z₇ is Y, Z₈ is R, Z₉ is C, Z₁₀ is B, Z₁₁ is absent, and Z₁₂ is absent, or
b) Z₁ is absent, Z₂ is absent, Z₃ is V, Z₄ is G, Z₅ is Y, Z₆ is G, Z₇ is Y, Z₈ is R, Z₉ is C, Z₁₀ is absent, Z₁₁ is absent, and Z₁₂ is absent, or
c) Z₁ is absent, Z₂ is absent, Z₃ is absent, Z₄ is G, Z₅ is Y, Z₆ is G, Z₇ is Y, Z₈ is R, Z₉ is C, Z₁₀ is B, Z₁₁ is absent, and Z₁₂ is absent.

In an 18^{th} embodiment of the first aspect which is also an embodiment of the 17^{th} embodiment of the first aspect,
a) the first terminal stretch of nucleotides comprises a nucleotide sequence of 5′ GGCGG 3′ and the second terminal stretch of nucleotides comprises a nucleotide sequence of 5′ CCGCC 3′, or
b) the first terminal stretch of nucleotides comprises a nucleotide sequence of 5′ CGCGG 3′ and the second terminal stretch of nucleotides comprises a nucleotide sequence of 5′ CCGCG 3′.

In a 19^{th} embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′, wherein
a) Z₁ is absent, Z₂ is absent, Z₃ is absent, Z₄ is B, Z₅ is B, Z₆ is V, Z₇ is B, Z₈ is V, Z₉ is V, Z₁₀ is absent, Z₁₁ is absent, and Z₁₂ is absent, or
b) Z₁ is absent, Z₂ is absent, Z₃ is absent, Z₄ is B, Z₅ is B, Z₆ is V, Z₇ is B, Z₈ is V, Z₉ is absent, Z₁₀ is absent, Z₁₁ is absent, and Z₁₂ is absent, or
c) Z₁ is absent, Z₂ is absent, Z₃ is absent, Z₄ is B, Z₅ is B, Z₆ is V, Z₇ is B, Z₈ is V, Z₉ is V, Z₁₀ is absent, Z₁₁ is absent, and Z₁₂ is absent,
   preferably the first terminal stretch of nucleotides comprises a nucleotide sequence of 5′ Z₁Z₂Z₃Z₄Z₅Z₆G 3′ and the second terminal stretch of nucleotides comprises a nucleotide sequence of 5′ CZ₇Z₈Z₉Z₁₀Z₁₁Z₁₂ 3′,
   wherein
a) Z₁ is absent, Z₂ is absent, Z₃ is absent, Z₄ is G, Z₅ is Y, Z₆ is G, Z₇ is Y, Z₈ is R, Z₉ is C, Z₁₀ is absent, Z₁₁ is absent, and Z₁₂ is absent, or
b) Z₁ is absent, Z₂ is absent, Z₃ is absent, Z₄ is G, Z₅ is Y, Z₆ is G, Z₇ is Y, Z₈ is R, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is Y, Z₆ is G, Z₇ is Y, Z₈ is R, Z₉ is C, Z₁₀ is absent, Z₁₁ is absent, and Z₁₂ is absent.

In a 20^{th} embodiment of the first aspect which is also an embodiment of the 19^{th} embodiment of the first aspect,
the first terminal stretch of nucleotides comprises a nucleotide sequence of 5′ GCGG 3′ and the second terminal stretch of nucleotides comprises a nucleotide sequence of 5′ CCGC 3′.

In a 21^{st} embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′, wherein
a) Z₁ is absent, Z₂ is absent, Z₃ is absent, Z₄ is absent, Z₅ is B, Z₆ is V, Z₇ is B, Z₈ is V, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is B, Z₆ is V, Z₇ is B, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is V, Z₇ is B, Z₈ is V, Z₉ is absent, Z₁₀ is absent, Z₁₁ is absent, and Z₁₂ is absent,
   preferably the first terminal stretch of nucleotides comprises a nucleotide sequence of 5′ Z₁Z₂Z₃Z₄Z₅Z₆G 3′ and the second terminal stretch of nucleotides comprises a nucleotide sequence of 5′ CZ₇Z₈Z₉Z₁₀Z₁₁Z₁₂ 3′,
   wherein
a) Z₁ is absent, Z₂ is absent, Z₃ is absent, Z₄ is absent, Z₅ is S, Z₆ is S, Z₇ is S, Z₈ is S, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is S, Z₆ is S, Z₇ is S, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is S, Z₇ is S, Z₈ is S, Z₉ is absent, Z₁₀ is absent, Z₁₁ is absent, and Z₁₂ is absent.

In a 22^{nd} embodiment of the first aspect which is also an embodiment of the 21^{st} embodiment of the first aspect,
the first terminal stretch of nucleotides comprises a nucleotide sequence of 5′ GCG 3′ and the second terminal stretch of nucleotides comprises a nucleotide sequence of 5′ CGC 3′.

In a 23^{rd} embodiment of the first aspect which is also an embodiment of the tenth, eleventh and twelfth embodiment of the first aspect, the first terminal stretch of nucleotides comprises a nucleotide sequence of 5′ Z₁Z₂Z₃Z₄Z₅Z₆V 3′ and the second terminal stretch of nucleotides comprises a nucleotide sequence of 5′ BZ₇Z₈Z₉Z₁₀Z₁₁Z₁₂ 3′, wherein
a) Z₁ is absent, Z₂ is absent, Z₃ is absent, Z₄ is absent, Z₅ is absent, Z₆ is V, Z₇ is B, Z₈ is absent, Z₉ is absent, Z₁₀ is absent, Z₁₁ is absent, and Z₁₂ is absent,
b) Z₁ is absent, Z₂ is absent, Z₃ is absent, Z₄ is absent, Z₅ is absent, Z₆ is V, Z₇ is absent, Z₈ is absent, Z₉ is absent, Z₁₀ is absent, Z₁₁ is absent, and Z₁₂ is absent,
c) Z₁ is absent, Z₂ is absent, Z₃ is absent, Z₄ is absent, Z₅ is absent, Z₆ is absent, Z₇ is B, Z₈ is absent, Z₉ is absent, Z₁₀ is absent, Z₁₁ is absent, and Z₁₂ is absent,
d) Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent,
   preferably the first terminal stretch of nucleotides comprises a nucleotide sequence of 5′ Z₁Z₂Z₃Z₄Z₅Z₆G 3′ and the second terminal stretch of nucleotides comprises a nucleotide sequence of 5′ CZ₇Z₈Z₉Z₁₀Z₁₁Z₁₂ 3′,
   wherein
a) Z₁ is absent, Z₂ is absent, Z₃ is absent, Z₄ is absent, Z₅ is absent, Z₆ is G, Z₇ is C, Z₈ is absent, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent.

In a 24^{th} embodiment of the first aspect which is also an embodiment of the second, third, fourth, fifth, sixth, ninth, tenth, eleventh, twelfth, 13^{th}, 14^{th}, 15^{th}, 16^{th}, 17^{th}, 18^{th}, 19^{th}, 20^{th}, 21^{st}, 22^{nd} and 23^{rd} embodiment of the first aspect, the nucleic acid molecule comprises a nucleotide sequence selected from the group of SEQ ID NO: 6 and SEQ ID NO: 7, or

the nucleic acid molecule has an identity of at least 85% to a nucleic acid molecule comprising a nucleotide sequence selected from the group of SEQ ID NO: 6 and SEQ ID NO: 7, or
the nucleic acid molecule is homologous to a nucleic acid molecule comprising a nucleotide sequence selected from the group of SEQ ID NO: 6 and SEQ ID NO: 7, wherein the homology is at least 85%.

In a 25^{th} embodiment of the first aspect which is also an embodiment of the second, third, fourth, fifth, sixth, seventh, eight, tenth, eleventh, twelfth, 13^{th}, 14^{th}, 15^{th}, 16^{th}, 17^{th}, 18^{th}, 19^{th}, 20^{th}, 21^{st}, 22^{nd} and 23^{rd} embodiment of the first aspect, the nucleic acid molecule comprises a nucleotide sequence selected from the group of SEQ ID NO: 23, SEQ ID NO: 43, SEQ ID NO: 47, SEQ ID NO: 48, SEQ ID NO; 91, SEQ ID NO: 92, SEQ ID NO: 158 and SEQ ID NO: 159, or

the nucleic acid molecule has an identity of at least 85% to a nucleic acid molecule comprising a nucleotide sequence according selected from the group of SEQ ID NO: 23, SEQ ID NO: 43, SEQ ID NO: 47, SEQ ID NO: 48, SEQ ID NO: 91, SEQ ID NO: 92, SEQ ID NO: 158 and SEQ ID NO: 159, or
the nucleic acid molecule is homologous to a nucleic acid molecule comprising a nucleotide sequence selected from the group of SEQ ID NO: 43, SEQ ID NO: 47, SEQ ID NO: 48, SEQ ID NO: 91, SEQ ID NO: 92, SEQ ID NO: 158 and SEQ ID NO: 159, wherein the homology is at least 85%.

In a 26^{th} embodiment of the first aspect which is also an embodiment of the first embodiment of the first aspect, the nucleic acid molecule is a nucleic acid molecule of type B, wherein the nucleic acid molecule of type B comprises a central stretch of 29 to 32 nucleotides, wherein the central stretch of nucleotides comprises a nucleotide sequence selected from the group of

[SEQ ID NO: 197]
5′-AKGARn₁KGTTGSYAWAn₂RTTCGn₃TTGGAn₄TCn₅-′3,
[SEQ ID NO: 198]
5′-AGAAGGTTGGTAAGTTTCGGTTGGATCTG-′3,
[SEQ ID NO: 199]
5′-AGAAGGTCGGTAAGTTTCGGTAGGATCTG-′3,
[SEQ ID NO: 200]
5′-AGGAAGGTTGGTAAAGGTTCGGTTGGATTCA-′3,
[SEQ ID NO: 201]
5′-AGGAAAGGTTGGTAAGGTTCGGTTGGATTCA-′3
and
[SEQ ID NO: 202]
5′-AGGAAGGTTGGTAAGGTTCGGTTGGATTCA-′3,

wherein n₁ is A or rA, n₂ is G or rG, n₃ is G or rG, n₄ is T or rU, n₅ is A or rA, and wherein
any of G, A, T, C, K, Y, S, W and R is a 2′-deoxyribonucleotide, and
any of rG, rA and rU is a ribonucleotide.

In a 27^{th} embodiment of the first aspect which is also an embodiment of the 26 embodiment of the first aspect, the central stretch of nucleotides comprises a nucleotide sequence of

[SEQ ID NO: 203]
5′ AGGAAn₁GGTTGGTAAAn₂GTTCGn₃TTGGAn₄TCn₅ 3′,

wherein n₁ is A or rA, n₂ is G or rG, n₃ is G or rG, n₄ is T or rU, n₅ is A or rA, and wherein
any of G, A, T, and C is a 2′-deoxyribonucleotide, and
any of rG, rA and rU is ribonucleotide.

In a 28^{th} embodiment of the first aspect which is also an embodiment of the 26^{th} and 27^{th} embodiment of the first aspect, the central stretch of nucleotides consists of 2′-deoxyribonucleotides and ribonucleotides.

In a 29^{th} embodiment of the first aspect which is also an embodiment of the 26^{th}, 27^{th} and 28^{th} embodiment of the first aspect, the central stretch of nucleotides comprises a nucleotide sequence selected from the group of

[SEQ ID NO: 204]
5′ AGGAArAGGTTGGTAAAGGTTCGGTTGGATTCA 3′,
[SEQ ID NO: 205]
5′ AGGAAAGGTTGGTAAArGGTTCGGTTGGATTCA 3′,
[SEQ ID NO: 206]
5′ AGGAAAGGTTGGTAAAGGTTCGGTTGGArUTCA 3′,
[SEQ ID NO: 207]
5′ AGGAArAGGTTGGTAAArGGTTCGGTTGGATTCA 3′,
[SEQ ID NO: 208]
5′ AGGAArAGGTTGGTAAAGGTTCGGTTGGArUTCG 3′,
[SEQ ID NO: 209]
5′ AGGAArAGGTTGGTAAAGGTTCGGTTGGArUTCA 3′,
[SEQ ID NO: 210]
5′ AGGAArAGGTTGGTAAArGGTTCGGTTGGArUTCA 3′
and
[SEQ ID NO: 211]
5′ AGGAArAGGTTGGTAAArGGTTCGrGTTGGArUTCrA 3′,

wherein any of G, A, T, and C is a 2′-deoxyribonucleotide, and
any of rG, rA and rU is a ribonucleotide.

In a 30^{th} embodiment of the first aspect which is also an embodiment of the 26^{th} and 27^{th} embodiment of the first aspect, the central stretch of nucleotides consists of 2′-deoxyribonucleotides.

In a 31^{st} embodiment of the first aspect which is also an embodiment of the 26^{th}, 27^{th}, 28^{th}, 29^{th} and 30^{th} embodiment of the first aspect, the nucleic acid molecule comprises in 5′->3′ direction a first terminal stretch of nucleotides, the central stretch of nucleotides and a second terminal stretch of nucleotides, wherein
the first terminal stretch of nucleotides comprises three to nine nucleotides, and
the second terminal stretch of nucleotides comprises three to ten nucleotides.

In a 32^{nd} embodiment of the first aspect which is also an embodiment of the 26^{th}, 27^{th}, 28^{th}, 29^{th} and 30^{th} embodiment of the first aspect, the nucleic acid molecule comprises in 5′->3′ direction a second terminal stretch of nucleotides, the central stretch of nucleotides and a first terminal stretch of nucleotides, wherein
the first terminal stretch of nucleotides comprises three to nine nucleotides, and
the second terminal stretch of nucleotides comprises three to ten nucleotides.

In a 33^{rd} embodiment of the first aspect which is also an embodiment of the 31^{st} and 32^{nd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′,

wherein
Z₁ is C or absent, Z₂ is G or absent, Z₃ is R or absent, Z₄ is B or absent, Z₅ is B or absent, Z₆ is S or absent, Z₇ is S or absent, Z₈ is V or absent, Z₉ is V or absent, Z₁₀ is K or absent, Z₁₁ is M or absent, and Z₁₂ is S or absent.

In a 34^{th} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′, wherein
a) Z₁ is C, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is S, or
b) Z₁ is absent, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is S, or
c) Z₁ is C, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is absent,
   preferably the first terminal stretch of nucleotides comprises a nucleotide sequence of 5′ Z₁Z₂Z₃Z₄Z₅Z₆GAG 3′ and the second terminal stretch of nucleotides comprises a nucleotide sequence of 5′ CTCZ₇Z₈Z₉Z₁₀Z₁₁Z₁₂ 3′,
   wherein
a) Z₁ is C, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is G, or
b) Z₁ is absent, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is G, or
c) Z₁ is C, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is absent.

In a 35^{th} embodiment of the first aspect which is also an embodiment of the 34^{th} embodiment of the first aspect,
a) the first terminal stretch of nucleotides comprises a nucleotide sequence of 5′ CGACTCGAG 3′ and the second terminal stretch of nucleotides comprises a nucleotide sequence of 5′ CTCGAGTCG 3′, or
b) the first terminal stretch of nucleotides comprises a nucleotide sequence of 5′ CGGCTCGAG 3′ and the second terminal stretch of nucleotides comprises a nucleotide sequence of 5′ CTCGAGTCG 3′.

In a 36^{th} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′,

wherein
a) Z₁ is absent, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is absent, or
b) Z₁ is absent, Z₂ is G, Z₃ is R, Z₄ is B, Z₅ is B, Z₆ is S, Z₇ is S, Z₈ is V, Z₉ is N, Z₁₀ is K, Z₁₁ is absent, and Z₁₂ is absent, or
c) Z₁ is absent, Z₂ is absent, Z₃ is R, Z₄ is B, Z₅ is B, Z₆ is S, Z₇ is S, Z₈ is V, Z₉ is N, Z₁₀ is K, Z₁₁ is M, and Z₁₂ is absent.

In a 37^{th} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′, wherein
a) Z₁ is absent, Z₂ is absent, Z₃ is R, Z₄ is B, Z₅ is B, Z₆ is S, Z₇ is S, Z₈ is V, Z₉ is N, Z₁₀ is K, Z₁₁ is absent, and Z₁₂ is absent, or
b) Z₁ is absent, Z₂ is absent, Z₃ is R, Z₄ is B, Z₅ is B, Z₆ is S, Z₇ is S, Z₈ is V, Z₉ is N, Z₁₀ is absent, Z₁₁ is absent, and Z₁₂ is absent, or
c) Z₁ is absent, Z₂ is absent, Z₃ is absent, Z₄ is B, Z₅ is B, Z₆ is S, Z₇ is S, Z₈ is V, Z₉ is N, Z₁₀ is K, Z₁₁ is absent, and Z₁₂ is absent,
   preferably the first terminal stretch of nucleotides comprises a nucleotide sequence of 5′ Z₁Z₂Z₃Z₄Z₅Z₆GAG 3′ and the second terminal stretch of nucleotides comprises a nucleotide sequence of 5′ CTCZ₇Z₈Z₉Z₁₀Z₁₁Z₁₂ 3′, wherein
a) Z₁ is absent, Z₂ is absent, Z₃ is A, Z₄ is C, Z₅ is T, Z₆ is C, Z₇ is G, Z₈ is A, Z₉ is G, Z₁₀ is T, Z₁₁ is absent, and Z₁₂ is absent, or
b) Z₁ is absent, Z₂ is absent, Z₃ is A, Z₄ is C, Z₅ is T, Z₆ is C, Z₇ is G, Z₈ is A, Z₉ is G, Z₁₀ is absent, Z₁₁ is absent, and Z₁₂ is absent, or
c) Z₁ is absent, Z₂ is absent, Z₃ is absent, Z₄ is C, Z₅ is T, Z₆ is C, Z₇ is G, Z₈ is A, Z₉ is G, Z₁₀ is T, Z₁₁ is absent, and Z₁₂ is absent,
preferably Z₁ is absent, Z₂ is absent, Z₃ is A, Z₄ is C, Z₅ is T, Z₆ is C, Z₇ is G, Z₈ is A, Z₉ is G, Z₁₀ is T, Z₁₁ is absent, and Z₁₂ is absent.

In a 38^{th} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′,

wherein
a) Z₁ is absent, Z₂ is absent, Z₃ is absent, Z₄ is B, Z₅ is B, Z₆ is S, Z₇ is S, Z₈ is V, Z₉ is N, Z₁₀ is absent, Z₁₁ is absent, and Z₁₂ is absent, or
b) Z₁ is absent, Z₂ is absent, Z₃ is absent, Z₄ is absent, Z₅ is B, Z₆ is S, Z₇ is S, Z₈ is V, Z₉ is N, Z₁₀ is absent, Z₁₁ is absent, and Z₁₂ is absent, or
c) Z₁ is absent, Z₂ is absent, Z₃ is absent, Z₄ is B, Z₅ is B, Z₆ is S, Z₇ is S, Z₈ is V, Z₉ is absent, Z₁₀ is absent, Z₁₁ is absent, and Z₁₂ is absent,
   preferably the first terminal stretch of nucleotides comprises a nucleotide sequence of 5′ Z₁Z₂Z₃Z₄Z₅Z₆SAG 3′ and the second terminal stretch of nucleotides comprises a nucleotide sequence of 5′ CTSZ₇Z₈Z₉Z₁₀Z₁₁Z₁₂ 3′,
   wherein
a) Z₁ is absent, Z₂ is absent, Z₃ is absent, Z₄ is B, Z₅ is B, Z₆ is S, Z₇ is S, Z₈ is S, Z₉ is V, Z₁₀ is absent, Z₁₁ is absent, and Z₁₂ is absent, or
b) Z₁ is absent, Z₂ is absent, Z₃ is absent, Z₄ is absent, Z₅ is B, Z₆ is S, Z₇ is S, Z₈ is S, Z₉ is V, Z₁₀ is absent, Z₁₁ is absent, and Z₁₂ is absent, or
c) Z₁ is absent, Z₂ is absent, Z₃ is absent, Z₄ is B, Z₅ is B, Z₆ is S, Z₇ is S, Z₈ is S, Z₉ is absent, Z₁₀ is absent, Z₁₁ is absent, and Z₁₂ is absent.

In a 39^{th} embodiment of the first aspect which is also an embodiment of the 38^{th} embodiment of the first aspect,
a) the first terminal stretch of nucleotides comprises a nucleotide sequence of 5′ GTCGAG 3′ and the second terminal stretch of nucleotides comprises a nucleotide sequence of 5′ CTCGAC 3′, or
b) the first terminal stretch of nucleotides comprises a nucleotide sequence of 5′ TGCGAG 3′ and the second terminal stretch of nucleotides comprises a nucleotide sequence of 5′ CTCGCA 3′, or
c) the first terminal stretch of nucleotides comprises a nucleotide sequence of 5′ GGCCAG 3′ and the second terminal stretch of nucleotides comprises a nucleotide sequence of 5′ CTGGCC 3′, or
d) the first terminal stretch of nucleotides comprises a nucleotide sequence of 5′ GCCGAG 3′ and the second terminal stretch of nucleotides comprises a nucleotide sequence of 5′ CTCGGC 3′, or
e) the first terminal stretch of nucleotides comprises a nucleotide sequence of 5′ CTCGAG 3′ and the second terminal stretch of nucleotides comprises a nucleotide sequence of 5′ CTCGAG 3′.

In a 40^{th} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′, wherein
a) Z₁ is absent, Z₂ is absent, Z₃ is absent, Z₄ is absent, Z₅ is B, Z₆ is S, Z₇ is S, Z₈ is V, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is B, Z₆ is S, Z₇ is S, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is S, Z₇ is S, Z₈ is V, Z₉ is absent, Z₁₀ is absent, Z₁₁ is absent, and Z₁₂ is absent,
   wherein preferably the first terminal stretch of nucleotides comprises a nucleotide sequence of 5′ Z₁Z₂Z₃Z₄Z₅Z₆GAG 3′ and the second terminal stretch of nucleotides comprises a nucleotide sequence of 5′ CTCZ₇Z₈Z₉Z₁₀Z₁₁Z₁₂ 3′,
   wherein
a) Z₁ is absent, Z₂ is absent, Z₃ is absent, Z₄ is absent, Z₅ is T, Z₆ is C, Z₇ is G, Z₈ is A, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is T, Z₆ is C, Z₇ is G, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is C, Z₇ is G, Z₈ is A, Z₉ is absent, Z₁₀ is absent, Z₁₁ is absent, and Z₁₂ is absent.

In a 41^{st} embodiment of the first aspect which is also an embodiment of the 31^{st}, 32^{nd} and 33^{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′,

wherein
a) Z₁ is absent, Z₂ is absent, Z₃ is absent, Z₄ is absent, Z₅ is absent, Z₆ is S, Z₇ is S, Z₈ is absent, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is absent, Z₆ is absent, Z₇ is S, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is S, Z₇ is absent, Z₈ is absent, Z₉ is absent, Z₁₀ is absent, Z₁₁ is absent, and Z₁₂ is absent.

In a 42^{nd} embodiment of the first aspect which is also an embodiment of the 31^{st}, 32^{nd} and 33_{rd} embodiment of the first aspect, the first terminal stretch of nucleotides comprises a nucleotide sequence of 5′ Z₁Z₂Z₃Z₄Z₅Z₆SAK 3′ and the second terminal stretch of nucleotides comprises a nucleotide sequence of 5′ CKVZ₇Z₈Z₉Z₁₀Z₁₁Z₁₂ 3′,

wherein Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent, or wherein the first terminal stretch of nucleotides comprises a nucleotide sequence of 5′ Z₁Z₂Z₃Z₄Z₅Z₆GAG 3′ and the second terminal stretch of nucleotides comprises a nucleotide sequence of 5′ CTCZ₇Z₈Z₉Z₁₀Z₁₁Z₂ 3′,
wherein Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent.

In a 43^{rd} embodiment of the first aspect which is also an embodiment of the 26^{th}, 27^{th}, 28^{th}, 30^{th}, 31^{st}, 32^{nd}, 33^{rd}, 34^{th}, 35^{th}, 36^{th}, 37^{th}, 38^{th}, 39^{th}, 40^{th}, 41^{st} and 42^{nd} embodiment of the first aspect, the nucleic acid molecule comprises a nucleotide sequence selected from the group of SEQ ID NO: 50, SEQ ID NO: 54, SEQ ID NO: 58, SEQ ID NO: 59, SEQ ID NO: 88 and SEQ ID NO; 155, or

the nucleic acid molecule has an identity of at least 85% to a nucleic acid molecule comprising a nucleotide sequence selected from the group of SEQ ID NO: 50, SEQ ID NO: 54, SEQ ID NO: 58, SEQ ID NO: 59, SEQ ID NO: 88 and SEQ ID NO: 155, or
the nucleic acid molecule is homologous to a nucleic acid molecule comprising a nucleotide sequence selected from the group of SEQ ID NO: 50, SEQ ID NO: 54, SEQ ID NO: 58, SEQ ID NO: 59, SEQ ID NO: 88 and SEQ ID NO: 155, wherein the homology is at least 85%.

In a 44^{th} embodiment of the first aspect which is also an embodiment of the 26^{th}, 27^{th}, 28^{th}, 29^{th}, 31^{st}, 32^{nd}, 33^{rd}, 34^{th}, 35^{th}, 36^{th}, 37^{th}, 38^{th}, 39^{th}, 40^{th}, 41^{st} and 42^{nd} embodiment of the first aspect, the nucleic acid molecule comprises a nucleotide sequence selected from the group of SEQ ID NO: 71, SEQ ID NO: 81, SEQ ID NO: 82, SEQ ID NO: 89, SEQ ID NO: 90, SEQ ID NO: 156 and SEQ ID NO: 157, or

the nucleic acid molecule has an identity of at least 85% to a nucleic acid molecule comprising a nucleotide sequence selected from the group of SEQ ID NO: 71, SEQ ID NO: 81, SEQ ID NO: 82, SEQ ID NO: 89, SEQ ID NO: 90, SEQ ID NO: 156 and SEQ ID NO: 157, or
the nucleic acid molecule is homologous to a nucleic acid molecule comprising a nucleotide sequence selected from the group of SEQ ID NO: 71, SEQ ID NO: 81, SEQ ID NO: 82, SEQ ID NO: 89, SEQ ID NO: 90, SEQ ID NO: 156 and SEQ ID NO: 157, wherein the homology is at least 85%.

In a 45^{th} embodiment of the first aspect which is also an embodiment of the first embodiment of the first aspect, the nucleic acid molecule is a nucleic acid molecule of type C,

wherein the nucleic acid molecule of type C comprises a nucleotide sequence selected from the group of SEQ ID NO: 83; SEQ ID NO: 84, SEQ ID NO: 85, SEQ ID NO: 86, SEQ ID NO: 87, SEQ ID NO: 97 and SEQ ID NO: 102, or
wherein the nucleic acid molecule has an identity of at least 85% to the nucleic acid molecule comprising a nucleotide sequence selected from the group of SEQ ID NO: 83; SEQ ID NO: 84, SEQ ID NO: 85, SEQ ID NO: 86, SEQ ID NO: 87, SEQ ID NO: 97 and SEQ ID NO: 102, or
wherein the nucleic acid molecule is homologous to a nucleic acid molecule comprising a nucleotide sequence selected from the group of SEQ ID NO: 83; SEQ ID NO: 84, SEQ ID NO: 85, SEQ ID NO: 86, SEQ ID NO: 87, SEQ ID NO: 97 and SEQ ID NO: 102 wherein the homology is at least 85%.

In a 46^{th} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23_{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th} and 45^{th} embodiment of the first aspect, the nucleotides of or the nucleotides forming the nucleic acid molecule are L-nucleotides.

In a 47^{th} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23_{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th} and 45^{th} embodiment of the first aspect, the nucleic acid molecule is an L-nucleic acid molecule.

In a 48^{th} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th} and 47^{th} embodiment of the first aspect, the nucleic acid molecule comprises at least one binding moiety which is capable of binding glucagon, wherein such binding moiety consists of L-nucleotides.

In a 49^{th} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th} and 48^{th} embodiment of the first aspect, the nucleic acid molecule is an antagonist of an activity mediated by glucagon.

In a 50^{th} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th} and 49^{th} embodiment of the first aspect, the nucleic acid molecule is capable of binding to GIP.

In a 51st embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th} and 50^{th} embodiment of the first aspect, the nucleic acid is an antagonist of an activity mediated by GIP.

In a 52^{nd} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th} and 51^{st} embodiment of the first aspect, the nucleic acid molecule comprises a modification group, wherein excretion rate of the nucleic acid molecule comprising the modification group from an organism is decreased compared to a nucleic acid not comprising the modification group.

In a 53^{rd} embodiment of the first aspect which is also an embodiment of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th} and 51^{st} embodiment of the first aspect, the nucleic acid molecule comprises a modification group, wherein the nucleic acid molecule comprising the modification group has an increased retention time in an organism compared to a nucleic acid molecule not comprising the modification group.

In a 54^{th} embodiment of the first aspect which is also an embodiment of the 52^{nd} and 53^{rd} embodiment of the first aspect, the modification group is selected from the group comprising biodegradable and non-biodegradable modifications, preferably the modification group is selected from the group comprising polyethylene glycol, linear polyethylene glycol, branched polyethylene glycol, hydroxyethyl starch, a peptide, a protein, a polysaccharide, a sterol, polyoxypropylene, polyoxyamidate and poly(2-hydroxyethyl)-L-glutamine.

In a 55^{th} embodiment of the first aspect which is also an embodiment of the 54^{th} embodiment of the first aspect, the modification group is a polyethylene glycol consisting of a linear polyethylene glycol or branched polyethylene glycol, wherein the molecular weight of the polyethylene glycol is preferably from about 20,000 to about 120,000 Da, more preferably from about 30,000 to about 80,000 Da and most preferably about 40,000 Da.

In a 56^{th} embodiment of the first aspect which is also an embodiment of the 54^{th} embodiment of the first aspect, the modification group is hydroxyethyl starch, wherein the molecular weight of the hydroxyethyl starch is from about 50 kDa to about 1000 kDa, more preferably from about 100 kDa to about 700 kDa and most preferably from 300 kDa to 500 kDa.

In a 57^{th} embodiment of the first aspect which is also an embodiment of the 52^{nd}, 53^{rd}, 54^{th}, 55^{th} and 56^{th} embodiment of the first aspect, the modification group is coupled to the nucleic acid molecule via a linker, wherein preferably the linker is a biodegradable linker.

In a 58^{th} embodiment of the first aspect which is also an embodiment of the 52^{nd}, 53^{rd}, 54^{th}, 55^{th} and 56^{th} embodiment of the first aspect, the modification group is coupled to the 5′-terminal nucleotide and/or the 3′-terminal nucleotide of the nucleic acid molecule and/or to a nucleotide of the nucleic acid molecule between the 5′-terminal nucleotide of the nucleic acid molecule and the 3′-terminal nucleotide of the nucleic acid molecule.

In a 59^{th} embodiment of the first aspect which is also an embodiment of the 52^{nd}, 53^{rd}, 54^{th}, 55^{th}, 56^{th}, 57^{th} and 58^{th} embodiment of the first aspect, the organism is an animal or a human body, preferably a human body.

The problem underlying the present invention is solved in a second aspect which is also the first embodiment of the second aspect by a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect, for use in a method for the treatment and/or prevention of a disease or disorder or hyperglucagonemia.

In a second embodiment of the second aspect which is also an embodiment of the first embodiment of the second aspect, the disease or disorder is selected from the group comprising diabetes, diabetic complication and diabetic condition.

In a third embodiment of the second aspect which is also an embodiment of the second embodiment of the second aspect, the diabetes is selected from the group comprising type 1 diabetes, type 2 diabetes and gestational diabetes.

In a fourth embodiment of the second aspect which is also an embodiment of the third embodiment of the second aspect, the diabetic complication or diabetic condition is a diabetic complication or a diabetic condition selected from the group of atherosclerosis, coronary artery disease, diabetic foot disease, diabetic retinopathy, proliferative diabetic retinopathy, diabetic macular edema, diabetic vitreoretinopathy, proliferative diabetic vitreoretinopathy, diabetic nephropathy, diabetic neuropathy, glucose intolerance, heart disease, high blood pressure, high cholesterol, impaired glucose tolerance, impotence, insulin resistance, kidney failure, metabolic syndrome, non-alcoholic fatty liver disease, non-alcoholic steatohepatitis with or without fibrosis, peripheral vascular disease, reduced glucose sensitivity, reduced insulin sensitivity, obesity, hepatic steatosis, hyperglycaemia, diabetes-associated vascular inflammation, diabetic ketoacidosis, hyperosmolar hyperglycemic non-ketoic coma, weight loss necrolytic migratory erythema, anemia, venous thrombosis in the present of normal coagulation function and neuropsychiatric manifestations.

The problem underlying the present invention is solved in a third aspect which is also the first embodiment of the third aspect by a pharmaceutical composition comprising a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect and optionally a further constituent, wherein the further constituent is selected from the group comprising pharmaceutically acceptable excipients, pharmaceutically acceptable carriers and pharmaceutically active agents.

In a second embodiment of the third aspect which is also an embodiment of the first embodiment of the third aspect the pharmaceutical composition comprises a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect and a pharmaceutically acceptable carrier.

The problem underlying the present invention is solved in a fourth aspect which is also the first embodiment of the fourth aspect by the use of a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{rd}, 53^{nd}, 54^{th}, 55^{th}, 56^{th}, 57^{th}, 58^{th} and 59^{th} embodiment of the first aspect for the manufacture of a medicament.

In a second embodiment of the fourth aspect which is also an embodiment of the first embodiment of the fourth aspect, the medicament is for use in human medicine or for use in veterinary medicine.

The problem underlying the present invention is solved in a fifth aspect which is also the first embodiment of the fifth aspect by the use of a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect for the manufacture of a diagnostic means.

In a third embodiment of the fourth aspect which is also an embodiment of the first embodiment of the fourth aspect, the medicament is for the treatment and/or prevention of a disease or disorder or hyperglucagonemia, wherein the disease or disorder is selected from the group diabetes, diabetic complication, and diabetic condition.

In a fourth embodiment of the fourth aspect which is also an embodiment of the third embodiment of the fourth aspect, the diabetes is selected from the group type 1 diabetes, type 2 diabetes and gestational diabetes.

In a fifth embodiment of the fourth aspect which is also an embodiment of the third embodiment of the fourth aspect, the diabetic complication or diabetic condition is a diabetic complication or a diabetic condition selected from the group of atherosclerosis, coronary artery disease, diabetic foot disease, diabetic retinopathy, proliferative diabetic retinopathy, diabetic macular edema, diabetic vitreoretinopathy, proliferative diabetic vitreoretinopathy, diabetic nephropathy, diabetic neuropathy, glucose intolerance, heart disease, high blood pressure, high cholesterol, impaired glucose tolerance, impotence, insulin resistance, kidney failure, metabolic syndrome, non-alcoholic fatty liver disease, non-alcoholic steatohepatitis with or without fibrosis, peripheral vascular disease, reduced glucose sensitivity, reduced insulin sensitivity, obesity, hepatic steatosis, hyperglycaemia, diabetes-associated vascular inflammation, diabetic ketoacidosis, hyperosmolar hyperglycemic non-ketoic coma, weight loss necrolytic migratory erythema, anemia, venous thrombosis in the present of normal coagulation function and neuropsychiatric manifestations.

The problem underlying the present invention is solved in a sixth aspect which is also the first embodiment of the sixth aspect by a complex comprising a nucleic acid molecule according to any one of claims 1 to 59 and glucagon and/or GIP, wherein preferably the complex is a crystalline complex.

The problem underlying the present invention is solved in a seventh aspect which is also the first embodiment of the seventh aspect by a the use of a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect for the detection of glucagon and/or GIP.

The problem underlying the present invention is solved in an eighth aspect which is also the first embodiment of the eighth aspect by a method for the screening of an antagonist of an activity mediated by glucagon and/or GIP comprising the following steps:
providing a candidate antagonist of the activity mediated by glucagon and/or GIP,
providing a nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect,
providing a test system which provides a signal in the presence of an antagonist of the activity mediated by glucagon and/or GIP, and
determining whether the candidate antagonist of the activity mediated by glucagon and/or GIP is an antagonist of the activity mediated by glucagon and/or GIP.

The problem underlying the present invention is solved in a ninth aspect which is also the first embodiment of the ninth aspect, by a kit for the detection of glucagon comprising a nucleic acid molecule according to any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect.

The problem underlying the present invention is solved in a tenth aspect which is also the first embodiment of the tenth aspect, by a method for the detection of a nucleic acid as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect in a sample, wherein the method comprises the steps of:
a) providing a capture probe, wherein the capture probe is at least partially complementary to a first part of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect, and a detection probe, wherein the detection probe is at least partially complementary to a second part of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th}and 59^{th} embodiment of the first aspect, or, alternatively, the capture probe is at least partially complementary to a second part of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{th}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect and the detection probe is at least partially complementary to the first part of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect;
b) adding the capture probe and the detection probe separately or combined to a sample containing the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{th}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{th}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect or presumed to contain the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect;
c) allowing the capture probe and the detection probe to react either simultaneously or in any order sequentially with the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect or part thereof;
d) optionally detecting whether or not the capture probe is hybridized to the nucleic acid molecule as defined any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th} 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect provided in step a); and
e) detecting the complex formed in step c) consisting of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{rd}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect and the capture probe and the detection probe.

In a second embodiment of the tenth aspect which is also an embodiment of the first embodiment of the tenth aspect, the detection probe comprises a detection means, and/or wherein the capture probe is immobilized to a support, preferably a solid support.

In a third embodiment of the tenth aspect which is also an embodiment of the first and the second embodiment of the tenth aspect, any detection probe which is not part of the complex formed in step c) is removed from the reaction so that in step e) only a detection probe which is part of the complex, is detected.

In a fourth embodiment of the tenth aspect which is also an embodiment of the first, second and third embodiment of the tenth aspect, step e) comprises the step of comparing the signal generated by the detection means when the capture probe and the detection probe are hybridized in the presence of the nucleic acid molecule as defined in any one of the first, second, third, fourth, fifth, sixth, seventh, eighth, ninth, tenth, eleventh, twelfth, 13^{th}, 14^{th}, 15^{th}, 16^{th}, 17^{th}, 18^{th}, 19^{th}, 20^{th}, 21^{st}, 22^{nd}, 23^{th}, 24^{th}, 25^{th}, 26^{th}, 27^{th}, 28^{th}, 29^{th}, 30^{th}, 31^{st}, 32^{nd}, 33^{rd}, 34^{th}, 35^{th}, 36^{th}, 37^{th}, 38^{th}, 39^{th}, 40^{th}, 41^{st}, 42^{nd}, 43^{rd}, 44^{th}, 45^{th}, 46^{th}, 47^{th}, 48^{th}, 49^{th}, 50^{th}, 51^{st}, 52^{nd}, 53^{rd}, 54^{th}, 55^{th}, 56^{th}, 57^{th}, 58^{th} and 59^{th} embodiment of the first aspect or part thereof, and in the absence of said nucleic acid molecule or part thereof.

While not wishing to be bound by any theory, the present inventors have found that the nucleic acid molecule according to the present invention binds specifically and with high affinity to glucagon, thereby inhibiting the binding of glucagon to its glucagon receptor and/or is thus, either directly or indirectly, useful in and used for the treatment of diabetes, diabetic complication, diabetic condition and/or hyperglucagonemia. Furthermore, the instant inventors have found that the nucleic acid molecule according to the present invention is suitable to block the interaction of glucagon with the glucagon receptor. Insofar, the nucleic acid molecule according to the present invention can also be viewed as an antagonist of the glucagon receptor and, respectively, as an antagonist of the effects of glucagon, in particular the effects of glucagon on its receptor.

An antagonist to glucagon is a molecule that binds to glucagon—such as the nucleic acid molecules according to the present invention—and inhibits the function of glucagon, preferably in an in vitro assay or in an in vivo model as described in the Examples.

As to the various diseases, conditions and disorders which may be treated or prevented by using the nucleic acid molecule according to the present invention or compositions, preferably pharmaceutical compositions comprising the same, it has to be acknowledged that such diseases, conditions and disorders are those which are described herein, including and in particular those described and set forth in the introductory part of the instant application. Insofar, the respective passages of the specification and the introductory part of the specification form an integral part of the present disclosure teaching the suitability of the nucleic acid molecule of the present invention for the prevention and treatment, respectively, for said diseases, conditions, and disorders.

Additionally, a nucleic molecule according to the present invention is preferred if the physiological effect of the glucagon—glucagon receptor axis is related to higher plasma levels of glucagon.

As used herein the term glucagon refers to any glucagon including, but not limited to, mammalian glucagon. Preferably, the mammalian glucagon is selected from the group comprising human, rat, mouse, monkey, pig, rabbit, hamster, dog, cheep, chicken and bovine glucagon (see glucagon species alignment in FIG. 22). More preferably the glucagon is human glucagon. The amino acid sequence of the various glucagons are known to the person skilled in the art and, among others, depicted in FIG. 22.

An antagonist to glucagon is a molecule that binds to glucagon—such as the nucleic acid molecule according to the present invention—and inhibts the function of glucagon, preferably in an in vitro assay or in an in vivo model as described in the Examples.

Moreover, the present inventors have found that nucleic acid molecule of Type B according to the present invention inhibits the binding of glucagon to its glucagon receptor and the binding of GIP to its receptor. Furthermore, the nucleic acid molecule of Type B according to the present invention is suitable to block the interaction of glucagon with the glucagon receptor and of GIP with the GIP receptor. Insofar, the nucleic acid molecule of Type B according to the present invention can also be viewed as an antagonist of the glucagon receptor and as antagonists of the GIP receptor.

An antagonist to GIP is a molecule that binds to GIP—such as the nucleic acid molecule according to the present invention—and inhibts the function of GIP, preferably in an in vitro assay or in an in vivo model as described in the Examples.

As used herein the term GIP refers to any GIP including, but not limited to, mammalian GIP. More preferably the GIP is human GIP. The amino acid sequence of GIP is known to the person skilled in the art and, among others, represented by SEQ ID NO: 168 disclosed herein.

It is within the present invention that the nucleic acid according to the present invention is a nucleic acid molecule. Insofar the terms nucleic acid and nucleic acid molecule are used herein in a synonymous manner if not indicated to the contrary. Moreover, such nucleic acid(s) is/are preferably also referred to herein as the nucleic acid molecule(s) according to the present invention, the nucleic acid(s) according to the present invention, the inventive nucleic acid(s) or the inventive nucleic acid molecule(s).

The features of the nucleic acid according to the present invention as described herein can be realised in any aspect of the present invention where the nucleic acid is used, either alone or in any combination.

As outlined in more detail herein, the present inventors have identified a number of different glucagon binding nucleic acid molecules, whereby the nucleic acid molecules can be characterised in terms of stretches of nucleotides which are also referred to herein as disclosed (see Example 1). As experimentally shown in example 8 the inventors could surprisingly demonstrate in several systems that nucleic acid molecules according to the present invention are suitable for the treatment of diabetes.

Each of the different types of glucagon binding nucleic acid molecules of the invention that bind to glucagon and/or GIP comprises three different stretches of nucleotides: a first terminal stretch of nucleotides, a central stretch of nucleotides and a second terminal stretch of nucleotides. In general, glucagon binding nucleic acid molecules of the present invention comprise at their 5′-end and the 3′-end each one of the terminal stretches of nucleotides, i.e. the first terminal stretch of nucleotides or the second terminal stretch of nucleotides (also referred to as 5′-terminal stretch of nucleotides and 3′-terminal stretch of nucleotides). The first terminal stretch of nucleotides and the second terminal stretch of nucleotides can, in principle due to their base complementarity, hybridize to each other, whereby upon hybridization a double-stranded structure is formed. However, such hybridization is not necessarily realized in the molecule under physiological and/or non-physiological conditions. The three stretches of nucleotides of glucagon binding nucleic acid molecules—the first terminal stretch of nucleotides, the central stretch of nucleotides and second terminal stretch of nucleotides—are arranged to each other in 5′→3′-direction: the first terminal stretch of nucleotides—the central stretch of nucleotides—the second terminal stretch of nucleotides. Alternatively, the second terminal stretch of nucleotides, the central stretch of nucleotides and the terminal first stretch of nucleotides are arranged to each other in 5′→3′-direction.

The differences in the sequences of the defined stretches between the different glucagon binding nucleic acid molecules may influence the binding affinity to glucagon and/or GIP. Based on binding analysis of the different glucagon binding nucleic acid molecules of the present invention the central stretch and the nucleotides forming the same are individually and more preferably in their entirety essential for binding to glucagon and/or GIP.

The terms ‘stretch’ and stretch of nucleotides' are used herein in a synonymous manner if not indicated to the contrary.

In a preferred embodiment the nucleic acid molecule according to the present invention is a single nucleic acid molecule. In a further embodiment, the single nucleic acid molecule is present as a multitude of the single nucleic acid molecule or as a multitude of the single nucleic acid molecule species.

It will be acknowledged by the ones skilled in the art that the nucleic acid molecule in accordance with the invention preferably consists of nucleotides which are covalently linked to each other, preferably through phosphodiester links or linkages.

It is within the present invention that the nucleic acid molecule according to the present invention comprises two or more stretches or part(s) thereof that can, in principle, hybridise with each other. Upon such hybridisation a double-stranded structure is formed. It will be acknowledged by the ones skilled in the art that such hybridisation may or may not occur, particularly under in vitro and/or in vivo conditions. Also, in case of hybridisation, such hybridisation does not necessarily occur over the entire length of the two stretches where, at least based on the rules for base pairing, such hybridisation and thus formation of a double-stranded structure may, in principle, occur. As preferably used herein, a double-stranded structure is a part of a nucleic acid molecule or a structure formed by two or more separate strands or two spatially separated stretches of a single strand of a nucleic acid molecule, whereby at least one, preferably two or more base pairs exist which are base pairing preferably in accordance with the Watson-Crick base pairing rules. It will also be acknowledged by the one skilled in the art that other base pairing such as Hoogsten base pairing may exist in or may form such double-stranded structure. It is also to be acknowledged that the feature that two stretches hybridize preferably indicates that such hybridization is assumed to happen due to base complementarity of the two stretches regardless of whether such hybridization actually occurs in vivo and/or in vitro.

In a preferred embodiment the term arrangement as used herein, means the order or sequence of structural or functional features or elements described herein in connection with the nucleic acids molecule(s) disclosed herein.

It will be acknowledged by the person skilled in the art that the nucleic acid molecule according to the present invention is capable of binding to glucagon and/or GIP. Without wishing to be bound by any theory, the present inventors assume that the glucagon binding and/or GIP binding results from a combination of three-dimensional structural traits or elements of the nucleic acid molecule of the present invention, which are caused by orientation and folding patterns of the primary sequence of nucleotides of the nucleic acid molecule of the invention forming such traits or elements, whereby preferably such traits or elements are the first terminal stretch of nucleotides, the central stretch of nucleotides and/or the second terminal stretch of nucleotides of the nucleic acid molecule of the present invention. It is evident that the individual trait or element may be formed by various different individual sequences the degree of variation of which may vary depending on the three-dimensional structure such element or trait has to form for mediating the binding of the nucleic acid molecule of the invention to glucagon and/or GIP. The overall binding characteristic of the nucleic acid of the present invention results from the interplay of the various elements and traits, respectively, which ultimately results in the interaction of the nucleic acid molecule of the present invention with its target, i. e. glucagon and GIP, respectively. Again without wishing to be bound by any theory, the central stretch of nucleotides that is characteristic for nucleic acid of the present invention is important for mediating the binding of the nucleic acid molecule of the invention with glucagon and/or GIP. Accordingly, the nucleic acid molecule according to the present invention is capable of interacting with glucagon. Also, it will be acknowledged by the person skilled in the art that the nucleic acid molecule according to the present invention is an antagonist to glucagon and/or GIP. Because of this the nucleic acid molecule according to the present invention is suitable for the treatment and prevention, respectively, of any disease or condition which is associated with or caused by glucagon and/or GIP. Such diseases and conditions may be taken from the prior art which establishes that glucagon and/or GIP is involved or associated with said diseases and conditions, respectively, and which is incorporated herein by reference providing the scientific rationale for the therapeutic use of the nucleic acid molecule of the present invention.

The nucleic acid molecule according to the present invention shall also comprise a nucleic acid molecule which is essentially homologous to the particular nucleotide sequences disclosed herein. The term substantially homologous shall be understood such as the homology is at least 75%, preferably at least 85%, more preferably at least 90% and most preferably more that at least 95%, 96%, 97%, 98% or 99%.

The actual percentage of homologous nucleotides present in a nucleic acid molecule according to the present invention will depend on the total number of nucleotides present in the nucleic acid. The percent modification can be calculated based upon the total number of nucleotides present in the nucleic acid molecule.

The homology between two nucleic acid molecules can be determined as known to the person skilled in the art. More specifically, a sequence comparison algorithm may be used for calculating the percent sequence homology for a test sequence(s) relative to a reference sequence, based on the designated program parameters. The test sequence is preferably the sequence or nucleic acid molecule which is said to be homologous or to be tested whether it is homologous, and if so, to what extent, to a different nucleic acid molecule, whereby such different nucleic acid molecule is also referred to as the reference sequence. In an embodiment, the reference sequence is a nucleic acid molecule as described herein, preferably a nucleic acid molecule having a sequence according to any one of SEQ ID NO: 6, SEQ ID NO: 7, SEQ ID NO: 43, SEQ ID NO: 47, SEQ ID NO: 48, SEQ ID NO: 91, SEQ ID NO: 92, SEQ ID NO: 71, SEQ ID NO: 81, SEQ ID NO: 82, SEQ ID NO: 88, SEQ ID NO: 89, SEQ ID NO: 90, SEQ ID NO: 50, SEQ ID NO: 54 or SEQ ID NO: 59. Optimal alignment of sequences for comparison can be conducted, e.g., by the local homology algorithm of Smith & Waterman (Smith & Waterman, 1981), by the homology alignment algorithm of Needleman & Wunsch (Needleman & Wunsch, 1970), by the search for similarity method of Pearson & Lipman (Pearson & Lipman, 1988), by computerized implementations of these algorithms (GAP, BESTFIT, FASTA, and TFASTA in the Wisconsin Genetics Software Package, Genetics Computer Group, 575 Science Dr., Madison, Wis.), or by visual inspection.

One example of an algorithm that is suitable for determining percent sequence identity is the algorithm used in the basic local alignment search tool (hereinafter “BLAST”), see, e.g. Altschul et al (Altschul et al. 1990 and Altschul et al, 1997). Software for performing BLAST analyses is publicly available through the National Center for Biotechnology Information (hereinafter “NCBI”). The default parameters used in determining sequence identity using the software available from NCBI, e.g., BLASTN (for nucleotide sequences) and BLASTP (for amino acid sequences) are described in McGinnis et al (McGinnis et al, 2004).

The nucleic acid molecule according to the present invention shall also comprise a nucleic acid molecule which has a certain degree of identity relative to the nucleic acid(s) of the present invention disclosed herein and defined by it/their nucleotide sequence. More preferably, the instant invention also comprises those nucleic acid molecules which have an identity of at least 75%, preferably at least 85%, more preferably at least 90% and most preferably more than at least 95%, 96%, 97%, 98% or 99% relative to the nucleic acid molecule of the present invention defined by their nucleotide sequence or a part thereof.

The term inventive nucleic acid or nucleic acid molecule according to the present invention shall also comprise a nucleic acid molecule comprising a nucleic acid sequence disclosed herein or part thereof, such as, e.g., a metabolite or derivative of the nucleic acid according to the present invention, preferably to the extent that the nucleic acid molecule or said parts are involved in the or capable of binding to glucagon. Such a nucleic acid molecule may be derived from the ones disclosed herein by, e.g., truncation. Truncation may be related to either one or both of the ends of a nucleic acid molecule of the present invention as disclosed herein. Also, truncation may be related to the inner sequence of nucleotides, i.e. it may be related to one or several of the nucleotide(s) between the 5′ terminal nucleotide and the 3′ terminal nucleotide, respectively. Moreover, truncation shall comprise the deletion of as little as a single nucleotide from the sequence of a nucleic acid molecule of the present invention disclosed herein. Truncation may also be related to more than one stretch of nucleotides of the nucleic acid molecule of the present invention, whereby the stretch of nucleotides can be as little as one nucleotide long. The binding of a nucleic acid molecule according to the present invention can be determined by the ones skilled in the art using routine experiments or by using or adopting a method as described herein, preferably as described herein in the example part.

The nucleic acid molecule according to the present invention may be either a D-nucleic acid molecule or an L-nucleic acid molecule. Preferably, the nucleic acid molecule according to the present invention is an L-nucleic acid molecule.

It is also within the present invention that, in an embodiment, each and any of the nucleic acid molecules described herein in their entirety in terms of their nucleic acid sequence(s) are limited to the particular indicated nucleotide sequence(s). In other words, the terms “comprising” or “comprise(s)” shall be interpreted in such embodiment in the meaning of containing or consisting of.

It is also within the present invention that the nucleic acid molecule according to the present invention is part of a longer nucleic acid whereby this longer nucleic acid comprises several parts whereby at least one such part is a nucleic acid molecule of the present invention, or a part thereof. The other part(s) of such longer nucleic acid can be either one or several D-nucleic acid(s) or L-nucleic acid(s). Any combination may be used in connection with the present invention. These other part(s) of the longer nucleic acid can exhibit a function which is different from binding, preferably from binding to glucagon and/or GIP. One possible function is to allow interaction with other molecules, whereby such other molecules preferably are different from glucagon such as, e.g., for immobilization, cross-linking, detection or amplification. In a further embodiment of the present invention the nucleic acid molecule according to the invention comprises, as individual or combined moieties, several of the nucleic acid molecules of the present invention. Such nucleic acid comprising several of the nucleic acid molecules of the present invention is also encompassed by the term longer nucleic acid.

An L-nucleic acid as used herein is a nucleic acid or nucleic acid molecule consisting of L-nucleotides, preferably consisting completely of L-nucleotides.

A D-nucleic acid as used herein is nucleic acid or nucleic acid molecule consisting of D-nucleotides, preferably consisting completely of D-nucleotides.

The terms nucleic acid and nucleic acid molecule are used herein in an interchangeable manner if not explicitly indicated to the contrary.

Also, if not indicated to the contrary, any nucleotide sequence is set forth herein in 5′→3′ direction.

As preferably used herein any position of a nucleotide is determined or referred to relative to the 5′ end of a sequence, a stretch or a substretch containing such nucleotide. Accordingly, a second nucleotide is the second nucleotide counted from the 5′ end of the sequence, stretch and substretch, respectively. Also, in accordance therewith, a penultimate nucleotide is the second nucleotide counted from the 3′ end of a sequence, stretch and substretch, respectively.

Irrespective of whether the nucleic acid molecule of the invention consists of D-nucleotides, L-nucleotides or a combination of both with the combination being e.g. a random combination or a defined sequence of stretches consisting of at least one L-nucleotide and at least one D-nucleic acid, the nucleic acid may consist of desoxyribonucleotide(s), ribonucleotide(s) or combinations thereof.

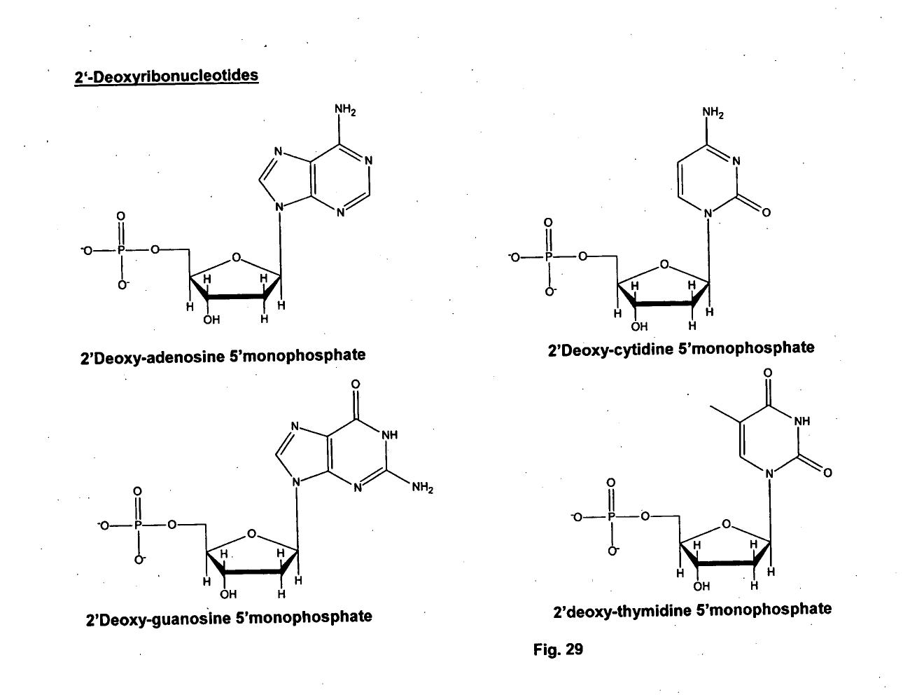

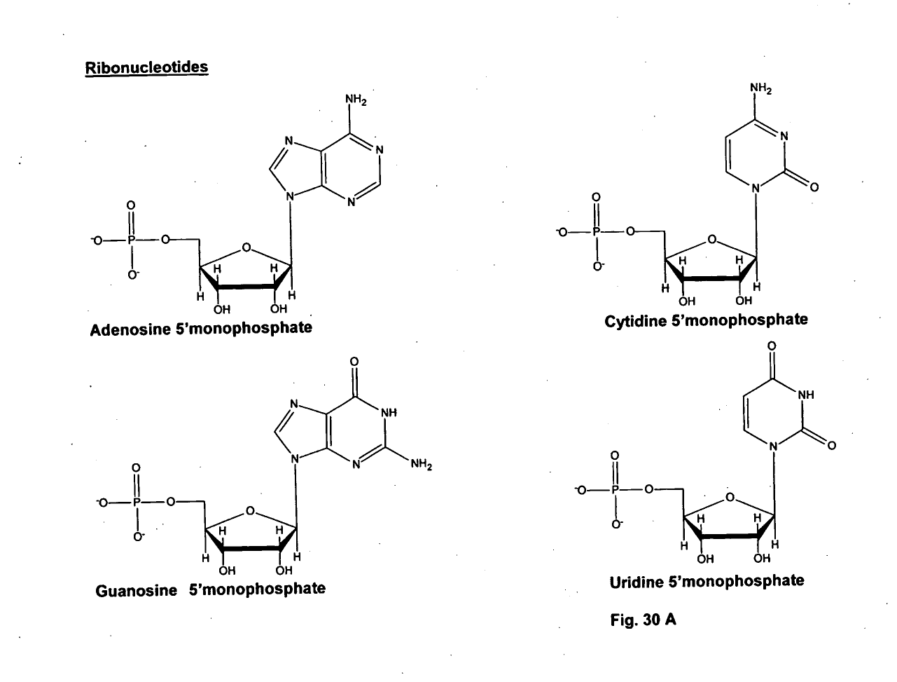

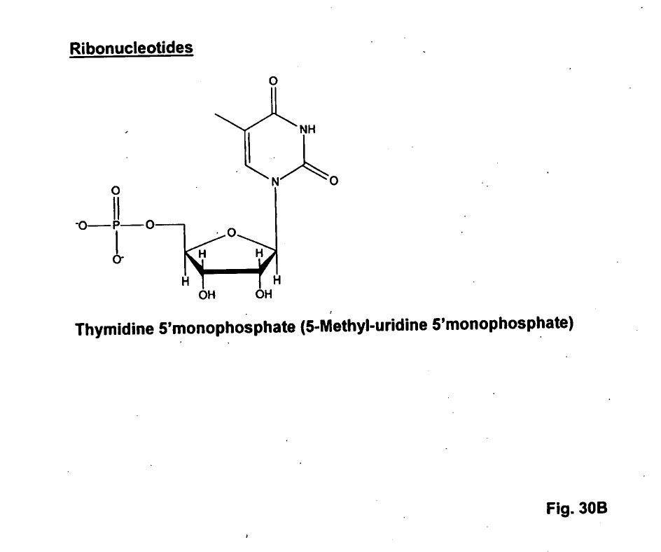

It is also within the present invention that the nucleic acid molecule consists of both ribonucleotides and 2′deoxyribonucleotides. The 2′deoxyribonucleotides and ribonucleotides are shown in FIGS. 29 and 30A-B. In order to distinguish between ribonucleotides and 2′deoxyribonucleotides in the sequences of the nucleic acid molecules according to the present invention the following reference code is used herein.

The nucleic acid molecule according to the present invention mainly consists of 2′deoxyribonucleotides, wherein preferably
G is 2′deoxy-guanosine-5′-monophosphate,
C is 2′deoxy-cytidine-5′-monophosphate,
A is 2′deoxy-adenosine-5′-monophosphate,
T is 2′deoxy-thymidine-5′-monophosphate,
rG is guanosine-5′-monophosphate,
rC is cytidine 5′-monophosphate,
rA is adenosine-5′-monophosphate,
rU is uridine-5′-monophosphate,
rT is thymidine-5′-monophosphate-.

The nucleic acid molecule according to the present invention mainly consists of ribonucleotides, wherein preferably
G is guanosine-5′-monophosphate,
C is cytidine 5′-monophosphate,
A is adenosine-5′-monophosphate,
U is uridine-5′monophosphate,
dG is 2′deoxy-guanosine-5′-monophosphate,
dC is 2′deoxy-cytidine-5′monophosphate,
dA is 2′deoxy-adenosine-5′-monophosphate,
dT is 2′deoxy-thymidine-5′-monophosphate.

Designing the nucleic acid molecule of the invention as an L-nucleic acid molecule is advantageous for several reasons. L-nucleic acid molecules are enantiomers of naturally occurring nucleic acids. D-nucleic acid molecules, however, are not very stable in aqueous solutions and particularly in biological systems or biological samples due to the widespread presence of nucleases. Naturally occurring nucleases, particularly nucleases from animal cells are not capable of degrading L-nucleic acids. Because of this, the biological half-life of an L-nucleic acid molecule is significantly increased in such a system, including the animal and human body. Due to the lacking degradability of L-nucleic acid molecules no nuclease degradation products are generated and thus no side effects arising therefrom observed in such a system including the animal and human body. This aspect distinguishes L-nucleic acid molecules from factually all other compounds which are used in the therapy of diseases and/or disorders involving the presence of glucagon. An L-nucleic acid molecule which specifically binds to a target molecule through a mechanism different from Watson Crick base pairing, or an aptamer which consists partially or completely of L-nucleotides, particularly with those parts of the aptamer being involved in the binding of the aptamer to the target molecule, is also called a spiegelmer. Aptamers and spiegelmers as such are known to a person skilled in the art and are, among others, described in ‘The Aptamer Handbook’ (eds. Klussmann, 2006).

It is also within the present invention that the nucleic acid molecule of the invention, regardless whether it is are present as a D-nucleic acid, L-nucleic acid or D,L-nucleic acid or whether it is DNA or RNA, may be present as single stranded or double stranded nucleic acid molecule. Typically, the nucleic acid molecule is a single stranded nucleic acid molecule which exhibits a defined secondary structure due to its primary sequence and may thus also form a tertiary structure. The nucleic acid molecule, however, may also be double stranded in the meaning that two strands which are complementary or partially complementary to each other are hybridised to each other.

The nucleic acid molecule of the invention may be modified. Such modification may be related to the single nucleotide of the nucleic acid molecule and is well known in the art. Examples for such modification are described by, among others, Venkatesan et al. (Venkatesan, Kim et al. 2003) and Kusser (Kusser 2000). Such modification can be a H atom, a F atom or O—CH₃ group or NH₂-group at the 2′ position of one, several of all of the individual nucleotides of which the nucleic acid molecule consists. Also, the nucleic acid molecule according to the present invention can comprise at least one LNA nucleotide. In an embodiment the nucleic acid molecule according to the present invention consists of LNA nucleotides.

In an embodiment, the nucleic acid molecule according to the present invention may be a multipartite nucleic acid molecule. A multipartite nucleic acid molecule as used herein is a nucleic acid molecule which consists of at least two separate nucleic acid strands. These at least two nucleic acid strands form a functional unit whereby the functional unit is a ligand to a target molecule and, preferably an antagonist to the target molecule, in the instant case of glucagon and/or GIP. The at least two nucleic acid strands may be derived from any of the nucleic acid molecule of the invention by either cleaving a nucleic acid molecule of the invention to generate at least two strands or by synthesising one nucleic acid molecule corresponding to a first part of the full-length nucleic acid molecule of the invention and another nucleic acid molecule corresponding to another part of the full-length nucleic acid molecule of the invention. Depending on the number of parts forming the full-length nucleic acid molecules the corresponding number of parts having the required nucleotide sequence will be synthesized It is to be acknowledged that both the cleavage approach and the synthesis approach may be applied to generate a multipartite nucleic acid molecule where there are more than two strands as exemplified above. In other words, the at least two separate nucleic acid strands are typically different from two strands being complementary and hybridising to each other although a certain extent of complementarity between said at least two separate nucleic acid strands may exist and whereby such complementarity may result in the hybridisation of said separate strands.

Finally, it is also within the present invention that a fully closed, i.e. circular structure for the nucleic acid molecule according to the present invention is realized, i.e. that the nucleic acid molecule according to the present invention are closed in an embodiment, preferably through a covalent linkage, whereby more preferably such covalent linkage is made between the 5′ end and the 3′ end of the nucleic acid sequence of the nucleic acid molecule of the invention as disclosed herein or any derivative thereof.

A possibility to determine the binding constants of the nucleic acid molecule according to the present invention is the use of the methods as described in examples 3 and 4 which confirms the above finding that the nucleic acids according to the present invention exhibit a favourable K_{D} value range. An appropriate measure in order to express the intensity of the binding between the individual nucleic acid molecule and the target which is in the present case glucagon is the so-called K_{D} value which as such as well as the method for its determination are known to the one skilled in the art.

Preferably, the K_{D} value shown by the nucleic, acid according to the present invention is below 1 μM. A K_{D} value of about 1 μM is said to be characteristic for a non-specific binding of a nucleic acid to a target. As will be acknowledged by the ones skilled in the art, the K_{D} value of a group of compounds such as various embodiment of the nucleic acid molecule according to the present invention is within a certain range. The above-mentioned K_{D} of about 1 μM is a preferred upper limit for the K_{D} value. The lower limit for the K_{D} of target binding nucleic acids such as the one of the nucleic acid molecule of the invention can be as little as about 10 picomolar or can be higher. It is within the present invention that the K_{D} values of individual nucleic acids binding to glucagon is preferably within this range. Preferred ranges of K_{D} values can be defined by choosing any first number within this range and any second number within this range. Preferred upper K_{D} values are 250 nM and 100 nM, preferred lower K_{D} values are 50 nM, 10 nM, 1 nM, 100 pM and 10 pM. The more preferred upper K_{D} value is 10 nM, the more preferred lower K_{D} value is 100 pM.

In addition to the binding properties of the nucleic acid molecule according to the present invention, the nucleic acid molecule according to the present invention inhibits the function of the respective target molecule which is in the present case glucagon and/or GIP. The inhibition of the function of glucagon and/or GIP—for instance the stimulation of the respective receptors as described previously—is achieved by the binding of a nucleic acid molecule according to the present invention to glucagon and/or GIP and forming a complex of the nucleic acid molecule according to the present invention and glucagon and/or GIP. Such complex of a nucleic acid molecule of the present invention and glucagon and/or GIP cannot stimulate the receptors that normally are stimulated by glucagon and/or GIP, i.e. glucagon and/or GIP which is not present in a complex with a nucleic acid molecule of the invention. Accordingly, the inhibition of receptor function by a nucleic acid molecule according to the present invention is independent from the respective receptor that can be stimulated by glucagon and/or GIP, rather such inhibition results from preventing the stimulation of the receptor by glucagon and/or GIP by the nucleic acid molecule according to the present invention.

A possibility to determine the inhibitory constant of a nucleic acid molecule according to the present invention is the use of the methods as described in example 5 which confirms the above finding that the nucleic acid according to the present invention exhibits a favourable inhibitory constant which allows the use of said nucleic acid molecule in a therapeutic treatment scheme. An appropriate measure for expressing the intensity of the inhibitory effect of the individual nucleic acid molecule on the interaction of the target which is in the present case glucagon, and the respective receptor, is the so-called half maximal inhibitory concentration (abbr. IC₅₀) which as such as well as the method for its determination are known to the one skilled in the art.

Preferably, the IC₅₀ value shown by the nucleic acid molecule according to the present invention is below 1 μM. An IC₅₀ value of about 1 μM is said to be characteristic for a non-specific inhibition of target functions, preferably the inhibition of the activation of the target receptor by the target, by a nucleic acid molecule. As will be acknowledged by the ones skilled in the art, the IC₅₀ value of a group of compounds such as various embodiments of the nucleic acid molecule according to the present invention is within a certain range. The above-mentioned IC₅₀ of about 1 μM is a preferred upper limit for the IC₅₀ value. The lower limit for the IC₅₀ of a target binding nucleic acid molecule of the invention can be as little as about 10 picomolar or can be higher. It is within the present invention that the IC₅₀ values of individual nucleic acids of the invention binding to glucagon is preferably within this range. Preferred ranges can be defined by choosing any first number within this range and any second number within this range. Preferred upper IC₅₀ values are 250 nM and 100 nM, preferred lower IC₅₀ values are 50 nM, 10 nM, 1 nM, 100 pM and 10 pM. The more preferred upper IC₅₀ value is 5 nM, the more preferred lower IC₅₀ value is 1 nM.

The nucleic acid molecule according to the present invention may have any length provided that it is still capable of binding to the target molecule which is in the instant case glucagon and/or GIP. It will be acknowledged in the art that there are preferred lengths of the nucleic acid molecule according to the present inventions. Typically, the length is between 15 and 120 nucleotides. It will be acknowledged by the ones skilled in the art that any integer between 15 and 120 is a possible length for a nucleic acid molecule according to the present invention. More preferred ranges for the length of a nucleic acid molecule according to the present invention are lengths of about 20 to 100 nucleotides, about 20 to 80 nucleotides, about 20 to 60 nucleotides, about 20 to 54 nucleotides and about 39 to 44 nucleotides.

It is within the present invention that the nucleic acid molecule of the present invention comprises a moiety which preferably is a high molecular weight moiety and/or which preferably allows to modify the characteristics of the nucleic acid molecule in terms of, among others, residence time in the animal body, preferably the human body. A particularly preferred embodiment of such modification is PEGylation and HESylation of the nucleic acids according to the present invention. As used herein PEG stands for poly(ethylene glycole) and HES for hydroxyethyl starch. PEGylation as preferably used herein is the modification of a nucleic acid molecule according to the present invention whereby such modification consists of a PEG moiety which is attached to a nucleic acid molecule according to the present invention. HESylation as preferably used herein is the modification of a nucleic acid molecule according to the present invention whereby such modification consists of a HES moiety which is attached to a nucleic acid molecule according to the present invention. These modifications as well as the process of modifying a nucleic acid molecule using such modifications, is described in European patent application EP 1 306 382, the disclosure of which is herewith incorporated in its entirety by reference.

In the case of PEG being such high molecular weight moiety the molecular weight is preferably about 20,000 to about 120,000 Da, more preferably from about 30,000 to about 80,000 Da and most preferably about 40,000 Da. In the case of HES being such high molecular weight moiety the molecular weight is preferably from about 50 kDa to about 1000 kDa, more preferably from about 100 kDa to about 700 kDa and most preferably from 200 kDa to 500 kDa. HES exhibits a molar substitution of 0.1 to 1.5, more preferably of 1 to 1.5 and exhibits a substitution grade expressed as the C2/C6 ratio of approximately 0.1 to 15, preferably of approximately 3 to 10. The process of HES modification is, e.g., described in German patent application DE 1 2004 006 249.8 the disclosure of which is herewith incorporated in its entirety by reference.

The modification can, in principle, be made to the nucleic acid molecule of the present invention at any position thereof. Preferably such modification is made either to the 5′-terminal nucleotide, the 3′-terminal nucleotide and/or any nucleotide between the 5′ nucleotide and the 3′ nucleotide of the nucleic acid molecule.

The modification and preferably the PEG and/or HES moiety can be attached to the nucleic acid molecule of the present invention either directly or indirectly, preferably indirectly through a linker. It is also within the present invention that the nucleic acid molecule according to the present invention comprises one or more modifications, preferably one or more PEG and/or HES moiety. In an embodiment the individual linker molecule attaches more than one PEG moiety or HES moiety to a nucleic acid molecule according to the present invention. The linker used in connection with the present invention can itself be either linear or branched. This kind of linkers are known to the ones skilled in the art and are further described in international patent applications WO2005/074993 and WO2003/035665.

In a preferred embodiment the linker is a biodegradable linker. The biodegradable linker allows to modify the characteristics of the nucleic acid molecule according to the present invention in terms of, among other, residence time in an animal body, preferably in a human body, due to release of the modification from the nucleic acid molecule according to the present invention. Usage of a biodegradable linker may allow a better control of the residence time of the nucleic acid molecule according to the present invention. A preferred embodiment of such biodegradable linker is a biodegradable linker as described in, but not limited to, international patent applications WO2006/052790, WO2008/034122, WO2004/092191 and WO2005/099768.

It is within the present invention that the modification or modification group is a biodegradable modification, whereby the biodegradable modification can be attached to the nucleic acid molecule of the present invention either directly or indirectly, preferably through a linker. The biodegradable modification allows modifying the characteristics of the nucleic acid molecule according to the present invention in terms of, among other, residence time in an animal body, preferably in a human body, due to release or degradation of the modification from the nucleic acid molecule according to the present invention. Usage of a biodegradable modification may allow a better control of the residence time of the nucleic acid molecule according to the present invention. A preferred embodiment of such biodegradable modification is biodegradable as described in, but not restricted to, international patent applications WO2002/065963, WO2003/070823, WO2004/113394 and WO2000/41647, preferably in WO2000/41647, page 18, line 4 to 24.

Beside the modifications as described above, other modifications can be used to modify the characteristics of the nucleic acid molecule according to the present invention, whereby such other modifications may be selected from the group of proteins, lipids such as cholesterol and sugar chains such as amylase, dextran etc.

Without wishing to be bound by any theory, by modifying the nucleic acid molecule according to the present invention with a high molecular weight moiety such as a polymer and more particularly one or several of the polymers disclosed herein, which are preferably physiologically acceptable, the excretion kinetic of the thus modified nucleic acid molecule of the invention is changed. More particularly, due to the increased molecular weight of the tus modified nucleic acid molecule of the invention and due to the nucleic acid molecule of the invention not being subject to metabolism particularly when in the L form, i.e. being an L-nucleic acid molecule, excretion from an animal body, preferably from a mammalian body and more preferably from a human body is decreased. As excretion typically occurs via the kidneys, the present inventors assume that the glomerular filtration rate of the thus modified nucleic acid molecule is significantly reduced compared to a nucleic acid molecule not having this kind of high molecular weight modification which results in an increase in the residence time of the modified nucleic acid molecule in the animal body. In connection therewith it is particularly noteworthy that, despite such high molecular weight modification the specificity of the nucleic acid molecule according to the present invention is not affected in a detrimental manner. Insofar, the nucleic acid molecule according to the present invention has among others, the surprising characteristic—which normally cannot be expected from a pharmaceutically active compound—that a pharmaceutical formulation providing for a sustained release is not necessarily required for providing a sustained release of the nucleic acid molecule according to the present invention. Rather, the nucleic-acid molecule according to the present invention in its modified form comprising a high molecular weight moiety, can as such already be used as a sustained release-formulation as it acts, due to its modification, already as if it was released from a sustained-release formulation. Insofar, the modification(s) of the nucleic acid molecule according to the present invention as disclosed herein and the thus modified nucleic acid molecule according to the present invention and any composition comprising the same may provide for a distinct, preferably controlled pharmacokinetics and biodistribution thereof. This also includes residence time in the circulation of the animal and human body and distribution to tissues in such animal and human. Such modifications are further described in the patent application WO2003/035665.

However, it is also within the present invention that the nucleic acid molecule according to the present invention does not comprise any modification and particularly no high molecular weight modification such as PEG or HES. Such embodiment is particularly preferred when the nucleic acid molecule according to the present invention shows preferential distribution to any target organ or tissue in the body or when a fast clearance of the nucleic acid molecule according to the present invention from the body after administration is desired. A nucleic acid molecule according to the present invention as disclosed herein with a preferential distribution profile to any target organ or tissue in the body would allow establishment of effective local concentrations in the target tissue while keeping systemic concentration of the nucleic acid molecule low. This would allow the use of low doses which is not only beneficial from an economic point of view, but also reduces unnecessary exposure of other tissues to the nucleic acid molecule, thus reducing the potential risk of side effects. Fast clearance of the nucleic acid molecule according to the present invention from the body after administration might be desired, among others, in case of in vivo imaging or specific therapeutic dosing requirements using the nucleic acid molecule according to the present invention or medicaments comprising the same.

The nucleic acid molecule according to the present invention, and/or the antagonist according to the present invention may be used for the generation or manufacture of a medicament. Such medicament or a pharmaceutical composition according to the present invention contains at least one species of a nucleic acid molecule of the invention capable of binding to glucagon and/or GIP optionally together with further pharmaceutically active compounds, whereby the nucleic acid molecule of the invention preferably acts as pharmaceutically active compound itself. Such medicaments comprise in preferred embodiments at least a pharmaceutically acceptable carrier. Such carrier may be, e.g., water, buffer, PBS, glucose solution, preferably a 5% glucose, salt balanced solution, citrate, starch, sugar, gelatine or any other acceptable carrier substance. Such carriers are generally known to the one skilled in the art. It will be acknowledged by the person skilled in the art that any embodiments, use and aspects of or related to the medicament of the present invention is also applicable to the pharmaceutical composition of the present invention and vice versa.

The indication, diseases and disorders for the treatment and/or prevention of which the nucleic acid molecule, the pharmaceutical compositions and medicaments in accordance with or prepared in accordance with the present invention result from the involvement, either direct or indirect, of glucagon in the respective pathogenetic mechanism.

Based on the involvement of glucagon in pathways relevant for or involved in diabetes, it is evident that the nucleic acid molecule of the present invention, the pharmaceutical compositions containing one or several species of the nucleic acid molecule of the present invention and the medicaments containing one or several thereof can be used in the treatment and/or prevention of said disease, disorders and diseased conditions. Accordingly, such diseases and/or disorders and/or diseased conditions include, but are not limited to, type 1 diabetes, type 2 diabetes (including gestational diabetes), diabetic complications, diabetic conditions and/or sequelae of diabetes mellitus and hyperglucagonemia and Alström-Syndrome due to other causes, whereby the resulting complications are selected from the group comprising atherosclerosis, coronary artery disease, diabetic foot disease, diabetic retinopathy, proliferative diabetic retinopathy, diabetic macular edema, diabetic vitreoretinopathy, proliferative diabetic vitreoretinopathy, diabetic nephropathy, diabetic neuropathy, gestational diabetes mellitus, glucose intolerance, heart disease, high blood pressure, high cholesterol, impaired glucose tolerance, impotence, insulin resistance, kidney failure, metabolic syndrome, non-alcoholic fatty liver disease, non-alcoholic steatohepatitis with or without fibrosis, peripheral vascular disease, reduced glucose sensitivity, reduced insulin sensitivity, obesity, hepatic steatosis, hyperglycemia, diabetic ketoacidosis, and hyperosmolar hyperglycemic non-ketoic coma, weight loss necrolytic migratory erythema (NME), anemia, venous thrombosis in the present of normal coagulation function, neuropsychiatric manifestations (depression, dementia, insomnia, ataxia).

Of course, because the glucagon binding nucleic acid molecule according to the present inventions interact with or binds to glucagon and/or GIP, a skilled person will generally understand that the glucagon binding nucleic acid molecule according to the present invention can easily be used for the treatment, prevention and/or diagnosis of any disease as described herein of humans and animals. In connection therewith, it is to be acknowledged that the nucleic acid molecule according to the present invention can be used for the treatment and prevention of any of the diseases, disorder or condition described herein, irrespective of the mode of action underlying such disease, disorder and condition.

In the following the rational for the use of the nucleic acid molecule according to the present invention in connection with the various diseases, disorders and conditions is provided, thus rendering the claimed therapeutic, preventive and diagnostic applicability of the nucleic acid molecule according to the present invention plausible. In order to avoid any unnecessary repetition, it should be acknowledged that due to the involvement of the glucagon-glucagon receptor axis and/or the GIP-GIP receptor axis as outlined in connection therewith said axis may be addressed by the nucleic acid molecule according to the present invention such that the claimed therapeutic, preventive and diagnostic effect is achieved. It should furthermore be acknowledged that the particularities of the diseases, disorders and conditions, of the patients and any detail of the treatment regimen described in connection therewith, may be subject to preferred embodiments of the instant application.

In the majority of diabetic patients a paradoxical increase of circulating glucagon levels following a mixed meal or carbohydrate ingestion has been reported (Ohneda, Watanabe et al. 1978). This is viewed as a major contributor to increased postprandial blood glucose levels which play an important role in the pathophysiology of micro- and macrovascular complications in DM (Gin and Rigalleau 2000).

A wealth of peptidyl and non-peptidyl small-molecule glucagon receptor antagonists have been reported (Jiang and Zhang 2003). Some of these small-molecule antagonists, that generally have rather low affinities for the glucagon receptor, have been shown to lower fasting blood glucose or to block exogenous glucagon-stimulated elevation of blood glucose in animal models. A non-peptidyl small molecule glucagon receptor antagonist was shown to block glucagon-induced elevation of hepatic glucose production and blood glucose in humans in a dose-dependent fashion (Petersen and Sullivan 2001). More recently, the reduction of the glucagon receptor expression in db/db-mice by antisense oligonucleotides led to reductions of blood glucose, free fatty acids and triglycerides without development of hypoglycaemia (Liang, Osborne et al. 2004). These effects would be ideal for patients with DM2.

Beyond that, glucagon receptor knock-out mice were found to be viable and to show signs of only mild hypoglycemia, improved glucose tolerance and elevated glucagon levels. They are also resistant to diet-induced obesity (Conarello, Jiang et al. 2007), and have a higher insulin sensitivity which may be beneficial in 1-cell sparing (Sorensen, Winzell et al. 2006). Moreover, glucagon receptor knock-out mice were resistant to streptozotocin-induced “type 1 diabetes phenotype”, i.e. they showed normoglycemia in the fasted state and after oral and intraperitoneal glucose tolerance tests (Lee, Wang et al. 2011).

Neutralization of glucagon itself by monoclonal antibodies also led to an acute and sustained reduction of blood glucose, triglycerides, HbA1c, and hepatic glucose output (Brand, Rolin et al. 1994; Sorensen, Brand et al. 2006). However, because of their potential immunogenicity, these and other antibodies might not be a viable option for the long-term treatment of DM.

Essentially, attempts for therapeutic intervention through lowering glucagon levels/activity have yielded a lot of results supporting the concept of glucagon antagonism. However, such attempts have either lead to compounds not having enough potency or to compounds with inacceptable hepatic toxicity.

Type 1 diabetes mellitus (DM1) is characterized by an insulin deficiency which is in contrast to DM2 not a functional deficiency due to insulin resistance but an absolute deficiency due to pancreatic β-cell loss. DM1 is often referred to as juvenile diabetes as it mostly develops in children and young adults. In a recently published study glucagon receptor knock-out mice were resistant to streptozotocin-induced “type 1 diabetes phenotype”, i.e. they showed normoglycemia in the fasted state and after oral and intraperitoneal glucose tolerance tests (Lee, Wang et al. 2011).

In DM1 patients lack of insulin-dependent postprandial suppression of glucagon impairs glucose tolerance. An acute life-threatening complication of DM and a direct consequence of the glucagon-insulin-imbalance is diabetic ketoacidosis (abbr. DKA) subsequent to an excessive ketone body production and diabetic complications like hyperosmolar hyperglycemic non-ketoic coma (abbr. HHNK). In HHNK the osmotic effects of glycosuria result in impaired renal NaCl and thus water reabsorption leading to hypernatremia (Wahid, Naveed et al. 2007). DKA and HHNK can also be observed in insulin-dependent cases of DM2.

Neuroendocrine tumors are rare tumors that may lead to overexpression of the respective hormone that is usually produced by the cells they originate from. Thus hyperglucagonemia is caused by hyperplasia or neoplasia of glucagon-producing cells (glucagonoma), e.g. α-cell-derived neoplasms. Likewise a neoplasia of intestinal Langerhans cells, in which glicentin, oxyntomodulin and GLP-1 is produced from the glucagon gene transcript, may lead to the overexpression of these peptides or to the overexpression of glucagon if processing is skewed.

Hyperglucagonemia can lead to complications, such as diabetes mellitus, ketoacidosis and weight loss necrolytic migratory erythema (abbr. NME), anemia, venous thrombosis in the presence of normal coagulation function, neuropsychiatric manifestations (depression, dementia, insomnia, ataxia) and other symptoms (Griffing, Odeke et al. 2011),

GIP does not only induce insulin release as its name suggests, but may also play a role in lipid homeostasis and may be necessary for the development of obesity as shown by several animal studies (Asmar 2011): Daily administration of the GIP receptor antagonist Pro3-GIP for 50 days produced reduced body weight, decreased accumulation of adipose tissue, and marked improvements in levels of glucose, glycated hemoglobin and pancreatic insulin in older high fat fed diabetic mice, together with reduced triglyceride levels in muscle and liver. No change of high-fat diet intake was noted (McClean, Irwin et al. 2007). Pointing in the same direction, GIP receptor knock-out mice were found to be resistant to the development of obesity while wild-type mice fed the same high-fat diet exhibited both hypersecretion of GIP and extreme visceral and subcutaneous fat deposition with insulin resistance (Miyawaki, Yamada et al. 2002). However, the early insulin response after an oral glucose load was impaired, leading to higher blood glucose levels (Miyawaki, Yamada et al. 1999).

In a further embodiment, the medicament comprises a further pharmaceutically active agent. Such further pharmaceutically active compound is, among others but not limited thereto, a compound for treatment and/or prevention of diabetes, preferably DM2, and of diabetic complications, whereby the compound is selected from the group comprising, sulfonylurea drugs, biguanides, alpha-glucosidase inhibitors, thiazolinediones, DPP4 inhibitors, meglititinides, glucagon-like peptide analogs, gastric inhibitory peptide analogs, amylin analogs, incretin mimetics, insulin and other therapeutics used in the treatment of insulin resistance and/or DM2 or used in the prevention of insulin resistance and/or DM2, and the like. It will be understood by the one skilled in the art that given the various indications which can be addressed in accordance with the present invention by the nucleic acid molecule according to the present invention, said further pharmaceutically active agent(s) may be any one which in principle is suitable for the treatment and/or prevention of such diseases. The nucleic acid molecule according to the present invention, particularly if present or used as a medicament, is preferably combined with sulfonylurea drugs, biguanides, alpha-glucosidase inhibitors, thiazolinediones, meglitinides, glucagon-like peptide analogs, gastric inhibitory peptide analogs, amylin analogs, incretin mimetics, DPP4 inhibitors, insulin and other therapeutics used in the treatment of DM1, insulin resistance and/or DM2 or used in the prevention of insulin resistance and/or DM2, and the like.

It is within the present invention that the medicament is alternatively or additionally used, in principle, for the prevention of any of the diseases disclosed in connection with the use of the medicament for the treatment of said diseases. Respective markers therefore, i.e. for the respective diseases are known to the ones skilled in the art. Preferably, the respective marker is hyperglucagonemia. Alternatively and/or additionally, the respective marker is selected from the group comprising oxyntomodulin, glicentin, and GIP (for a GIP-binding nucleic acid molecule). A still further group of markers is selected from the group comprising strong thirst, high drinking volume, frequent urination, extreme hungry feeling, HbA1c value, plasma insulin level, plasma glucose level after OGT, fed fasting plasma glucose level, fasting plasma glucose level, urine glucose level, body weight, blood pressure, lassitude, tiredness, weight loss in absence of a diet, weight gain, frequent bacterial or fungal infections, bad wound healing, numbness in hands and feet and impaired vision.

In one embodiment of the medicament of the present invention, such medicament is for use in combination with other treatments for any of the diseases disclosed herein, particularly those for which the medicament of the present invention is to be used.

“Combination therapy” (or “co-therapy”) includes the administration of a medicament of the invention and at least a second or further agent as part of a specific treatment regimen intended to provide at least the beneficial effect from the co-action of these therapeutic agents, i. e. the medicament of the present invention and said second or further agent. The beneficial effect of the combination includes, but is not limited to, pharmacokinetic or pharmacodynamic co-action resulting from the combination of the therapeutically effective agents. Administration of these therapeutically effective agents in combination is typically carried out over a defined time period (usually minutes, hours, days or weeks depending upon the combination selected).

“Combination therapy” may be, but generally is not, intended to encompass the administration of two or more of these therapeutically effective agents as part of separate monotherapy regimens. “Combination therapy” is intended to embrace administration of these therapeutically effective agents in a sequential manner, that is, wherein each therapeutically effective agent is administered at a different time, as well as administration of these therapeutically effective agents, or at least two of the therapeutically effective agents, in a substantially simultaneous manner. Substantially simultaneous administration can be accomplished, for example, by administering to a subject a single capsule having a fixed ratio of each of the therapeutically effective agents or in multiple, single capsules for each of the therapeutically effective agents.

Sequential or substantially simultaneous administration of each therapeutically effective agent can be effected by any appropriate route including, but not limited to, topical routes, oral routes, intravenous routes, intramuscular routes, and direct absorption through mucous membrane tissues. The therapeutic agents can be administered by the same route or by different routes. For example, a first therapeutically effective agent of the combination selected may be administered by injection while the other therapeutically effective agent(s) of the combination may be administered topically.

Alternatively, for example, all therapeutically effective agents may be administered topically or all therapeutically effective agents may be administered by injection. The sequence in which the therapeutically effective agents are administered is not narrowly critical unless noted otherwise. “Combination therapy” also can embrace the administration of the therapeutically effective agents as described above in further combination with other biologically active ingredients. Where the combination therapy further comprises a non-drug treatment, the non-drug treatment may be conducted at any suitable time as long as a beneficial effect from the co-action of the combination of the therapeutically effective agents and non-drug treatment is achieved. For example, in appropriate cases, the beneficial effect is still achieved when the non-drug treatment is temporally removed from the administration of the therapeutically effective agents, perhaps by days or even weeks.

As outlined in general terms above, the medicament according to the present invention can be administered, in principle, in any form known to the ones skilled in the art. A preferred route of administration is systemic administration, more preferably by parenteral administration, preferably by injection. Alternatively, the medicament may be administered locally. Other routes of administration comprise intramuscular, intraperitoneal, and subcutaneous, per orum, intranasal, intratracheal or pulmonary with preference given to the route of administration that is the least invasive, while ensuring efficiency.

Parenteral administration is generally used for subcutaneous, intramuscular or intravenous injections and infusions. Additionally, one approach for parenteral administration employs the implantation of a slow-release or sustained-released systems, which assures that a constant level of dosage is maintained, that are well known to the ordinary skill in the art.

Furthermore, preferred medicaments of the present invention can be administered in intranasal form via topical use of suitable intranasal vehicles, inhalants, or via transdermal routes, using those forms of transdermal skin patches well known to those of ordinary skill in that art. To be administered in the form of a transdermal delivery system, the dosage administration will, of course, be continuous rather than intermittent throughout the dosage regimen. Other preferred topical preparations include creams, ointments, lotions, aerosol sprays and gels.

Subjects that will respond favorably to the method, nucleic acid molecule, pharmaceutical composition and medicament of the invention include medical and veterinary subjects generally, including human beings and human patients. Among others such subject is preferably selected from the group comprising cats, dogs, large animals, avians such as chickens, and the like.

The medicament of the present invention will generally comprise an effective amount of the active component(s) of the therapy, including, but not limited to, a nucleic acid molecule of the present invention, dissolved or dispersed in a pharmaceutically acceptable medium. Pharmaceutically acceptable media or carriers include any and all solvents, dispersion media, coatings, antibacterial and antifungal agents, isotonic and absorption delaying agents and the like. The use of such media and agents for pharmaceutical active substances is well known in the art. Supplementary active ingredients can also be incorporated into the medicament of the present invention.

In a further aspect the present invention is related to a pharmaceutical composition. Such pharmaceutical composition comprises at least one nucleic acid molecule according to the present invention and preferably a pharmaceutically acceptable excipient. Such binder can be any excipient used and/or known in the art. More particularly such excipient is any excipient as discussed in connection with the manufacture of the medicament disclosed herein. In a further embodiment, the pharmaceutical composition comprises a further pharmaceutically active agent.

The preparation of a medicament and a pharmaceutical composition of the invention will be known to those of skill in the art in light of the present disclosure. Typically, such composition may be prepared as an injectable, either as a liquid solution or suspension; a solid form suitable for solution in, or suspension in, liquid prior to injection; as a tablet or any other solid for oral administration; as a time release capsule; or in any other form currently used, including eye drops, a cream, a lotions, a salve, an inhalant and the like. The use of a sterile formulation, such as a saline-based wash, by surgeons, physicians or health care workers to treat a particular area in the operating field may also be particularly useful. Compositions may also be delivered via microdevice, microparticle or sponge.

Upon formulation, a medicament will be administered in a manner compatible with the dosage formulation, and in such amount as is pharmacologically effective. The formulations are easily administered in a variety of dosage forms, such as the type of injectable solutions described above, but drug release capsules and the like can also be employed.

The medicament of the invention can also be administered in oral dosage forms as timed release and sustained release tablets or capsules, pills, powders, granules, elixirs, tinctures, suspensions, syrups and emulsions. Suppositories are advantageously prepared from fatty emulsions or suspensions.

The pharmaceutical composition or medicament may be sterilized and/or contain adjuvants, such as preserving, stabilizing, wetting or emulsifying agents, solution promoters, salts for regulating the osmotic pressure and/or buffers. In addition, they may also contain other therapeutically valuable substances. The compositions are prepared according to conventional techniques including mixing, granulating, or coating methods, and typically contain about 0.1% to 75%, preferably about 1% to 50%, of the active ingredient.

Liquid, particularly injectable compositions can, for example, be prepared by dissolving, dispersing, etc. The active compound is dissolved in or mixed with a pharmaceutically pure solvent such as, for example, water, saline, aqueous dextrose, glycerol, ethanol, and the like, to thereby form the injectable solution or suspension. Additionally, solid forms suitable for dissolving in a liquid prior to injection can be formulated.

The medicaments and nucleic acid molecule, respectively, of the present invention can also be administered in the form of liposomal delivery systems, such as small unilamellar vesicles, large unilamellar vesicles and multilamellar vesicles. Liposomes can be formed from a variety of phospholipids, containing cholesterol, stearylamine or phosphatidylcholines. In some embodiments, a film of lipid components is hydrated with an aqueous solution of drug to form a lipid layer encapsulating the drug, which is well known to the ordinary skilled in the art. For example, the nucleic acid molecule of the invention disclosed herein can be provided as a complex with a lipophilic compound or non-immunogenic, high molecular weight compound constructed using methods known in the art. Additionally, liposomes may bear a nucleic acid molecule of the invention on their surface for targeting and carrying cytotoxic agents internally to mediate cell killing. An example of nucleic-acid associated complexes is provided in U.S. Pat. No. 6,011,020.

The medicaments and nucleic acid molecule, respectively, of the present invention may also be coupled with soluble polymers as targetable drug carriers. Such polymers can include polyvinylpyrrolidone, pyran copolymer, polyhydroxypropyl-methacrylamide-phenol, polyhydroxyethylaspanamidephenol, or polyethyleneoxidepolylysine substituted with palmitoyl residues. Furthermore, the medicaments and nucleic acid molecule, respectively, of the present invention may be coupled to a class of biodegradable polymers useful in achieving controlled release of a drug, for example, polylactic acid, polyepsilon capro lactone, polyhydroxy butyric acid, polyorthoesters, polyacetals, polydihydropyrans, polycyanoacrylates and cross-linked or amphipathic block copolymers of hydrogels.

If desired, the pharmaceutical composition and medicament, respectively, of the invention to be administered may also contain minor amounts of non-toxic auxiliary substances such as wetting or emulsifying agents, pH buffering agents, and other substances such as, for example, sodium acetate, and triethanolamine oleate.

The dosage regimen utilizing the nucleic acid molecules and medicaments, respectively, of the present invention is selected in accordance with a variety of factors including type, species, age, weight, sex and medical condition of the patient; the severity of the condition to be treated; the route of administration; the renal and hepatic function of the patient; and the particular nucleic acid of the invention or salt thereof employed. An ordinarily skilled physician or veterinary can readily determine and prescribe the effective amount of the drug required to prevent, counter or arrest the progress of the condition.

Effective plasma levels of the nucleic acid according to the present invention preferably range from 500 fM to 200 μM, preferably from 1 nM to 20 μM, more preferably from 5 nM to 20 μM, most preferably 50 nM to 20 μM in the treatment of any of the diseases disclosed herein.

The nucleic acid molecule and medicament, respectively, of the present invention may preferably be administered in a single daily dose, every second or third day, weekly, every second week, in a single monthly dose or every third month.

It is within the present invention that the medicament as described herein constitutes the pharmaceutical composition disclosed herein.

In a further aspect the present invention is related to a method for the treatment of a subject who is in need of such treatment, whereby the method comprises the administration of a pharmaceutically active amount of at least one species of the nucleic acid molecule of the present invention. In an embodiment, the subject suffers from a disease or is at risk to develop such disease, whereby the disease is any of those disclosed herein, particularly any of those diseases disclosed in connection with the use of any of the nucleic acid molecule according to the present invention for the manufacture of a medicament.

It is to be understood that the nucleic acid as well as the antagonists according to the present invention can be used not only as a medicament or for the manufacture of a medicament, but also for cosmetic purposes, particularly with regard to the involvement of glucagon in inflamed regional skin lesions.

As preferably used herein a diagnostic or diagnostic agent or diagnostic means—with all three terms being used in an interchangeable manner if not indicated to the contrary—is suitable to detect, either directly or indirectly, glucagon, preferably glucagon as described herein and more preferably glucagon as described herein in connection with the various disorders and diseases described herein. The diagnostic is suitable for the detection and/or follow-up of any of the disorders and diseases, respectively, described herein. Such detection is possible through the binding of a nucleic acid molecule according to the present invention to glucagon. Such binding can be either directly or indirectly be detected. The respective methods and means are known to the ones skilled in the art. Among others, the nucleic acid molecule according to the present invention may comprise a label which allows the detection of the nucleic acids molecule according to the present invention, preferably the nucleic acid bound to glucagon. Such a label is preferably selected from the group comprising radioactive, enzymatic and fluorescent labels. In principle, all known assays developed for antibodies can be adopted for the nucleic acid molecule according to the present invention whereas the target-binding antibody is substituted to a target-binding nucleic acid. In antibody-assays using unlabeled target-binding antibodies the detection is preferably done by a secondary antibody which is modified with radioactive, enzymatic and fluorescent labels and bind to the target-binding antibody at its Fc-fragment. In the case of a nucleic acid molecule, preferably a nucleic acid molecule according to the present invention, the nucleic acid molecule is modified with such a label, whereby preferably such a label is selected from the group comprising biotin, Cy-3 and Cy-5, and such label is detected by an antibody directed against such label, e.g. an anti-biotin antibody, an anti-Cy3 antibody or an anti-Cy5 antibody, or—in the case that the label is biotin—the label is detected by streptavidin or avidin which naturally binds to biotin. Such antibody, streptavidin or avidin in turn is preferably modified with a respective label, e.g. a radioactive, enzymatic or fluorescent label (like an secondary antibody).

In a further embodiment the nucleic acid molecule according to the invention is detected or analysed by a second detection means, wherein the said detection means is a molecular beacon. The methodology of molecular beacon is known to persons skilled in the art and reviewed by Mairal et al. (Mairal et al., 2008).

It will be acknowledged that the detection of glucagon using a nucleic acid molecule according to the present invention will particularly allow the detection of glucagon as defined herein.

In connection with the detection of glucagon a preferred method comprises the following steps:
(a) providing a sample which is to be tested for the presence of glucagon,
(b) providing a nucleic acid molecule according to the present invention,
(c) reacting the sample with the nucleic acid molecule, preferably in a reaction vessel
whereby step (a) can be performed prior to step (b), or step (b) can be preformed prior to step (a).

In a preferred embodiment a further step d) is provided, which consists in the detection of the reaction of the sample with the nucleic acid molecule. Preferably, the nucleic acid molecule of step b) is immobilised to a surface. The surface may be the surface of a reaction vessel such as a reaction tube, a well of a plate, or the surface of a device contained in such reaction vessel such as, for example, a bead. The immobilisation of the nucleic acid molecule to the surface can be made by any means known to the ones skilled in the art including, but not limited to, non-covalent or covalent linkages. Preferably, the linkage is established via a covalent chemical bond between the surface and the nucleic acid molecule. However, it is also within the present invention that the nucleic acid molecule is indirectly immobilised to a surface, whereby such indirect immobilisation involves the use of a further component or a pair of interaction partners. Such further component is preferably a compound which specifically interacts with the nucleic acid molecule to be immobilised which is also referred to as interaction partner, and thus mediates the attachment of the nucleic acid molecule to the surface. The interaction partner is preferably selected from the group comprising nucleic acids, polypeptides, proteins and antibodies. Preferably, the interaction partner is an antibody, more preferably a monoclonal antibody. Alternatively, the interaction partner is a nucleic acid molecule, preferably a functional nucleic acid molecule. More preferably such functional nucleic acid molecule is selected from the group comprising an aptamer, a spiegelmer, and a nucleic acid molecule which is at least partially complementary to the nucleic acid molecule. In a further alternative embodiment, the binding of the nucleic acid molecule to the surface is mediated by a multi-partite interaction partner. Such multi-partite interaction partner is preferably a pair of interaction partners or an interaction partner consisting of a first member and a second member, whereby the first member is comprised by or attached to the nucleic acid molecule and the second member is attached to or comprised by the surface. The multi-partite interaction partner is preferably selected from the group of pairs of interaction partners comprising biotin and avidin, biotin and streptavidin, and biotin and neutravidin. Preferably, the first member of the pair of interaction partners is biotin.

A preferred result of such method is the formation of an immobilised complex of glucagon and the nucleic acid molecule, whereby more preferably said complex is detected. It is within an embodiment that from the complex the glucagon is detected.

A respective detection means which is in compliance with this requirement is, for example, any detection means which is specific for that/those part(s) of the glucagon. A particularly preferred detection means is a detection means which is selected from the group comprising a nucleic acid molecule, a polypeptide, a protein and an antibody, the generation of which is known to the ones skilled in the art.

The method for the detection of glucagon also comprises that the sample is removed from the reaction vessel which has preferably been used to perform step c).

The method comprises in a further embodiment also the step of immobilising an interaction partner of glucagon on a surface, preferably a surface as defined above, whereby the interaction partner is defined as herein and preferably as above in connection with the respective method and more preferably comprises a nucleic acid molecule, a polypeptide, a protein and an antibody in their various embodiments. In this embodiment, a particularly preferred detection means is a nucleic acid molecule according to the present invention, whereby such nucleic acid molecule may preferably be labelled or non-labelled. In case such nucleic acid molecule is labelled it can directly or indirectly be detected. Such detection may also involve the use of a second detection means which is, preferably, also selected from the group comprising a nucleic acid molecule, a polypeptide and a protein described herein. Such detection means are preferably specific for the nucleic acid molecule according to the present invention. In a more preferred embodiment, the second detection means is a molecular beacon. Either the nucleic acid molecule or the second detection means or both may comprise in a preferred embodiment a detection label. The detection label is preferably selected from the group comprising biotin, a bromo-desoxyuridine label, a digoxigenin label, a fluorescence label, a UV-label, a radio-label, and a chelator molecule. Alternatively, the second detection means interacts with the detection label which is preferably contained by, comprised by or attached to the nucleic acid. Particularly preferred combinations are as follows:
the detection label is biotin and the second detection means is an antibody directed against biotin, or wherein
the detection label is biotin and the second detection means is an avidin or an avidin carrying molecule, or wherein
the detection label is biotin and the second detection means is a streptavidin or a stretavidin carrying molecule, or wherein
the detection label is biotin and the second detection means is a neutravidin or a neutravidin carrying molecule, or
wherein the detection label is a bromo-desoxyuridine and the second detection means is an antibody directed against bromo-desoxyuridine, or wherein
the detection label is a digoxigenin and the second detection means is an antibody directed against digoxigenin, or wherein
the detection label is a chelator and the second detection means is a radio-nuclide, whereby it is preferred that said detection label is attached to the nucleic acid molecule. It is to be acknowledged that this kind of combination is also applicable to the embodiment where the nucleic acid molecule is attached to the surface. In such embodiment it is preferred that the detection label is attached to the interaction partner.

Finally, it is also within the present invention that the second detection means is detected using a third detection means, preferably the third detection means is an enzyme, more preferably showing an enzymatic reaction upon detection of the second detection means, or the third detection means is a means for detecting radiation, more preferably radiation emitted by a radio-nuclide. Preferably, the third detection means is specifically detecting and/or interacting with the second detection means.

Also in the embodiment with an interaction partner of glucagon being immobilised on a surface and the nucleic acid molecule according to the present invention being preferably added to the complex formed between the interaction partner and the glucagon, the sample can be removed from the reaction, more preferably from the reaction vessel where step c) and/or d) are preformed.

In an embodiment the nucleic acid molecule according to the present invention comprises a fluorescence moiety and whereby the fluorescence of the fluorescence moiety is different upon complex formation between the nucleic acid molecule and glucagon and free glucagon.

In a further embodiment the nucleic acid molecule is a derivative of the nucleic acid molecule according to the present invention, whereby the derivative of the nucleic acid molecule comprises at least one fluorescent derivative of adenosine replacing adenosine. In a preferred embodiment the fluorescent derivative of adenosine is ethenoadenosine.

In a further embodiment the complex consisting of the derivative of the nucleic acid molecule according to the present invention and the glucagon is detected using fluorescence.

In an embodiment of the method a signal is created in step (c) or step (d) and preferably the signal is correlated with the concentration of glucagon in the sample.

In a preferred embodiment, the assays may be performed in 96-well plates, where components are immobilized in the reaction vessels as described above and the wells acting as reaction vessels.

The nucleic acid molecule of the invention may be further used as starting material for drug discovery. Basically, there are two possible approaches. One approach is the screening of compound libraries whereas such compound libraries are preferably low molecular weight compound libraries. In an embodiment thereof, the screening is a high throughput screening. Preferably, high throughput screening is the fast, efficient, trial-and-error evaluation of compounds in a target based assay. In best case the analysis are carried by a colorimetric measurement. Libraries as used in connection therewith are known to the one skilled in the art.

In case of screening of compound libraries, such as by using a competitive assay which are known to the one skilled in the arts, appropriate glucagon analogues, glucagon: agonists or glucagon antagonists may be found. Such competitive assays may be set up as follows. A nucleic acid molecule of the invention, preferably a spiegelmer, i.e. an L-nucleic acid of the invention, is coupled to a solid phase. In order to identify glucagon analogues labelled glucagon may be added to the assay. A potential analogue would compete with the glucagon molecules binding to the nucleic acid molecule of the invention which would go along with a decrease in the signal obtained by the respective label. Screening for agonists or antagonists may involve the use of a cell culture assay as known to the ones skilled in the art.

The kit according to the present invention may comprise at least one or several of the species of the nucleic acid molecule of the invention, preferably for the detection of a glucagon, more preferably for the detection of glucagon. Additionally, the kit may comprise at least one or several positive or negative controls. A positive control may, for example, be glucagon, particularly the one against which the nucleic acid molecule of the invention is selected or to which it binds, preferably, in liquid form. A negative control may, e.g., be a peptide which is defined in terms of biophysical properties similar to glucagon but which is not recognized by the nucleic acid nucleic acid molecule of the invention. Furthermore, said kit may comprise one or several buffers. The various ingredients may be contained in the kit in dried or lyophilised form or solved in a liquid. The kit may comprise one or several containers which in turn may contain one or several ingredients of the kit. In a further embodiment, the kit comprises an instruction or instruction leaflet which provides to the user information on how to use the kit and its various ingredients.

The pharmaceutical and bioanalytical determination of the nucleic acid according to the present invention is important for the assessment of its pharmacokinetic and biodynamic profile in several humors, tissues and organs of the human and non-human body. For such purpose, any of the detection methods disclosed herein or known to a person skilled in the art may be used. In a further aspect of the present invention a sandwich hybridisation assay for the detection of the nucleic acid molecule according to the present invention is provided. Within the detection assay a capture probe and a detection probe are used. The capture probe is complementary to the first part and the detection probe to the second part of the nucleic acid molecule according to the present invention. The capture probe is immobilised to a surface or matrix. The detection probe preferably carries a marker molecule or label that can be detected as previously described herein.

The detection of the nucleic acid molecule according to the present invention can be carried out as follows:

The nucleic acid molecule according to the present invention hybridises with one of its ends to the capture probe and with the other end to the detection probe. Afterwards, unbound detection probe is removed by, e. g., one or several washing steps. The amount of bound detection probe which preferably carries a label or marker molecule can be measured subsequently as, for example, outlined in more detail in WO/2008/052774 which is incorporated herein by reference.

As preferably used herein, the term treatment comprises in a preferred embodiment additionally or alternatively prevention and/or follow-up.

As preferably used herein, the terms disease and disorder shall be used in an interchangeable manner, if not indicated to the contrary.

As used herein, the term comprise is preferably not intended to limit the subject matter followed or described by such term. However, in an alternative embodiment the term comprises shall be understood in the meaning of containing and thus as limiting the subject matter followed or described by such term.

The various SEQ ID NOs:, the chemical nature of the nucleic acid molecules according to the present invention, the actual sequence thereof and the internal reference number is summarized in the following table.

TABLE 1

| SEQ ID | | | |
|---|---|---|---|
| NO: | | Sequence | Internal Reference |
| 1 | L-DNA | GCACTGGTGAAATGGGAGGGCTAGGTGGAAGGAATCTGAGGCAGTGC | 257-A1-001 |
| 2 | L-DNA | GCACTGGTGAAATGGGAGGGCTATGTGGAAGGAATCTGAGGCAGTGC | 257-D4-001 |
| 3 | L-DNA | GCACTGATGAAATGGGAGGGCTAGGTGGAAGGAATCTGAAGCAGTGC | 257-F4-001 |
| 4 | L-DNA | GCACTAGGGAAATGGGAGGGCTAGGCGGAAGGAATCTGAGGTAGTGC | 257-B3-001 |
| 5 | L-DNA | GCACTAACGAAATGGGAGGGCTAGGTGGAAGGAATCTAAGGTAGTGC | 257-D3-001 |
| 6 | L-DNA | GCAGTGGCGAAATGGGAGGGCTAGGTGGAAGGAATCTGAGTCACTGC | 257-E4-001 |
| 7 | L-DNA | GCAGTGGGGAAATGGGAGGGCTAGGTGGAAGGAATCTGAGCTACTGC | 257-E1-001 |
| 8 | L-DNA | GCATTACTGAAATGGGAGGGCTAGGTGGAAGGAATCTGGAGTAATGC | 257-C4-001 |
| 9 | L-DNA | GCGCTGGGGAAATGGGAGGGCTAGGTGGAAGGAATCTGAGGCAGTGC | 257-C1-001 |
| 10 | L-DNA | GCGCCAGCGAAATGGGAGGGCTAGGTGGAAGGAATCTGAGTCGGCGC | 257-H2-001 |
| 11 | L-DNA | CAGTGGGGAAATGGGAGGGCTAGGTGGAAGGAATCTGAGCTACTG | 257-E1-002 |
| 12 | L-DNA | GAGTGGGGAAATGGGAGGGCTAGGTGGAAGGAATCTGAGCTACTC | 257-E1-003 |
| 13 | L-DNA | AGTGGGGAAATGGGAGGGCTAGGTGGAAGGAATCTGAGCTACT | 257-E1-004 |
| 14 | L-DNA | GTGGGGAAATGGGAGGGCTAGGTGGAAGGAATCTGAGCTAC | 257-E1-005 |
| 15 | L-DNA/L-RNA | GCAGTGGGGAAATGrGGAGGGCTAGGTGGAAGGAATCTGAGCTACTGC | 257-E1-R15-001 |
| 16 | L-DNA/L-RNA | GCAGTGGGGAAATGGGAGGGCTAGGTGGrAAGGAATCTGAGCTACTGC | 257-E1-R29-001 |
| 17 | L-DNA/L-RNA | GCAGTGGGGAAATGGGAGGGCTAGGTGGArAGGAATCTGAGCTACTGC | 257-E1-R30-001 |
| 18 | L-DNA/L-RNA | GCAGTGGGGAAATGrGGAGGGCTAGGTGGrAAGGAATCTGAGCTACTGC | 257-E1-R15/29-001 |
| 19 | L-DNA/L-RNA | GCAGTGGGGAAATGGGAGGGCTAGGTGGrArAGGAATCTGAGCTACTGC | 257-E1-R29/30-001 |
| 20 | L-DNA/L-RNA | GCAGTGGGGAAATGrGGAGGGCTAGGTGGrArAGGAATCTGAGCTACTGC | 257-E1-R15/29/30-001 |
| 21 | L-DNA/L-RNA | GCAGTGGGGAAATGGGArGGGCTAGGTGGrArAGGAATCTGAGCTACTGC | 257-E1-R18/29/30-001 |
| 22 | L-DNA/L-RNA | GCAGTGGGGAAATGrGGArGGGCTAGGTGGrArAGGAATCTGAGCTACTGC | 257-E1-R15/18/29/30-001 |
| 23 | L-DNA/L-RNA | GCAGTGGGrGAAATGrGGArGrGGCTAGGTGGrArAGGAATCTGAGCTACTGC | 257-E1-6xR-001 |
| 24 | L-DNA/L-RNA | GAGTGGGrGAAATGrGGArGrGGCTAGGTGGrArAGGAATCTGAGCTACTC | 257-E1-6xR-003 |
| 25 | L-DNA/L-RNA | AGTGGGrGAAATGrGGArGrGGCTAGGTGGrArAGGAATCTGAGCTACT | 257-E1-6xR-004 |
| 26 | L-DNA/L-RNA | GGGTGGGrGAAATGrGGArGrGGCTAGGTGGrArAGGAATCTGAGCTACCC | 257-E1-6xR-005 |
| 27 | L-DNA/L-RNA | GCGTGGGrGAAATGrGGArGrGGCTAGGTGGrArAGGAATCTGAGCTACGC | 257-E1-6xR-006 |
| 28 | L-DNA/L-RNA | GGGCGGGrGAAATGrGGArGrGGCTAGGTGGrArAGGAATCTGAGCTGCCC | 257-E1-6xR-007 |
| 29 | L-DNA/L-RNA | GCGCGGGrGAAATGrGGArGrGGCTAGGTGGrArAGGAATCTGAGCTGCGC | 257-E1-6xR-008 |
| 30 | L-DNA/L-RNA | GGGCGGGrGAAATGrGGArGrGGCTAGGTGGrArAGGAATCTGAGCCGCCC | 257-E1-6xR-009 |
| 31 | L-DNA/L-RNA | GCGCGGGrGAAATGrGGArGrGGCTAGGTGGrArAGGAATCTGAGCCGCGC | 257-E1-6xR-010 |
| 32 | L-DNA/L-RNA | GGGCCGGrGAAATGrGGArGrGGCTAGGTGGrArAGGAATCTGAGCGGCCC | 257-E1-6xR-011 |
| 33 | L-DNA/L-RNA | GCGCCGGrGAAATGrGGArGrGGCTAGGTGGrArAGGAATCTGAGCGGCGC | 257-E1-6xR-012 |
| 34 | L-DNA/L-RNA | GAGCGGGrGAAATGrGGArGrGGCTAGGTGGrArAGGAATCTGAGCCGCTC | 257-E1-6xR-013 |
| 35 | L-DNA/L-RNA | GAGCCGGrGAAATGrGGArGrGGCTAGGTGGrArAGGAATCTGAGCGGCTC | 257-E1-6xR-014 |
| 36 | L-DNA/L-RNA | GAGTGGGrGAAATGrGGArGrGGCTAGGTGGrArAGGAATCTGAGCCACTC | 257-E1-6xR-015 |
| 37 | L-DNA/L-RNA | GCGTGGGrGAAATGrGGArGrGGCTAGGTGGrArAGGAATCTGAGCCACGC | 257-E1-6xR-016 |
| 38 | L-DNA/L-RNA | GAGTCGGrGAAATGrGGArGrGGCTAGGTGGrArAGGAATCTGAGCGACTC | 257-E1-6xR-017 |
| 39 | L-DNA/L-RNA | GCGTCGGrGAAATGrGGArGrGGCTAGGTGGrArAGGAATCTGAGCGACGC | 257-E1-6xR-018 |
| 40 | L-DNA/L-RNA | GGCGGGrGAAATGrGGArGrGGCTAGGTGGrArAGGAATCTGAGCCGCC | 257-E1-6xR-019 |
| 41 | L-DNA/L-RNA | CGCGGGrGAAATGrGGArGrGGCTAGGTGGrArAGGAATCTGAGCCGCG | 257-E1-6xR-020 |
| 42 | L-DNA/L-RNA | GCGGGrGAAATGrGGArGrGGCTAGGTGGrArAGGAATCTGAGCCGC | 257-E1-6xR-029 |
| 43 | L-DNA/L-RNA | GCGGrGAAATGrGGArGrGGCTAGGTGGrArAGGAATCTGAGCGC | 257-E1-6xR-030 |
| 44 | L-DNA/L-RNA | CGGGrGAAATGrGGArGrGGCTAGGTGGrArAGGAATCTGAGCCG | 257-E1-6xR-031 |
| 45 | L-DNA/L-RNA | GGGrGAAATGrGGArGrGGCTAGGTGGrArAGGAATCTGAGCC | 257-E1-6xR-032 |
| 46 | L-DNA/L-RNA | GGrGAAATGrGGArGrGGCTAGGTGGrArAGGAATCTGAGC | 257-E1-6xR-033 |
| 47 | L-DNA/L-RNA | GCGCGGGrGAAATGrGGArGrGGCTAGGrTGGrArAGGAATCTGAGCCGCGC | 257-E1-7xR-023 |
| 48 | L-DNA/L-RNA | GCGGrGAAATGrGGArGrGGCTAGGrTGGrArAGGAATCTGAGCGC | 257-E1-7xR-037 |
| 49 | L-DNA | CGACTCGAGAGGAAAGGTTGCTAAAGGTTCGGTTGGATTCACTCGAGTCG | 259-D5-001 |
| 50 | L-DNA | CGACTCGAGAGGAAAGGTTGGTAAAGGTTCGGTTGGATTCACTCGAGTCG | 259-H6-001 |
| 51 | L-DNA | CGACTCGAGAGGAAAGGTTGGTATAGGTTCGGTTGGATTCACTCGAGTCG | 259-B7-001 |
| 52 | L-DNA | CGACTCGAGAGGAAATGTTGGTAAAGGTTCGGTTGGATTCACTCGAGTCG | 259-B8-001 |
| 53 | L-DNA | CGACTCGAGAGGAGAGGTTGGTAAAGATTCGGTTGGATTCACTCGAGTCG | 259-A5-001 |
| 54 | L-DNA | CGGCTCGAGAGGAAAGGTTGGTAAAGGTTCGGTTGGATTCACTCGAGTCG | 259-C8-001 |
| 55 | L-DNA | CGACTCGAGATGAAAGGTTGGCAAAGGTTCGGTTGGATTCACTCGAGTCG | 259-E5-001 |
| 56 | L-DNA | CGAGTCGATAGAAGGTCGGTAAGTTTCGGTAGGATCTGCGACGAGACG | 259-E7-001 |
| 57 | L-DNA | CGAGTCGATAGAAGGTTGGTAAGTTTCGGTTGGATCTGCGACGAGACG | 259-F5-001 |
| 58 | L-DNA | ACTCGAGAGGAAAGGTTGGTAAAGGTTCGGTTGGATTCACTCGAGT | 259-H6-002 |
| 59 | L-DNA | GTCGAGAGGAAAGGTTGGTAAAGGTTCGGTTGGATTCACTCGAC | 259-H6-005 |
| 60 | L-DNA | TCGAGAGGAAAGGTTGGTAAAGGTTCGGTTGGATTCACTCGA | 259-H6-003 |
| 61 | L-DNA | GAGAGGAAAGGTTGGTAAAGGTTCGGTTGGATTCACTC | 259-H6-004 |
| 62 | L-DNA | ACTCGAGAGGAAGGTTGGTAAAGGTTCGGTTGGATTCACTCGAGT | 259-H6-006 |
| 63 | L-DNA | ACTCGAGAGGAAAGGTTGGTAAGGTTCGGTTGGATTCACTCGAGT | 259-H6-007 |
| 64 | L-DNA | ACTCGAGAGGAAGGTTGGTAAGGTTCGGTTGGATTCACTCGAGT | 259-H6-008 |
| 65 | L-DNA/L-RNA | ACTCGAGAGGAArAGGTTGGTAAAGGTTCGGTTGGATTCACTCGAGT | 259-H6-002-R13 |
| 66 | L-DNA/L-RNA | ACTCGAGAGGAAAGGTTGGTAAArGGTTCGGTTGGATTCACTCGAGT | 259-H6-002-R24 |
| 67 | L-DNA/L-RNA | ACTCGAGAGGAAAGGTTGGTAAAGGTTCGGTTGGArUTCACTCGAGT | 259-H6-002-R36 |
| 68 | L-DNA/L-RNA | ACTCGAGAGGAArAGGTTGGTAAArGGTTCGGTTGGATTCACTCGAGT | 259-H6-002-R13/24 |
| 69 | L-DNA/L-RNA | ACTCGAGAGGAArAGGTTGGTAAAGGTTCGGTTGGArUTCACTCGAGT | 259-H6-002-R13/36 |
| 70 | L-DNA/L-RNA | ACTCGAGAGGAAAGGTTGGTAAArGGTTCGGTTGGArUTCACTCGAGT | 259-H6-002-R24/36 |
| 71 | L-DNA/L-RNA | ACTCGAGAGGAArAGGTTGGTAAArGGTTCGGTTGGArUTCACTCGAGT | 259-H6-002-R13/24/36 |
| 72 | L-DNA/L-RNA | GTCGAGAGGAArAGGTTGGTAAAGGTTCGGTTGGATTCACTCGAC | 259-H6-005-R12 |
| 73 | L-DNA/L-RNA | TTCGAGAGGAArAGGTTGGTAAAGGTTCGGTTGGATTCACTCGAA | 259-H6-009-R12 |
| 74 | L-DNA/L-RNA | TGCGAGAGGAArAGGTTGGTAAAGGTTCGGTTGGATTCACTCGCA | 259-H6-010-R12 |
| 75 | L-DNA/L-RNA | GGCGAGAGGAArAGGTTGGTAAAGGTTCGGTTGGATTCACTCGCC | 259-H6-011-R12 |
| 76 | L-DNA/L-RNA | GGCCAGAGGAArAGGTTGGTAAAGGTTCGGTTGGATTCACTGGCC | 259-H6-012-R12 |
| 77 | L-DNA/L-RNA | GCGCAGAGGAArAGGTTGGTAAAGGTTCGGTTGGATTCACTGCGC | 259-H6-013-R12 |
| 78 | L-DNA/L-RNA | GCCGAGAGGAArAGGTTGGTAAAGGTTCGGTTGGATTCACTCGGC | 259-H6-014-R12 |
| 79 | L-DNA/L-RNA | CTCGAGAGGAArAGGTTGGTAAAGGTTCGGTTGGATTCACTCGAG | 259-H6-015-R12 |
| 80 | L-DNA/L-RNA | CTCGAGAGGAArAGGTTGGTAAAGGTTCGGTTGGATTCACTCGAGT | 259-H6-016-R12 |
| 81 | L-DNA/L-RNA | GCCGAGAGGAArAGGTTGGTAAArGGTTCGGTTGGArUTCACTCGGC | 259-H6-014-R12/23/35 |
| 82 | L-DNA/L-RNA | GCCGAGAGGAArAGGTTGGTAAArGGTTCGrGTTGGArUTCrACTCGGC | 259-H6-014-R12/23/29/35/38 |
| 83 | L-DNA | CGGCCTAGAAGGTAGGTAAGTTTCGGTTGGATCTACGGTCGTAACACG | 258-D4-001 |
| 84 | L-DNA | CGTCCTAGAAGGTAGGTAAGTTTCGGTTGGATCTAGGATAGTAGCACG | 258-H1-001 |
| 85 | L-RNA | CGUGUGUGGGUAGAUGCACCUGCGAUUCGCUAAAAAGUGCCACACG | GLU-18-25-A3-001 |
| 86 | L-RNA | CGACGUGUGUGGGUAGAUGCACCUGCGAUUCGCUAAAAAGUGCCACACGUCG | GLU-18-25-A3-002 |
| 87 | L-RNA | CAGACGUGUGUGGGUAGAUGCACCUGCGAUUCGCUAAAAAGUGCCACACGUCUG | NOX-G11stabi2 = |
| | | | GLU-18-25-A3-003 |
| 88 | L-DNA | 5′-40kDa-PEG- | NOX-G12 = 259- |
| | | ACTCGAGAGGAAAGGTTGGTAAAGGTTCGGTTGGATTCACTCGAGT | H6-002-5′-PEG |
| 89 | L-DNA/L-RNA | 5′-40kDa-PEG- | NOX-G13 = 259- |
| | | ACTCGAGAGGAArAGGTTGGTAAAGGTTCGGTTGGATTCACTCGAGT | H6-002-R13-5′-PEG |
| 90 | L-DNA/L-RNA | 5′-40kDa-PEG- | NOX-G14 = 259-H6-014- |
| | | GCCGAGAGGAArAGGTTGGTAAArGGTTCGGTTGGArUTCACTCGGC | R12/23/35-5′-PEG |
| 91 | L-DNA/L-RNA | 5′-40kDa-PEG- | NOX-G15 = 257-E1- |
| | | GCGGrGAAATGrGGArGrGGCTAGGTGGrArAGGAATCTGAGCGC | 6xR-030-5′-PEG |
| 92 | L-DNA/L-RNA | 5′-40kDa-PEG- | NOX-G16 = 257-E1- |
| | | GCGGrGAAATGrGGArGrGGCTAGGrTGGrArAGGAATCTGAGCGC | 7xR-037-5′-PEG |
| 93 | L-DNA/L-RNA | GCAGTGGGrGAAATGGGAGGGCTAGGTGGAAGGAATCTGAGCTACTGC | 257-E1-R9-001 |
| 94 | L-DNA/L-RNA | GCAGTGGGGAAATGGGArGGGCTAGGTGGAAGGAATCTGAGCTACTGC | 257-E1-R18-001 |
| 95 | L-DNA/L-RNA | GCAGTGGGGAAATGGGAGrGGCTAGGTGGAAGGAATCTGAGCTACTGC | 257-E1-R19-001 |
| 96 | L-RNA/L-DNA | CAGAdCGUGUGUGGGUAGAUGCACCUGCGAUUCGCUAAAAAGUGCCACACGUCUG | NOX-G11-D05 |
| 97 | L-RNA/L-DNA | CAGACGdTGUGUGGGUAGAUGCACCUGCGAUUCGCUAAAAAGUGCCACACGUCUG | NOX-G11-D07 |
| 98 | L-RNA/L-DNA | CAGACGUGUGUGGGdTAGAUGCACCUGCGAUUCGCUAAAAAGUGCCACACGUCUG | NOX-G11-D15 |
| 99 | L-RNA/L-DNA | CAGACGUGUGUGGGUdAGAUGCACCUGCGAUUCGCUAAAAAGUGCCACACGUCUG | NOX-G11-D16 |
| 100 | L-RNA/L-DNA | CAGACGUGUGUGGGUAGAdTGCACCUGCGAUUCGCUAAAAAGUGCCACACGUCUG | NOX-G11-D19 |
| 101 | L-RNA/L-DNA | CAGACGUGUGUGGGUAGAUdGCACCUGCGAUUCGCUAAAAAGUGCCACACGUCUG | NOX-G11-D20 |
| 102 | L-RNA/L-DNA | CAGACGUGUGUGGGUAGAUGdCACCUGCGAUUCGCUAAAAAGUGCCACACGUCUG | NOX-G11-D21 |
| 103 | L-RNA/L-DNA | CAGACGUGUGUGGGUAGAUGCdACCUGCGAUUCGCUAAAAAGUGCCACACGUCUG | NOX-G11-D22 |
| 104 | L-RNA/L-DNA | CAGACGUGUGUGGGUAGAUGCAdCCUGCGAUCCGCUAAAAAGUGCCACACGUCUG | NOX-G11-D23 |
| 105 | L-RNA/L-DNA | CAGACGUGUGUGGGUAGAUGCACdCUGCGAUUCGCUAAAAAGUGCCACACGUCUG | NOX-G11-D24 |
| 106 | L-RNA/L-DNA | CAGACGUGUGUGGGUAGAUGCACCdTGCGAUUCGCUAAAAAGUGCCACACGUCUG | NOX-G11-D25 |
| 107 | L-RNA/L-DNA | CAGACGUGUGUGGGUAGAUGCACCUdGCGAUUCGCUAAAAAGUGCCACACGUCUG | NOX-G11-D26 |
| 108 | L-RNA/L-DNA | CAGACGUGUGUGGGUAGAUGCACCUGdCGAUUCGCUAAAAAGUGCCACACGUCUG | NOX-G11-D27 |
| 109 | L-RNA/L-DNA | CAGACGUGUGUGGGUAGAUGCACCUGCGAUUCGCUAAAAAGUGCCdACACGUCUG | NOX-G11-D46 |
| 110 | L-RNA/L-DNA | CAGACGUGUGUGGGUAGAUGCACCUGCGAUUCGCUAAAAAGUGCCACdACGUCUG | NOX-G11-D48 |
| 111 | D-DNA | GCACTGGTGAAATGGGAGGGCTAGGTGGAAGGAATCTGAGGCAGTGC | 257-A1-001 |
| 112 | D-DNA | GCACTGGTGAAATGGGAGGGCTATGTGGAAGGAATCTGAGGCAGTGC | 257-D4-001 |
| 113 | D-DNA | GCACTGATGAAATGGGAGGGCTAGGTGGAAGGAATCTGAAGCAGTGC | 257-F4-001 |
| 114 | D-DNA | GCACTAGGGAAATGGGAGGGCTAGGCGGAAGGAATCTGAGGTAGTGC | 257-B3-001 |
| 115 | D-DNA | GCACTAACGAAATGGGAGGGCTAGGTGGAAGGAATCTAAGGTAGTGC | 257-D3-001 |
| 116 | D-DNA | GCAGTGGCGAAATGGGAGGGCTAGGTGGAAGGAATCTGAGTCACTGC | 257-E4-001 |
| 117 | D-DNA | GCAGTGGGGAAATGGGAGGGCTAGGTGGAAGGAATCTGAGCTACTGC | 257-E1-001 |
| 118 | D-DNA | GCATTACTGAAATGGGAGGGCTAGGTGGAAGGAATCTGGAGTAATGC | 257-C4-001 |
| 119 | D-DNA | GCGCTGGGGAAATGGGAGGGCTAGGTGGAAGGAATCTGAGGCAGTGC | 257-C1-001 |
| 120 | D-DNA | GCGCCAGCGAAATGGGAGGGCTAGGTGGAAGGAATCTGAGTCGGCGC | 257-H2-001 |
| 121 | D-DNA | CAGTGGGGAAATGGGAGGGCTAGGTGGAAGGAATCTGAGCTACTG | 257-E1-002 |
| 122 | D-DNA | GAGTGGGGAAATGGGAGGGCTAGGTGGAAGGAATCTGAGCTACTC | 257-E1-003 |
| 123 | D-DNA | AGTGGGGAAATGGGAGGGCTAGGTGGAAGGAATCTGAGCTACT | 257-E1-004 |
| 124 | D-DNA | GTGGGGAAATGGGAGGGCTAGGTGGAAGGAATCTGAGCTAC | 257-E1-005 |
| 125 | D-DNA | CGACTCGAGAGGAAAGGTTGCTAAAGGTTCGGTTGGATTCACTCGAGTCG | 259-D5-001 |
| 126 | D-DNA | CGACTCGAGAGGAAAGGTTGGTAAAGGTTCGGTTGGATTCACTCGAGTCG | 259-H6-001 |
| 127 | D-DNA | CGACTCGAGAGGAAAGGTTGGTATAGGTTCGGTTGGATTCACTCGAGTCG | 259-B7-001 |
| 128 | D-DNA | CGACTCGAGAGGAAATGTTGGTAAAGGTTCGGTTGGATTCACTCGAGTCG | 259-B8-001 |
| 129 | D-DNA | CGACTCGAGAGGAGAGGTTGGTAAAGATTCGGTTGGATTCACTCGAGTCG | 259-A5-001 |
| 130 | D-DNA | CGGCTCGAGAGGAAAGGTTGGTAAAGGTTCGGTTGGATTCACTCGAGTCG | 259-C8-001 |
| 131 | D-DNA | CGACTCGAGATGAAAGGTTGGCAAAGGTTCGGTTGGATTCACTCGAGTCG | 259-E5-001 |
| 132 | D-DNA | CGAGTCGATAGAAGGTCGGTAAGTTTCGGTAGGATCTGCGACGAGACG | 259-E7-001 |
| 133 | D-DNA | CGAGTCGATAGAAGGTTGGTAAGTTTCGGTTGGATCTGCGACGAGACG | 259-F5-001 |
| 134 | D-DNA | ACTCGAGAGGAAAGGTTGGTAAAGGTTCGGTTGGATTCACTCGAGT | 259-H6-002 |
| 135 | D-DNA | GTCGAGAGGAAAGGTTGGTAAAGGTTCGGTTGGATTCACTCGAC | 259-H6-005 |
| 136 | D-DNA | TCGAGAGGAAAGGTTGGTAAAGGTTCGGTTGGATTCACTCGA | 259-H6-003 |
| 137 | D-DNA | GAGAGGAAAGGTTGGTAAAGGTTCGGTTGGATTCACTC | 259-H6-004 |
| 138 | D-DNA | ACTCGAGAGGAAGGTTGGTAAAGGTTCGGTTGGATTCACTCGAGT | 259-H6-006 |
| 139 | D-DNA | ACTCGAGAGGAAAGGTTGGTAAGGTTCGGTTGGATTCACTCGAGT | 259-H6-007 |
| 140 | D-DNA | ACTCGAGAGGAAGGTTGGTAAGGTTCGGTTGGATTCACTCGAGT | 259-H6-008 |
| 141 | D-DNA/D-RNA | ACTCGAGAGGAArAGGTTGGTAAAGGTTCGGTTGGATTCACTCGAGT | 259-H6-002-R13 |
| 142 | D-DNA/D-RNA | GTCGAGAGGAArAGGTTGGTAAAGGTTCGGTTGGATTCACTCGAC | 259-H6-005-R12 |
| 143 | D-DNA/D-RNA | TTCGAGAGGAArAGGTTGGTAAAGGTTCGGTTGGATTCACTCGAA | 259-H6-009-R12 |
| 144 | D-DNA/D-RNA | TGCGAGAGGAArAGGTTGGTAAAGGTTCGGTTGGATTCACTCGCA | 259-H6-010-R12 |
| 145 | D-DNA/D-RNA | GGCGAGAGGAArAGGTTGGTAAAGGTTCGGTTGGATTCACTCGCC | 259-H6-011-R12 |
| 146 | D-DNA/D-RNA | GGCCAGAGGAArAGGTTGGTAAAGGTTCGGTTGGATTCACTGGCC | 259-H6-012-R12 |
| 147 | D-DNA/D-RNA | GCGCAGAGGAArAGGTTGGTAAAGGTTCGGTTGGATTCACTGCGC | 259-H6-013-R12 |
| 148 | D-DNA/D-RNA | GCCGAGAGGAArAGGTTGGTAAAGGTTCGGTTGGATTCACTCGGC | 259-H6-014-R12 |
| 149 | D-DNA/D-RNA | CTCGAGAGGAArAGGTTGGTAAAGGTTCGGTTGGATTCACTCGAG | 259-H6-015-R12 |
| 150 | D-DNA/D-RNA | CTCGAGAGGAArAGGTTGGTAAAGGTTCGGTTGGATTCACTCGAGT | 259-H6-016-R12 |
| 151 | D-DNA | CGGCCTAGAAGGTAGGTAAGTTTCGGTTGGATCTACGGTCGTAACACG | 258-D4-001 |
| 152 | D-DNA | CGTCCTAGAAGGTAGGTAAGTTTCGGTTGGATCTAGGATAGTAGCACG | 258-H1-001 |
| 152 | D-RNA | CGUGUGUGGGUAGAUGCACCUGCGAUUCGCUAAAAAGUGCCACACG | GLU-18-25-A3-001 |
| 153 | D-RNA | CGACGUGUGUGGGUAGAUGCACCUGCGAUUCGCUAAAAAGUGCCACACGUCG | GLU-18-25-A3-002 |
| 154 | D-RNA | CAGACGUGUGUGGGUAGAUGCACCUGCGAUUCGCUAAAAAGUGCCACACGUCUG | NOX-G11stabi2 = |
| | | | GLU-18-25-A3-003 |
| 155 | L-DNA | 5′-NH₂-C16- | 259-H6-002- |
| | | ACTCGAGAGGAAAGGTTGGTAAAGGTTCGGTTGGATTCACTCGAGT | 5′-Amino |
| 156 | L-DNA/L-RNA | 5′-NH₂-C16- | 259-H6-002- |
| | | ACTCGAGAGGAArAGGTTGGTAAAGGTTCGGTTGGATTCACTCGAGT | R13-5′-Amino |
| 157 | L-DNA/L-RNA | 5′-NH₂-C16- | 259-H6-014- |
| | | GCCGAGAGGAArAGGTTGGTAAArGGTTCGGTTGGArUTCACTCGGC | R12/23/35-5′-Amino |
| 158 | L-DNA/L-RNA | 5′-NH₂-C16- | 257-E1-6xR-030- |
| | | GCGGrGAAATGrGGArGrGGCTAGGTGGrArAGGAATCTGAGCGC | 5′-Amino |
| 159 | L-DNA/L-RNA | 5′-NH₂-C16- | 257-E1-7xR-037- |
| | | GCGGrGAAATGrGGArGrGGCTAGGrTGGrArAGGAATCTGAGCGC | 5′-Amino |
| 160 | L-peptide | RSLQDTEEKSRSFSASQADPLSDPDQMNEDKRHSQGTFTSDYSKYLDSRRAQD | Glicentin |
| | | FVQWLMNTKRNRNNIA | |
| 161 | L-peptide | RSLQDTEEKSRSFSASQADPLSDPDQMNED | GRPP |
| 162 | L-peptide | HSQGTFTSDYSKYLDSRRAQDFVQWLMNTKRNRNNIA | OXY/OXM |
| 163 | L-peptide | HSQGTFTSDYSKYLDSRRAQDFVQWLMNT | Glucagon (human, |
| | | | rat, mouse, |
| | | | squirrel monkey, |
| | | | pig, rabbit, |
| | | | hamster, dog, |
| | | | sheep, chicken, |
| | | | bovine) |
| 164 | L-peptide | HDEFERHAEGTFTSDVSSYLEGQAAKEFIAWLVKGRG | GLP-1 |
| 165 | L-peptide | HAEGTFTSDVSSYLEGQAAKEFIAWLVKGRG | GLP-1(7-37) |
| 166 | L-peptide | HAEGTFTSDVSSYLEGQAAKEFIAWLVKGR | GLP-1(7-36) |
| 167 | L-peptide | HADGSFSDEMNTILDNLAARDFINWLIQTKITD | GLP-2 |
| 168 | L-peptide | YAEGTFISDYSIAMDKIHQQDFVNWLLAQKGKKNDWKHNITQ | GIP |
| 169 | L-peptide | HADGVFTSDFSKLLGQLSAKKYLESLMGKRVSSNISEDPVPV | Intestinal peptide |
| | | | PHV-42/Prepro-VIP |
| | | | (81-122) |
| 170 | L-peptide | HADGVFTSDFSKLLGQLSAKKYLESLM | Intestinal peptide |
| | | | PHM-27 |
| 171 | L-peptide | HSQGTFTSDYSKYLDSRRAQQFLKWLLNV | Glucagon (Guinea |
| | | | pig) |
| 172 | L-peptide | HSQGTFTSDYSKHLDSRYAQEFVQWLMNT | Glucagon |
| | | | (Chinchilla) |
| 173 | L-DNA/L-RNA | Bn₁AAATGn₂GAn₃n₄GCTAKGn₅GGn₆n₇GGAATCTRRR | |
| | | wherein n₁ is G or rG, n₂ is G or rG, n₃ | |
| | | is G or rG, n₄ is G or rG, n₅ is Y or rT, | |
| | | n₆ is A or rA, n₇ is A or rA, | |
| 174 | L-DNA/L-RNA | Bn₁AAATGn₂GAn₃n₄GCTAGGn₅GGn₆n₇GGAATCTGAR | |
| | | wherein n₁ is G or rG, n₂ is G or rG, n₃ | |
| | | is G or rG, n₄ is G or rG, n₅ is T or rT, | |
| | | n₆ is A or rA, n₇ is A or rA, and | |
| 175 | L-DNA/L-RNA | Tn₁AAATGn₂GAn₃n₄GCTAGGn₅GGn₆n₇GGAATCTGAG | |
| | | wherein n₁ is G or rG, n₂ is G or rG, n₃ | |
| | | is G or rG, n₄ is G or rG, n₅ is T or rT, | |
| | | n₆ is A or rA, n₇ is A or rA, and | |
| 176 | L-DNA/L-RNA | Tn₁AAATGn₂GAn₃n₄GCTAGGn₅GGn₆n₇GGAATCTGAA | |
| | | wherein n₁ is G or rG, n₂ is G or rG, n₃ | |
| | | is G or rG, n₄ is G or rG, n₅ is T or rT, | |
| | | n₆ is A or rA, n₇ is A or rA, | |
| 177 | L-DNA/L-RNA | Cn₁AAATGn₂GAn₃n₄GCTAGGn₅GGn₆n₇GGAATCTGAG | |
| | | wherein n₁ is G or rG, n₂ is G or rG, n₃ | |
| | | is G or rG, n₄ is G or rG, n₅ is T or rT, | |
| | | n₆ is A or rA, n₇ is A or rA, | |
| 178 | L-DNA/L-RNA | Gn₁AAATGn₂GAn₃n₄GCTAGGn₅GGn₆n₇GGAATCTGAG | |
| | | wherein n₁ is G or rG, n₂ is G or rG, n₃ | |
| | | is G or rG, n₄ is G or rG, n₅ is T or rT, | |
| | | n₆ is A or rA, n₇ is A or rA, and | |
| 179 | L-DNA/L-RNA | GrGAAATGGGAGGGCTAGGTGGAAGGAATCTGAG | |
| 180 | L-DNA/L-RNA | GGAAATGrGGAGGCTAGGTGGAAGGAATCTGAG | |
| 181 | L-DNA/L-RNA | GGAAATGGGArGGGCTAGGTGGAAGGAATCTGAG | |
| 182 | L-DNA/L-RNA | GGAAATGGGAGrGGCTAGGTGGAAGGAATCTGAG | |
| 183 | L-DNA/L-RNA | GGAAATGGGAGGGCTAGGTGGrAAGGAATCTGAG | |
| 184 | L-DNA/L-RNA | GGAAATGGGAGGGCTAGGTGGArAGGAATCTGAG | |
| 185 | L-DNA/L-RNA | GGAAATGrGGAGGGCTAGGTGGrAAGGAATCTGAG | |
| 186 | L-DNA/L-RNA | GGAAATGGGAGGGCTAGGTGGrArAGGAATCTGAG | |
| 187 | L-DNA/L-RNA | GGAAATGrGGAGGGCTAGGTGGrArAGGAATCTGAG | |
| 188 | L-DNA/L-RNA | GGAAATGGGArGGGCTAGGTGGrArAGGAATCTGAG | |
| 189 | L-DNA/L-RNA | GrGAAATGrGGArGGGCTAGGTGGrArAGGAATCTGAG | |
| 190 | L-DNA/L-RNA | GrGAAATGrGGArGrGGCTAGGTGGrArAGGAATCTGAG | |
| 191 | L-DNA/L-RNA | GrGAAATGrGGArGrGGCTAGGrTGGrArAGGAATCTGAG | |
| 192 | L-DNA | BGAAATGGGAGGGCTAKGYGGAAGGAATCTRRR | |
| 193 | L-DNA | TGAAATGGGAGGGCTAGGTGGAAGGAATCTGAG | |
| 194 | L-DNA | TGAAATGGGAGGGCTAGGTGGAAGGAATCTGAA | |
| 195 | L-DNA | CGAAATGGGAGGGCTAGGTGGAAGGAATCTGAG | |
| 196 | L-DNA | GGAAATGGGAGGGCTAGGTGGAAGGAATCTGAG | |
| 197 | L-DNA/L-RNA | AKGAR n₁KGTTGSYAWAn₂RTTCGn₃TTGGAn₄TCn₅ | |
| | | wherein n₁ is A or rA, n₂ is G or rG, n₃ | |
| | | is G or rG, n₄ is T or rU, n₅ is A or rA, | |
| | | and | |
| 198 | L-DNA | AGAAGGTTGGTAAGTTTCGGTTGGATCTG | |
| 199 | L-DNA | AGAAGGTCGGTAAGTTTCGGTAGGATCTG | |
| 200 | L-DNA | AGGAAGGTTGGTAAAGGTTCGGTTGGATTCA | |
| 201 | L-DNA | AGGAAAGGTTGGTAAGGTTCGGTTGGATTCA | |
| 202 | L-DNA | AGGAAGGTTGGTAAGGTTCGGTTGGATTCA | |
| 203 | L-DNA/L-RNA | AGGAAn₁GGTTGGTAAAn₂GTTCGn₃TTGGAn₄TCn₅ | |
| | | wherein n₁ is A or rA, n₂ is G or rG, n₃ | |
| | | is G or rG, n₄ is T or rU, n₅ is A or rA, | |
| | | and | |
| 204 | L-DNA/L-RNA | AGGAArAGGTTGGTAAAGGTTCGGTTGGATTCA | |
| 205 | L-DNA/L-RNA | AGGAAAGGTTGGTAAArGGTTCGGTTGGATTCA | |
| 206 | L-DNA/L-RNA | AGGAAAGGTTGGTAAAGGTTCGGTTGGArUTCA | |
| 207 | L-DNA/L-RNA | AGGAArAGGTTGGTAAArGGTTCGGTTGGATTCA | |
| 208 | L-DNA/L-RNA | AGGAArAGGTTGGTAAAGGTTCGGTTGGArUTCG | |
| 209 | L-DNA/L-RNA | AGGAArAGGTTGGTAAAGGTTCGGTTGGArUTCA | |
| 210 | L7DNA/L-RNA | AGGAArAGGTTGGTAAArGGTTCGGTTGGArUTCA | |
| 211 | L-DNA/L-RNA | AGGAArAGGTTGGTAAArGGTTCGrGTTGGArUTCrA | |
| 212 | L-DNA | AGGAAAGGTTGGTAAAGGTTCGGTTGGATTCA | |
| 213 | L-DNA | AAGGTTGGTA | |
| 214 | L-DNA | AGGTTCGGTTGGAT | |
| 215 | L-DNA | AGTTTCGGTTGGAT | |
| 216 | L-DNA | AGTTTCGGTAGGAT | |
| 217 | L-DNA | AGTTTCGGTAGGAT | |
| 218 | L-DNA | AGGAAGGTTGGTAAAGGTTCGGTTGGATTCA | |
| 219 | L-DNA | AGGAAAGGTTGGTAAGGTTCGGTTGGATTCA | |
| 220 | L-DNA | AGGAAGGTTGGTAAGGTTCGGTTGGATTCA | |
| 221 | L-DNA | AKGARAKGTTGSYAWAGRTTCGGTTGGATTCA | |

The present invention is further illustrated by the figures, examples and the sequence listing from which further features, embodiments and advantages may be taken, wherein

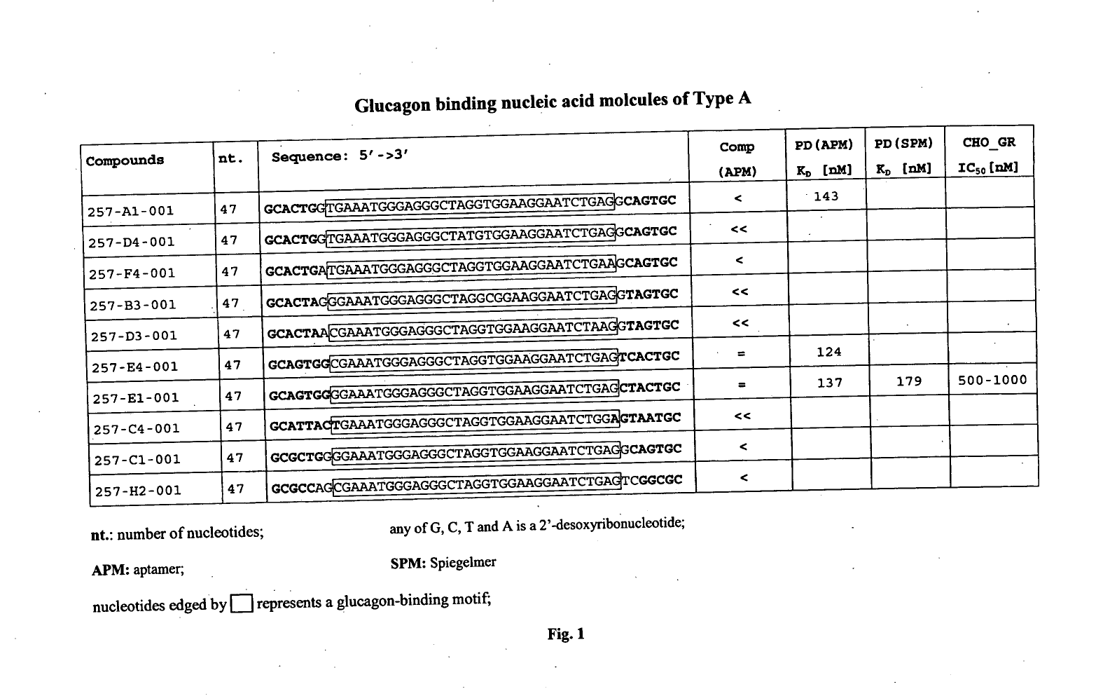

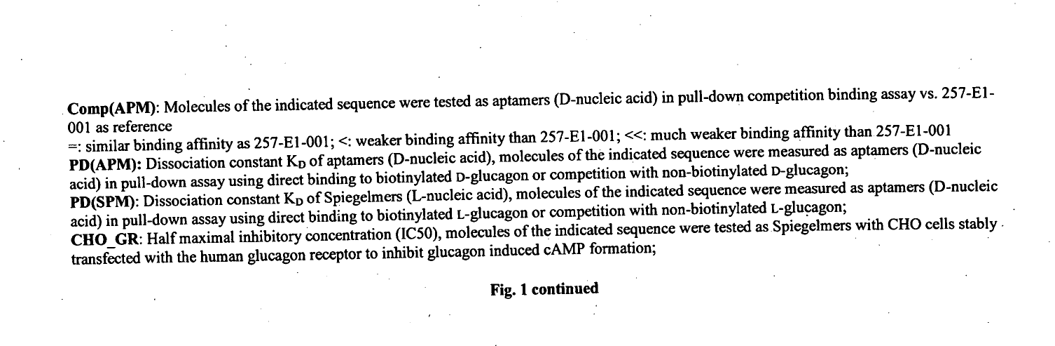

FIG. 1 shows an alignment of sequences of glucagon binding nucleic acid molecules of the invention of “type A”;

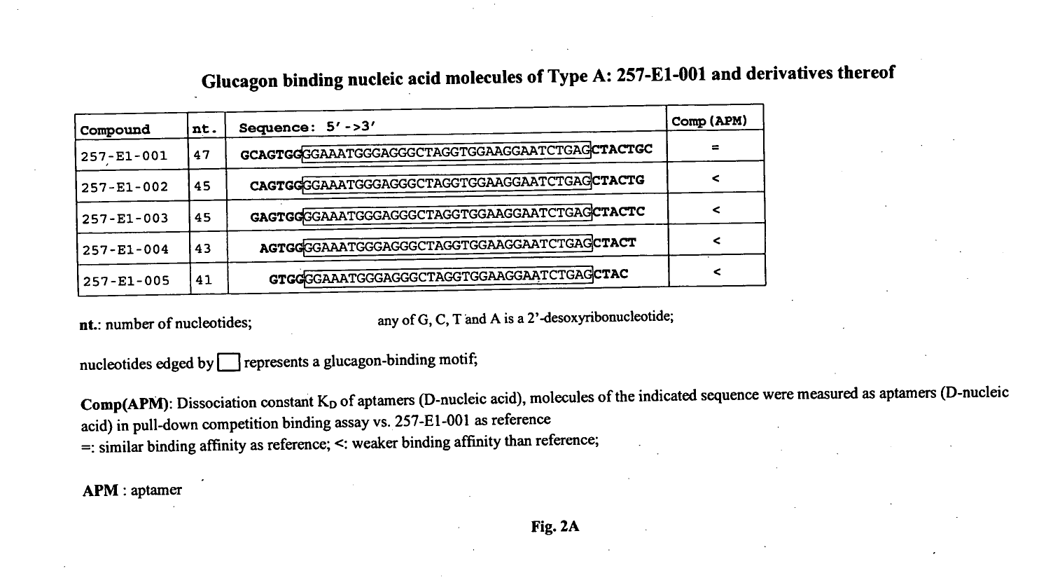

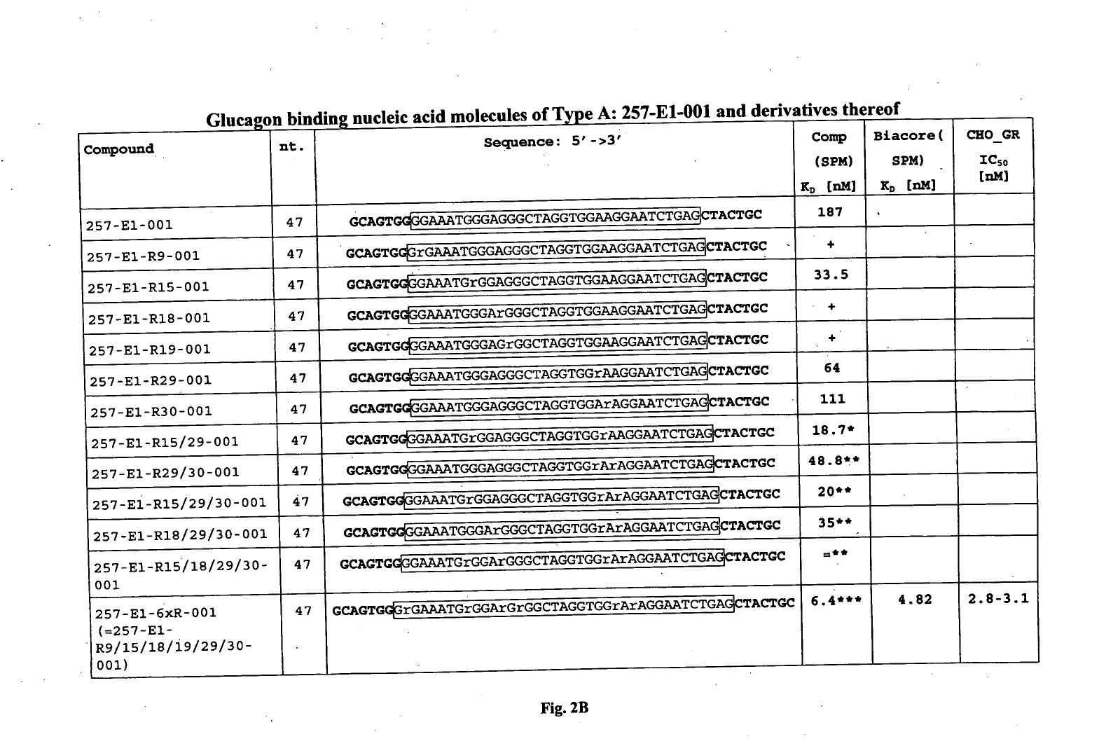

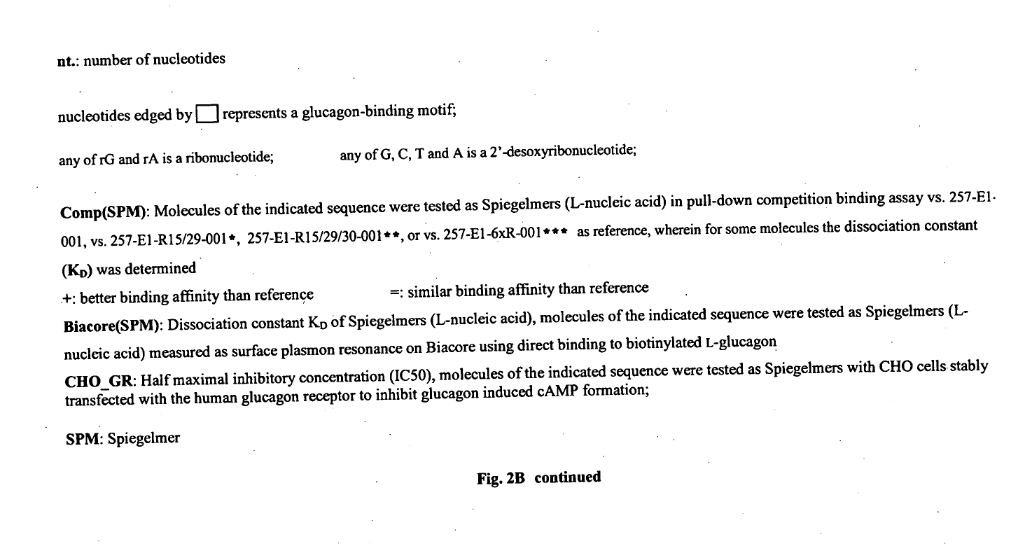

FIGS. 2A-B show derivatives of glucagon binding nucleic acid molecule 257-E1-001, a glucagon binding nucleic acid molecule of “type A”;

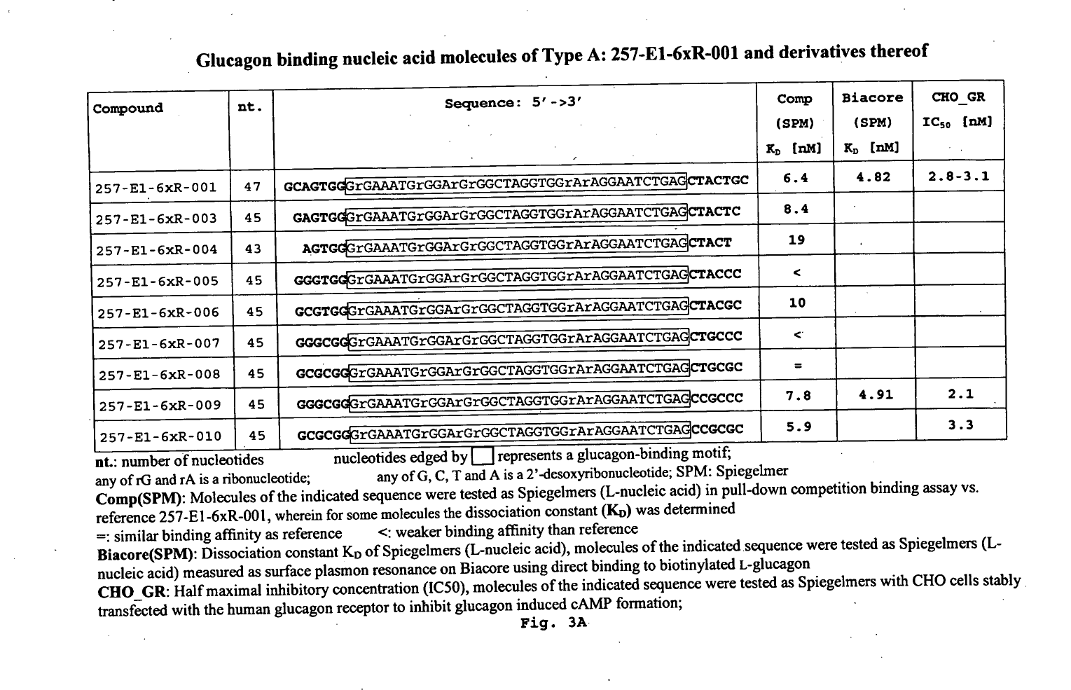

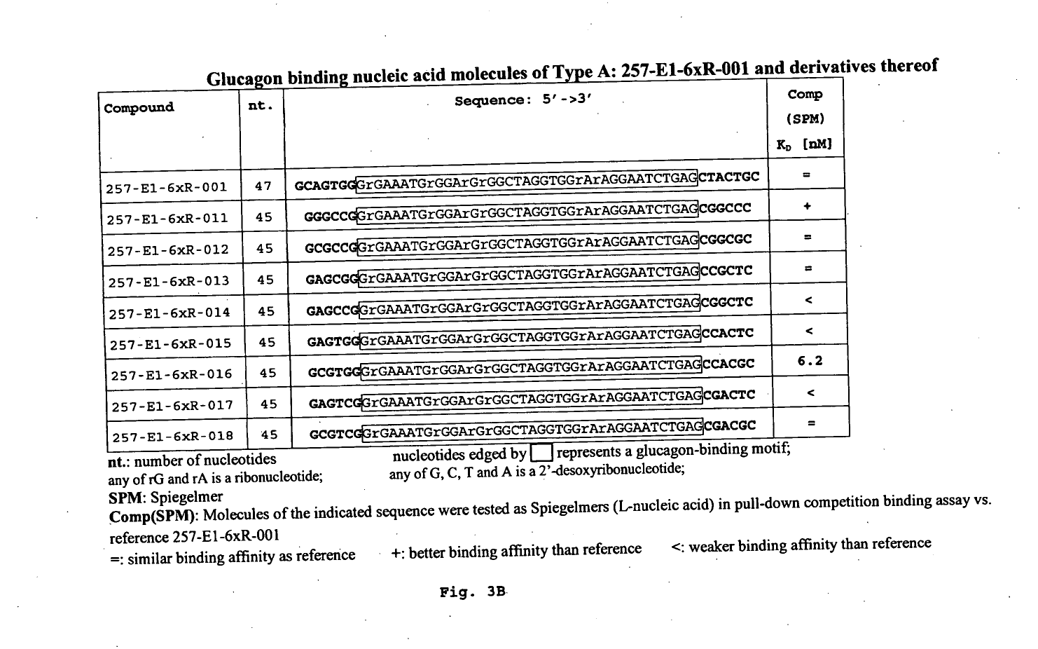

FIGS. 3A-C show derivatives of glucagon binding nucleic acid molecule 257-E1-6xR-001, a glucagon binding nucleic acid molecule of “type A”;

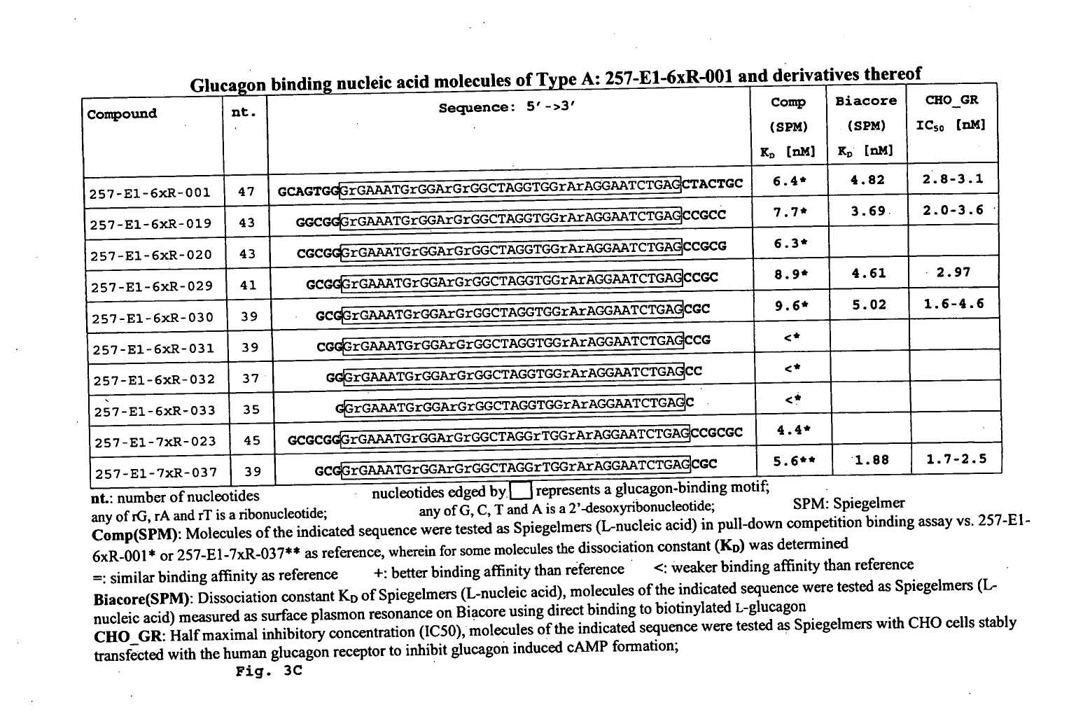

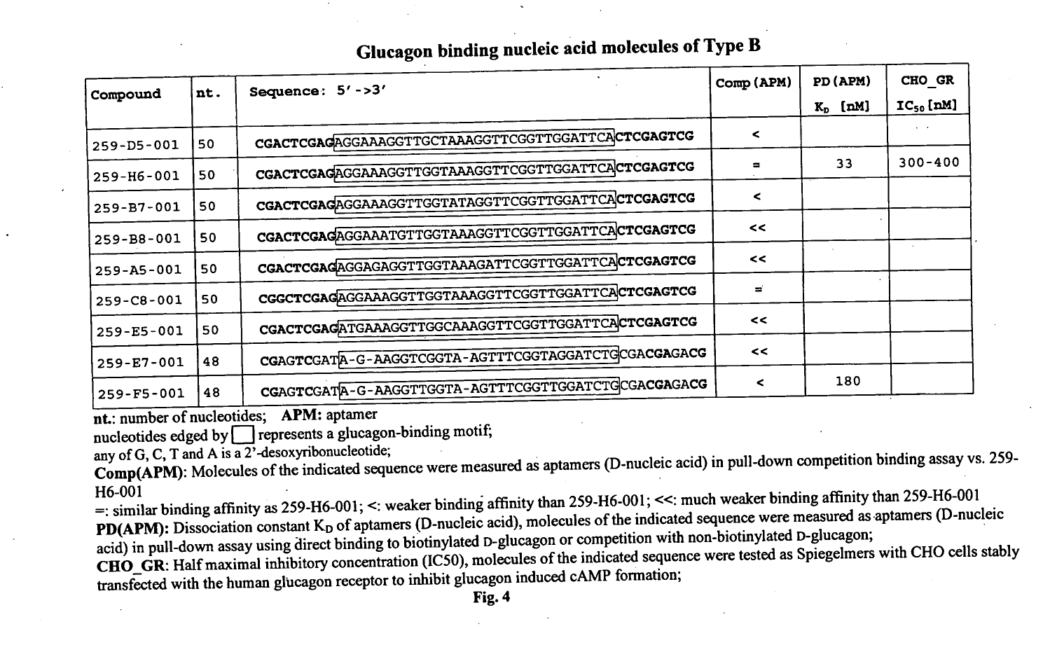

FIG. 4 shows an alignment of sequences of glucagon binding nucleic acid molecules of the invention of “type B”;

FIG. 5 shows derivatives of glucagon binding nucleic acid molecule 259-H6-001, a glucagon binding nucleic acid molecule of “type B”;

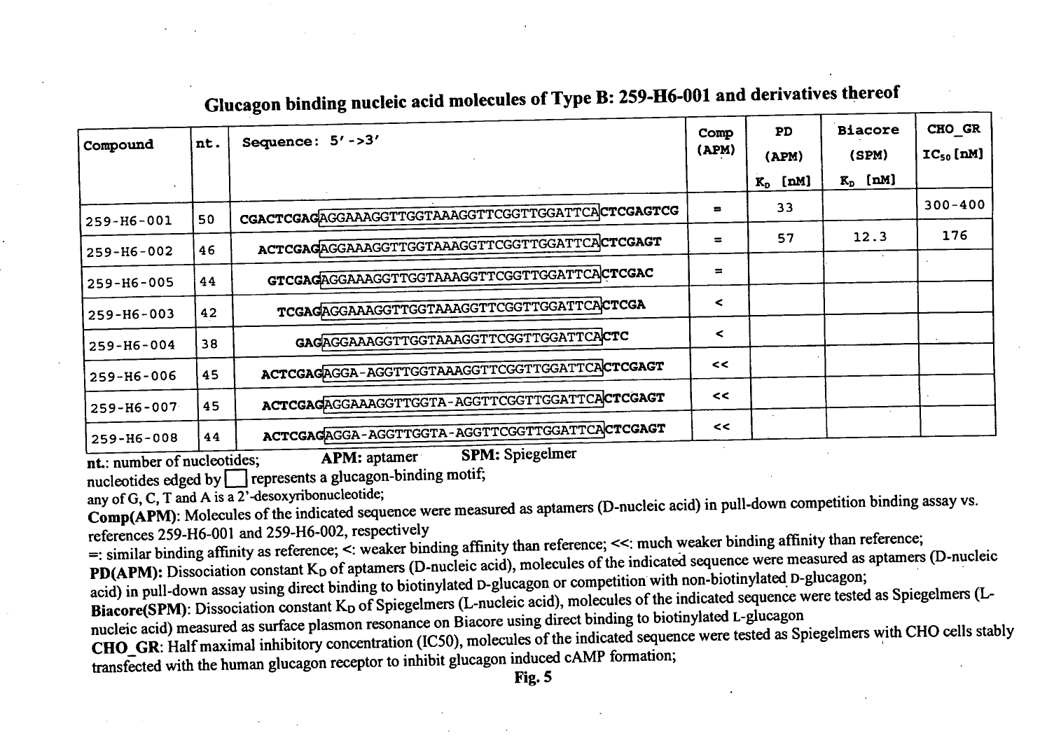

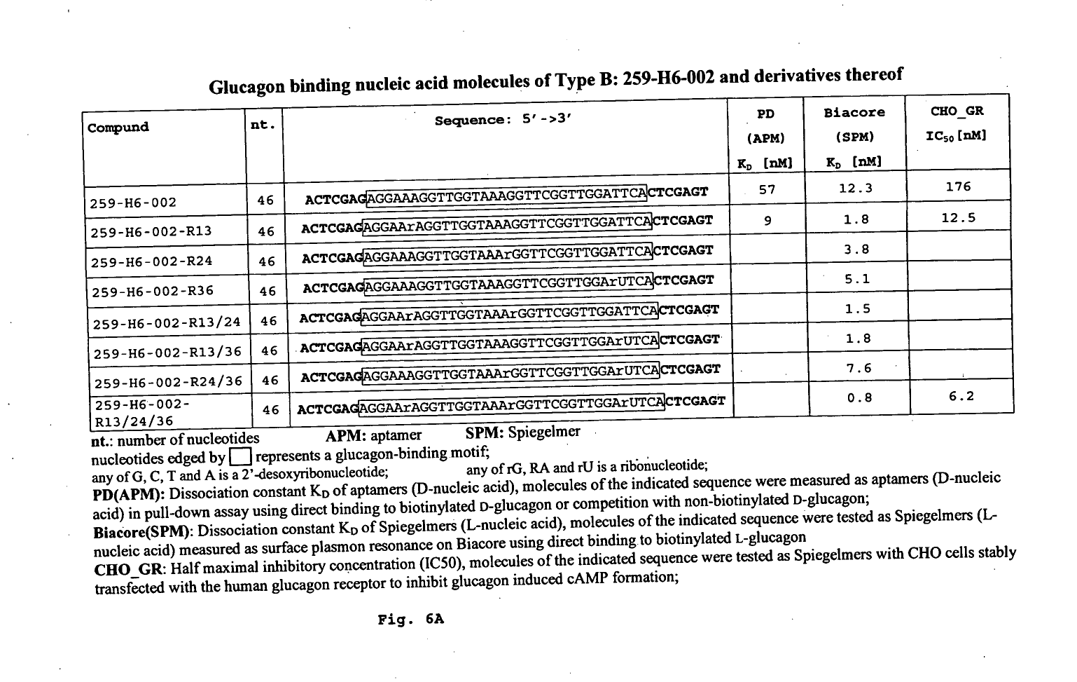

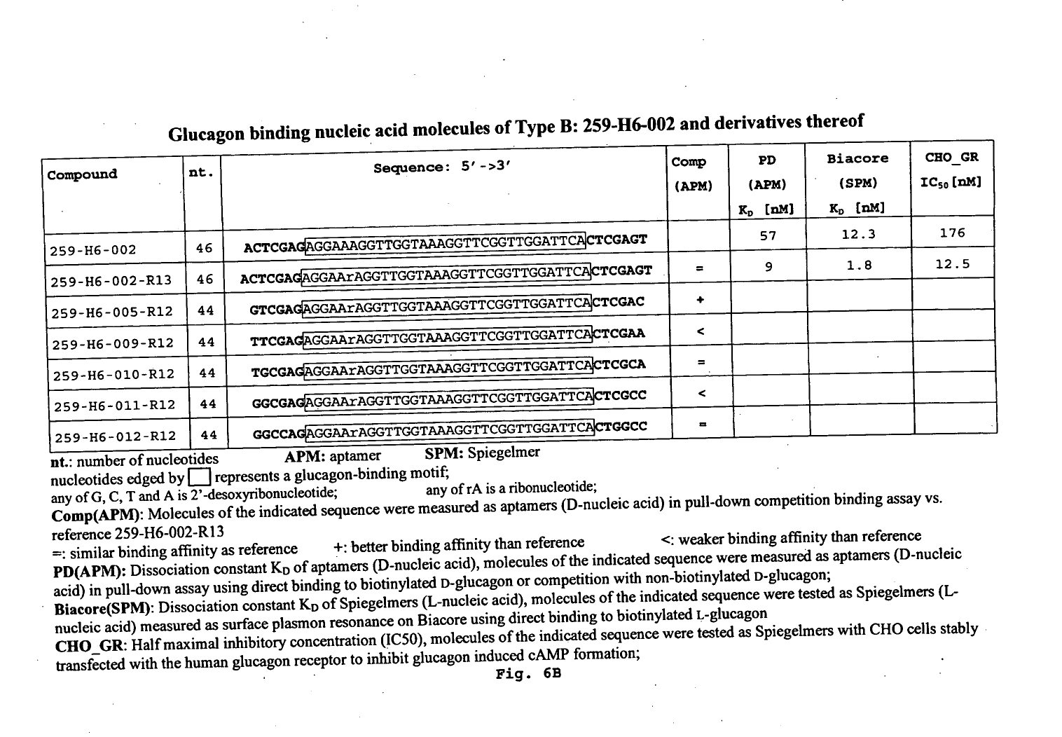

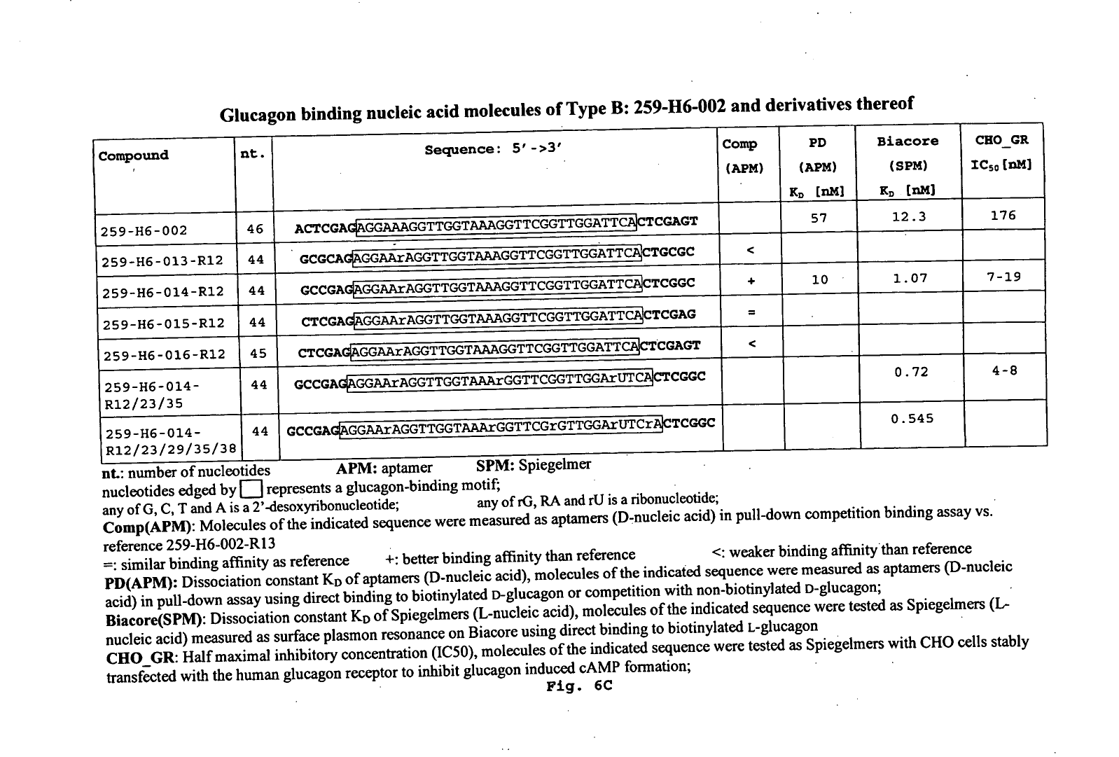

FIGS. 6A-C show derivatives of glucagon binding nucleic acid molecule 259-H6-002, a glucagon binding nucleic acid molecule of “type B”;

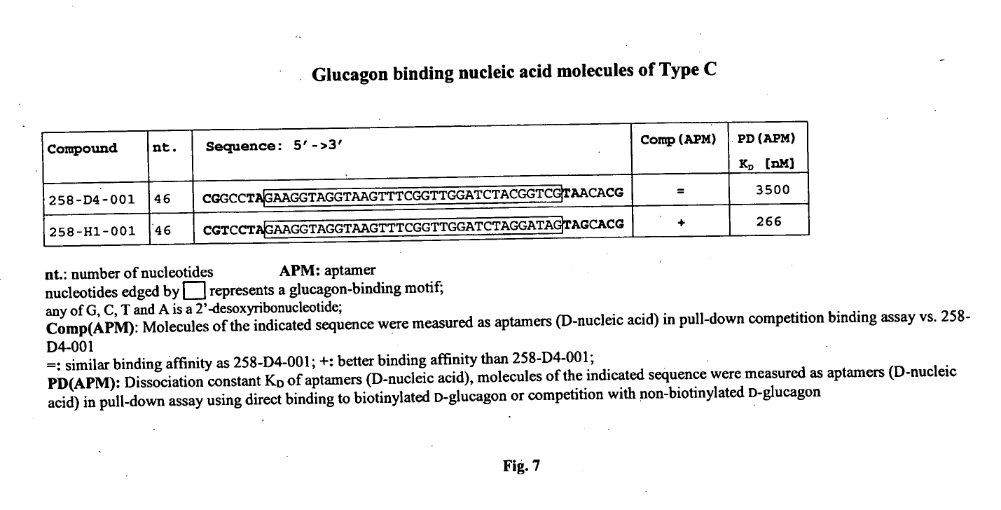

FIG. 7 shows an alignment of sequences of glucagon binding nucleic acid molecules of the invention of “type C”;

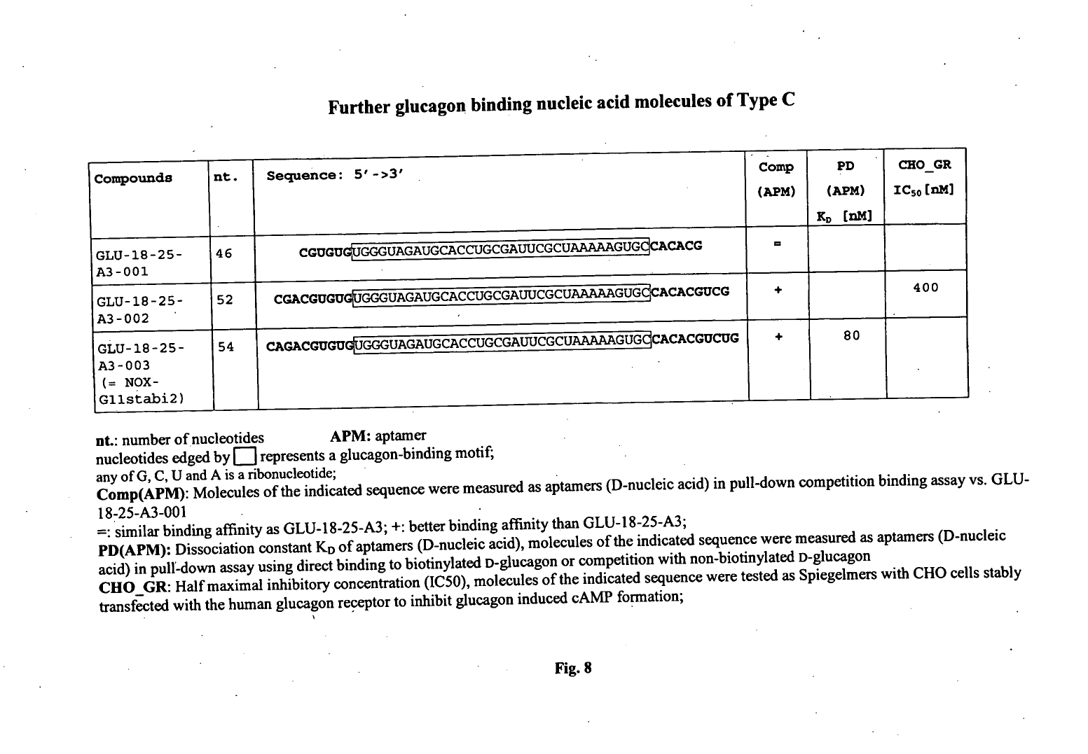

FIG. 8 shows an alignment of sequences of further glucagon binding nucleic acid molecules of the invention of “type C”;

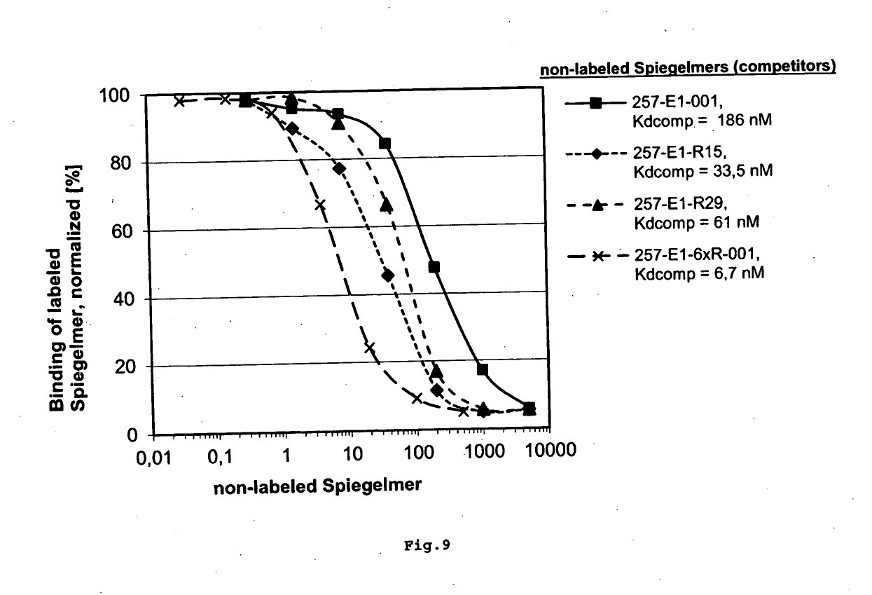

FIG. 9 shows the results of competitive pull-down assays of Spiegelmers 257-E1-001 and its derivatives 257-E1-R15 (also referred to as 257-E1-R15-001), 257-E1-R29 (also referred to as 257-E1-R29-001), and 257-E1-6xR-001 to biotinylated glucagon, whereby Spiegelmer 257-E1-001 or 257-E1-6xR-001 was labeled (→ reference molecule) and the binding of the reference molecule to biotinylated glucagon at 37° C. was competed with 0.032-5000 nM non-labeled Spiegelmers;

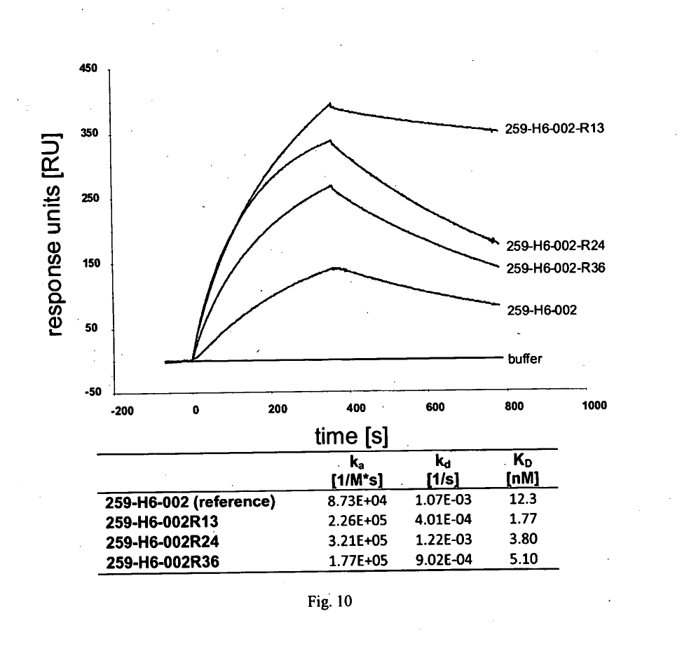

FIG. 10 shows the kinetic evaluation by Biacore measurement of glucagon binding Spiegelmers 259-H6-002-R13, 259-H6-002-R24 and 259-H6-002-R36 vs. Spiegelmer 259-H6-002 to immobilized biotinylated human glucagon, whereby the data for the 500 nM injection of Spiegelmers are shown;

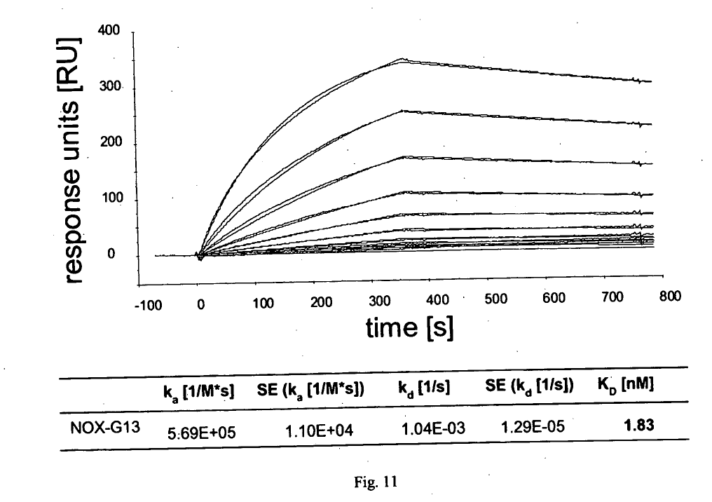

FIG. 11 shows the kinetic evaluation by Biacore measurement of glucagon binding Spiegelmer NOX-G13 to immobilized biotinylated human glucagon, whereby the data for 1000, 500, 250, 125, 62.5, 31.3, 15.6, 7.8, 3.9, and 1.95-0 nM of Spiegelmer NOX-G13 are shown;

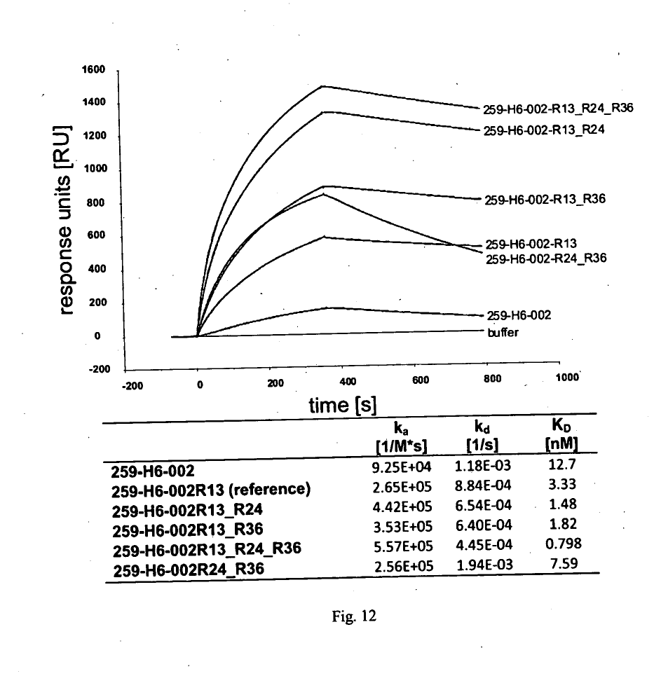

FIG. 12 shows the kinetic evaluation by Biacore measurement of glucagon binding Spiegelmers 259-H6-002-R13, 259-H6-002-R24, 259-H6-002-R36, 259-H6-002-R13-R24, 259-H6-002-R13-R36, 259-H6-002-R24-R36 and 259-H6-002-R13-R24-R36 vs. Spiegelmer 259-H6-002 to immobilized biotinylated human glucagon, whereby the data for the 500 nM injection of Spiegelmers are shown;

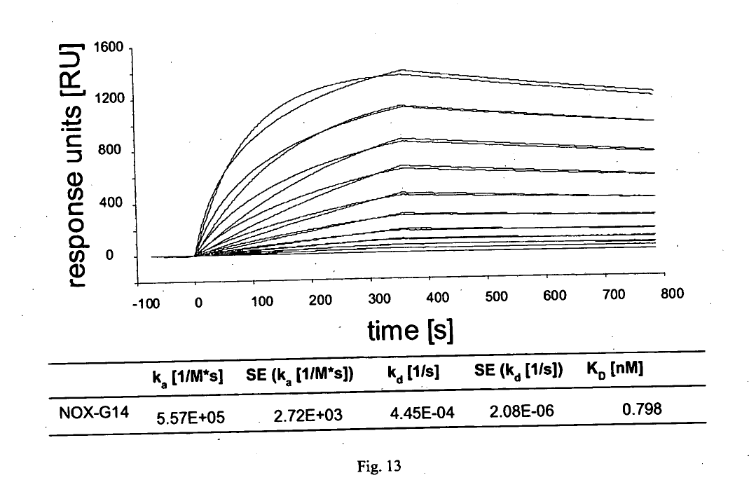

FIG. 13 shows the kinetic evaluation by Biacore measurement of glucagon binding Spiegelmer NOX-G14 to immobilized biotinylated human glucagon, whereby the data for 125, 62.5, 31.3, 15.6, 7.8, 3.9, 1.95 and 0 nM of Spiegelmer NOX-G14 are shown;

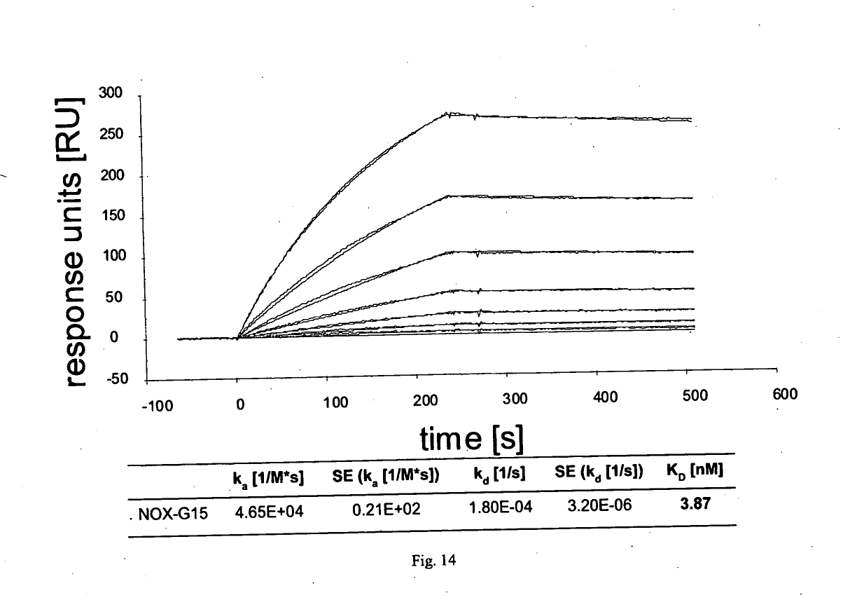

FIG. 14 shows the kinetic evaluation by Biacore measurement of glucagon binding Spiegelmer NOX-G15 to immobilized biotinylated human glucagon, whereby the data for 125, 62.5, 31.3, 15.6, 7.8, 3.9, 1.95 and 0 nM of Spiegelmer NOX-G15 are shown;

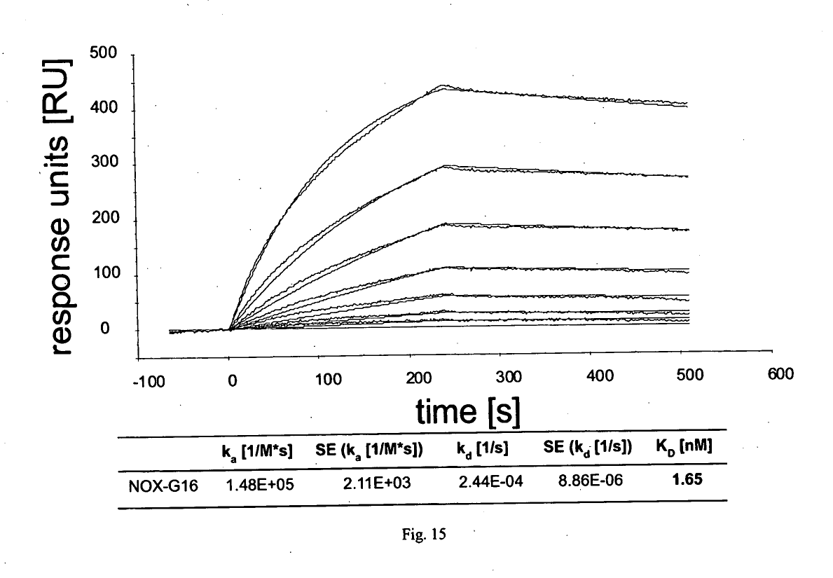

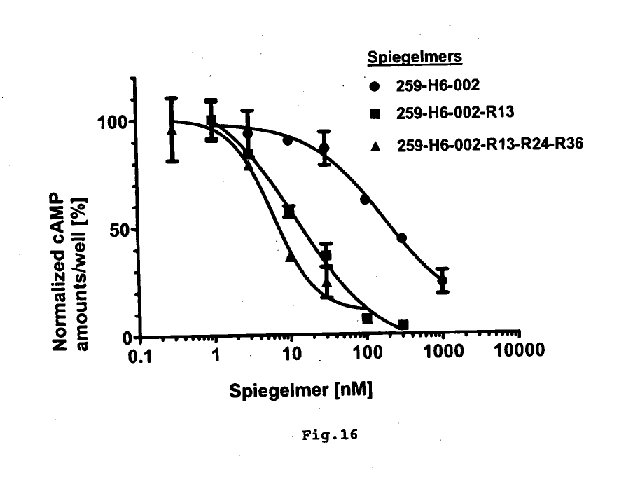

FIG. 15 shows the kinetic evaluation by Biacore measurement of glucagon binding Spiegelmer NOX-G16 to immobilized biotinylated human glucagon, whereby the data for 125, 62.5, 31.3, 15.6, 7.8, 3.9, 1.95 and 0 nM of Spiegelmer NOX-G 16 are shown;

FIG. 16 shows inhibition of glucagon-induced production of cAMP by Spiegelmer 259-H6-002 and its derivatives 259-H6-002-R13 and 259-H6-002-R13-R24-R36 (also referred to as 259-H6-002-R13/24/36), whereby a) the generated amounts of cAMP per well were normalized to the largest value of each data set and depicted as per cent activity against Spiegelmer concentration;

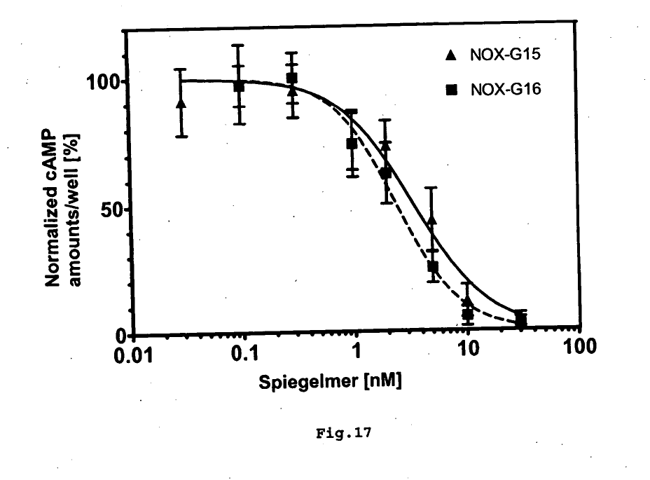

FIG. 17 shows inhibition of glucagon-induced production of cAMP by Spiegelmers NOX-G15 and NOX-G16, whereby a) the generated amounts of cAMP per well were normalized to the largest value of each data set and depicted as per cent activity against Spiegelmer concentration, b) the Spiegelmer concentrations at which cAMP production is inhibited by 50% (IC₅₀) were calculated using nonlinear regression (four parameter fit) with Prism5 software, c) the IC₅₀ values for NOX-G15 (5 independent experiments) and NOX-G16 (3 independent experiments) determined were 3.44 nM and 2.43 nM, respectively;

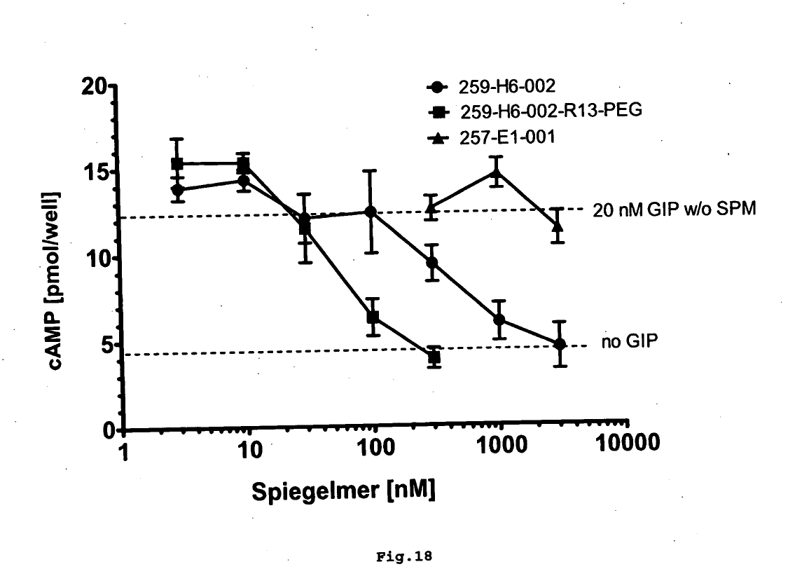

FIG. 18 shows inhibition of GIP-induced production of cAMP by Spiegelmers 259-H6-002, 259-H6-002-R13-PEG (also referred to as NOX-G13) and 257-E1-001, whereby a) the generated amounts of cAMP per well were normalized to the largest value of each data set and depicted as per cent activity against Spiegelmer concentration, b) the Spiegelmer concentrations at which cAMP production is inhibited by 50% (IC₅₀) were calculated using nonlinear regression (four parameter fit) with Prism5 software, and c) Spiegelmers 259-H6-002 and 259-H6-002-R13-PEG showed dose-dependent inhibition of GIP-induced cAMP generation and Spiegelmer 257-E1-001 did not show inhibitory activity against GIP;

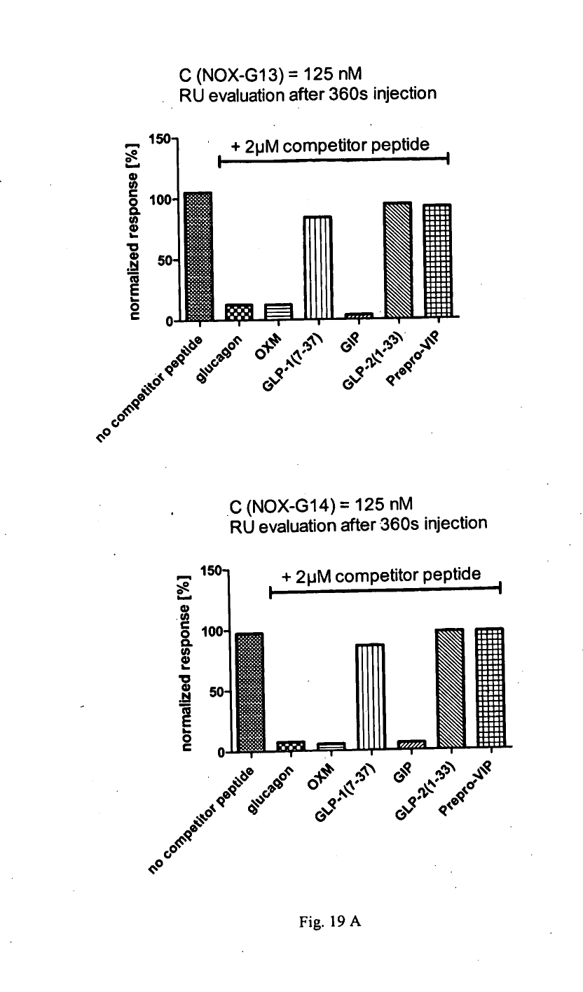

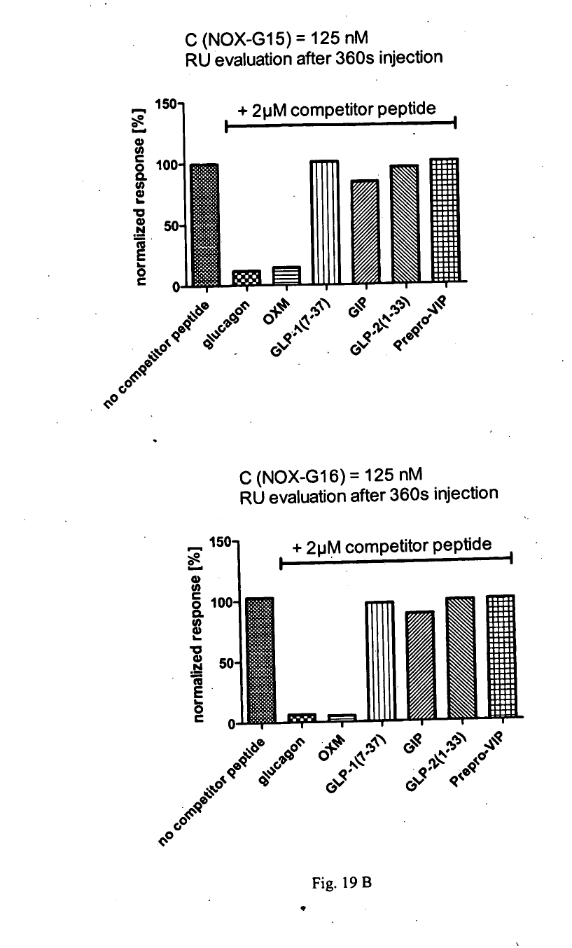

FIG. 19 shows data of competitive Biacore selectivity assays with Spiegelmers NOX-G13, NOX-G14, NOX-G15 and NOX-G16 and the competitor peptides glucagon, Glucagon-dependent insulinotropic polypeptide (abbr. GIP), Glucagon-like peptide-1 (abbr. GLP-1) (7-37), Glucagon-like peptide-2 (abbr. GLP-2) (1-33), Oxyntomodulin (abbr. OXM and Vasoactive intestinal peptide (abbr. VIP); control means “no competitor peptide”; data were normalized to the control (100%);

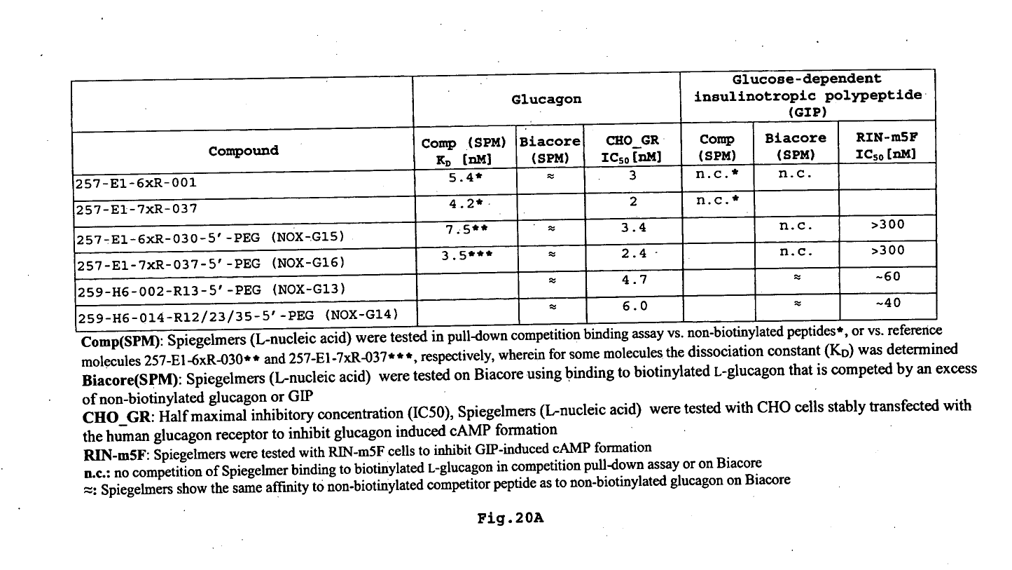

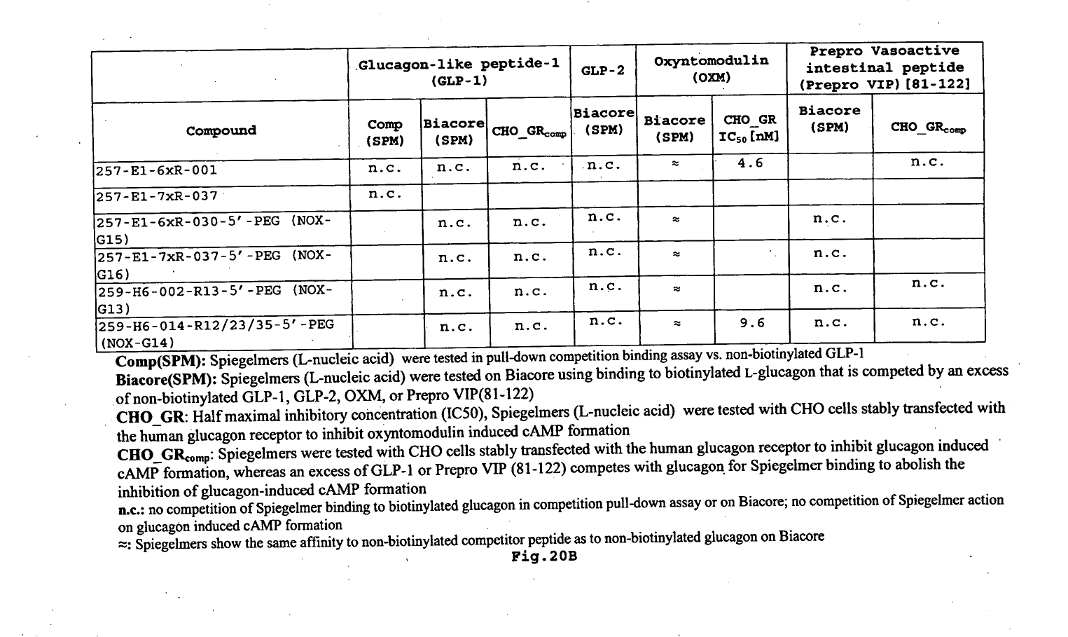

FIG. 20A-B show data regarding the binding of Spiegelmers 257-E1-6xR-001, 257-E1-7xR-037, 257-E1-6xR-030-5′-PEG (also referred to as NOX-G15), 257-E1-7xR-037-5′-PEG (also referred to as NOX-G16), 259-H6-002-R13-5′-PEG (also referred to as NOX-G13) and 259-H6-014-R12/23/35-5′-PEG (also referred to as NOX-G14) to glucagon, GIP, GLP-1, OXM, and VIP as well as the competition of GIP, GLP-1, OXM, and VIP with said the Spiegelmers' effect on the glucagon induced cAMP generation in vitro;

FIG. 21 shows the amino acid sequences of Glicentin, Glicentin-related polypeptide (short name=GRPP), Oxyntomodulin (short name=OXY, short name=OXM), Glucagon, Glucagon-like peptide 1 (short name=GLP-1), Glucagon-like peptide 1(7-37) (short name=GLP-1(7-37)), Glucagon-like peptide 1(7-36) (short name=GLP-1(7-36)) and Glucagon-like peptide 2 (short name=GLP-2);

FIG. 22 shows the amino acid sequences of glucagon of different species;

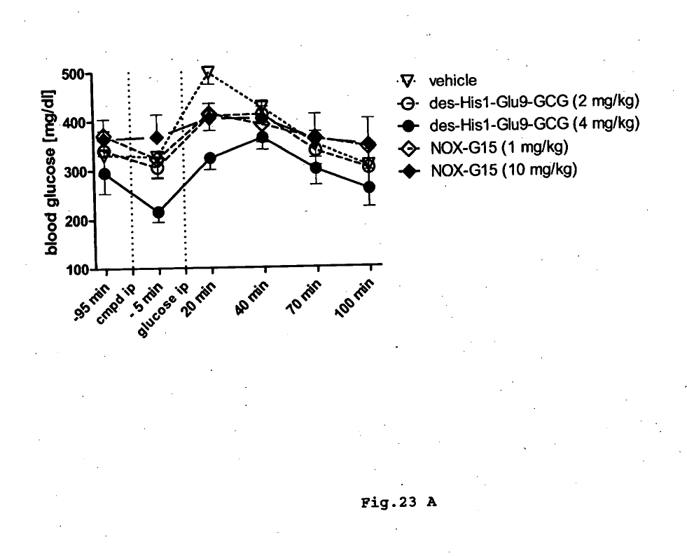

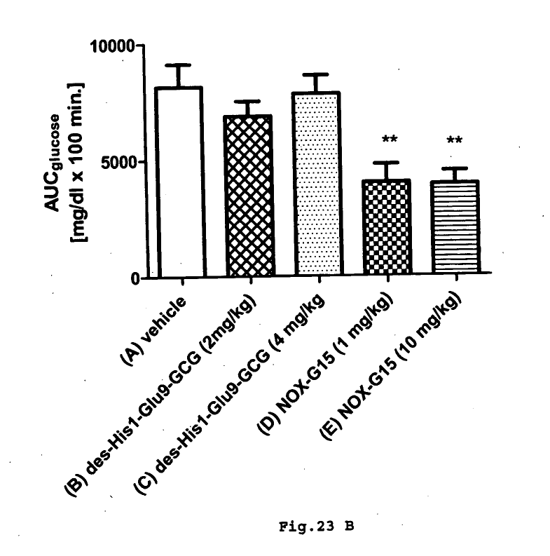

FIG. 23A-B show the results of an intraperitoneal glucose tolerance test in the type 1 diabetes mellitus mouse model with:

FIG. 23A indicating blood glucose over time (mean and SEM); and

FIG. 23B indicating Area under the curve (AUC) determination;
data were analyzed using One Way ANOVA and Tukey posttest; significance levels versus vehicle group: * means p<00.5, ** means p<0.01;

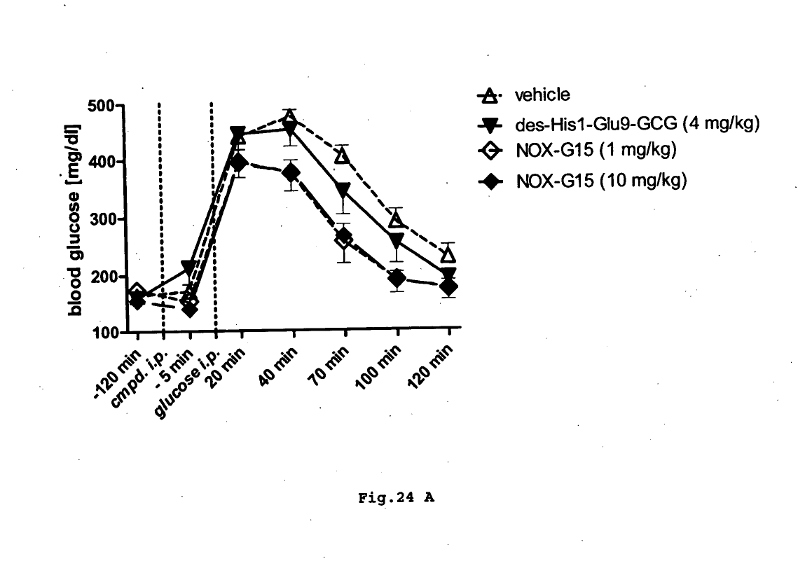

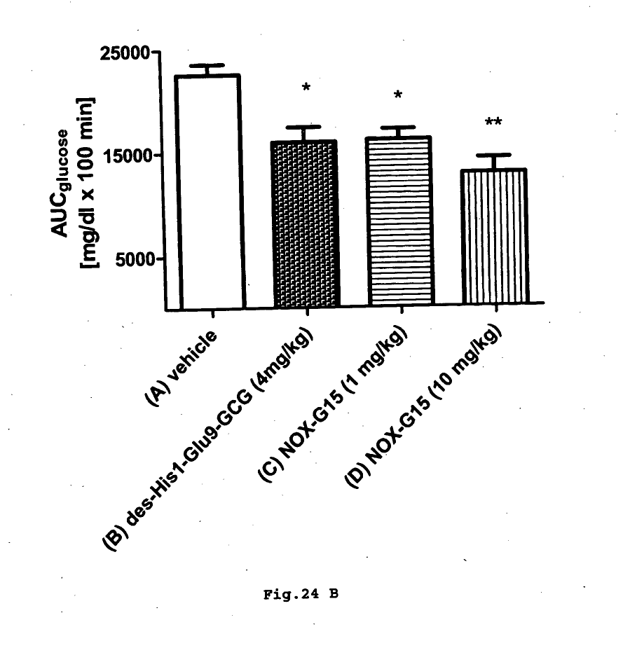

FIG. 24A-B show intraperitoneal glucose tolerance test in the type 2 diabetes mellitus mouse model:
(A): indicating blood glucose over time; and
(B): indication Area under the curve (AUC) determination;
data were analyzed using One Way ANOVA and Tukey posttest;
significance levels versus vehicle group: * p<0.05, ** p<0.01;

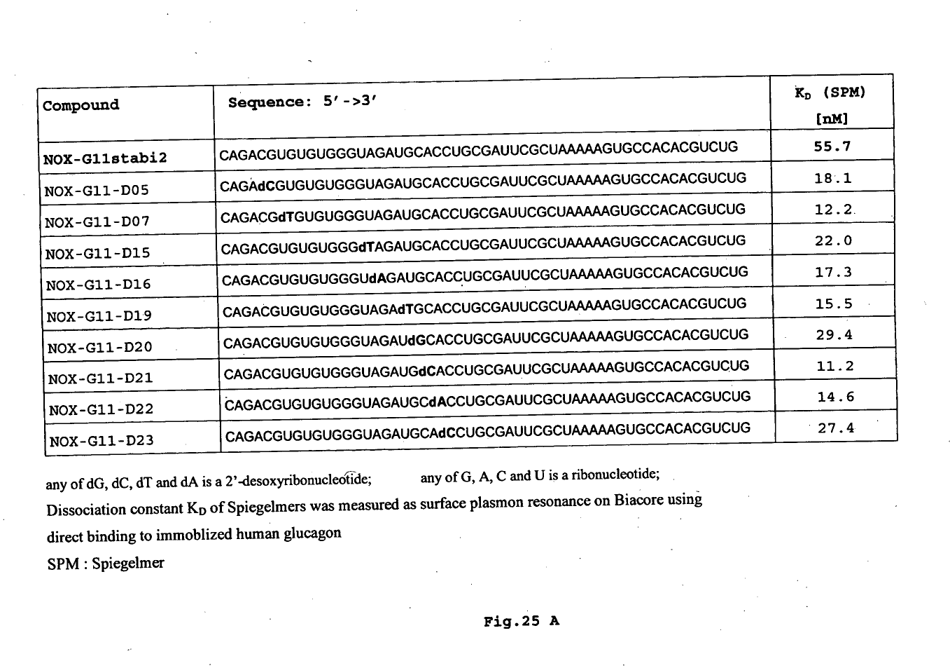

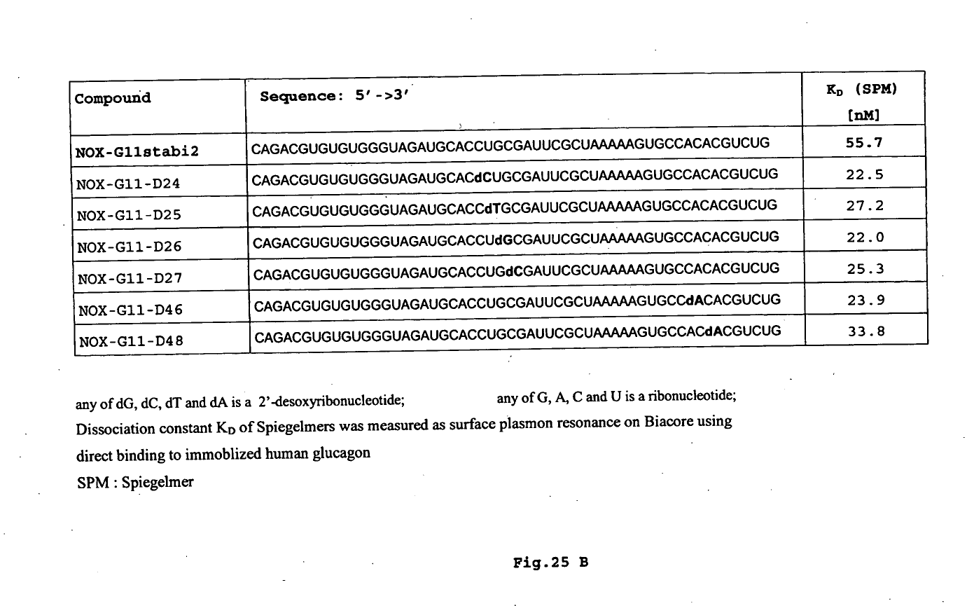

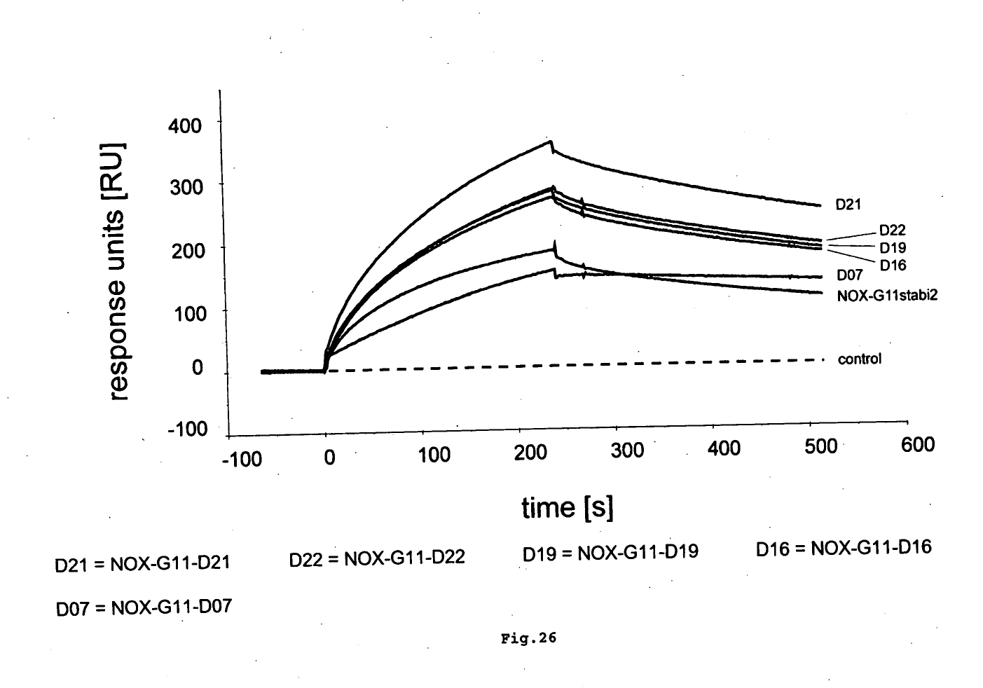

FIGS. 25A-B shows derivatives of glucagon binding nucleic acid molecule NOX-G11stabi, a glucagon binding nucleic acid molecule of the invention of “type C”;

FIG. 26 shows the kinetic evaluation by Biacore measurement of glucagon binding Spiegelmers NOX-G 11stabi2, NOX-G 11-D07, NOX-G11-D16, NOX-G 1-D19, NOX-G11-D21 and NOX-G11-D22 to immobilized biotinylated human glucagon;

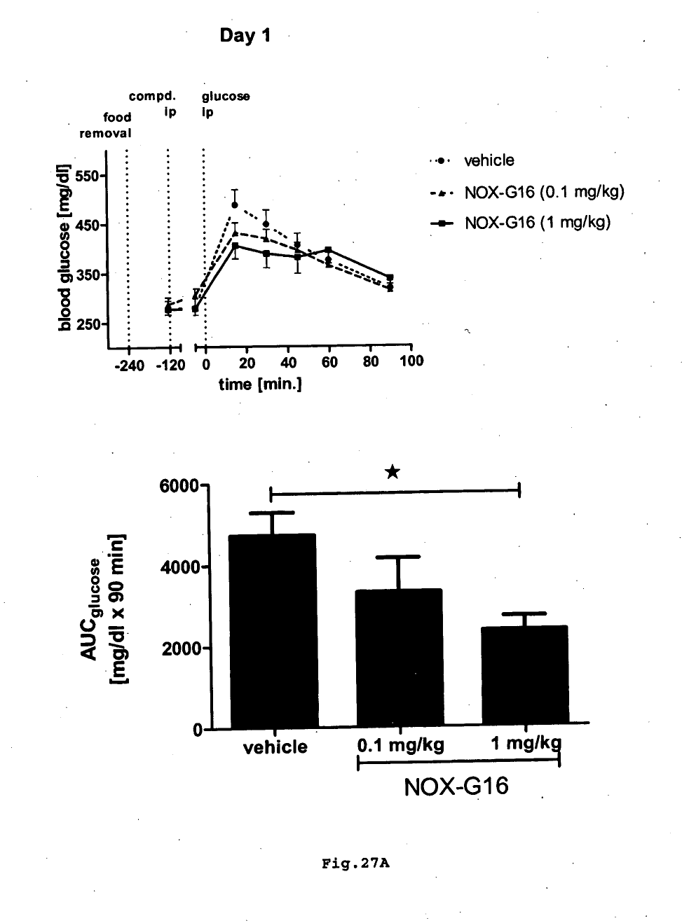

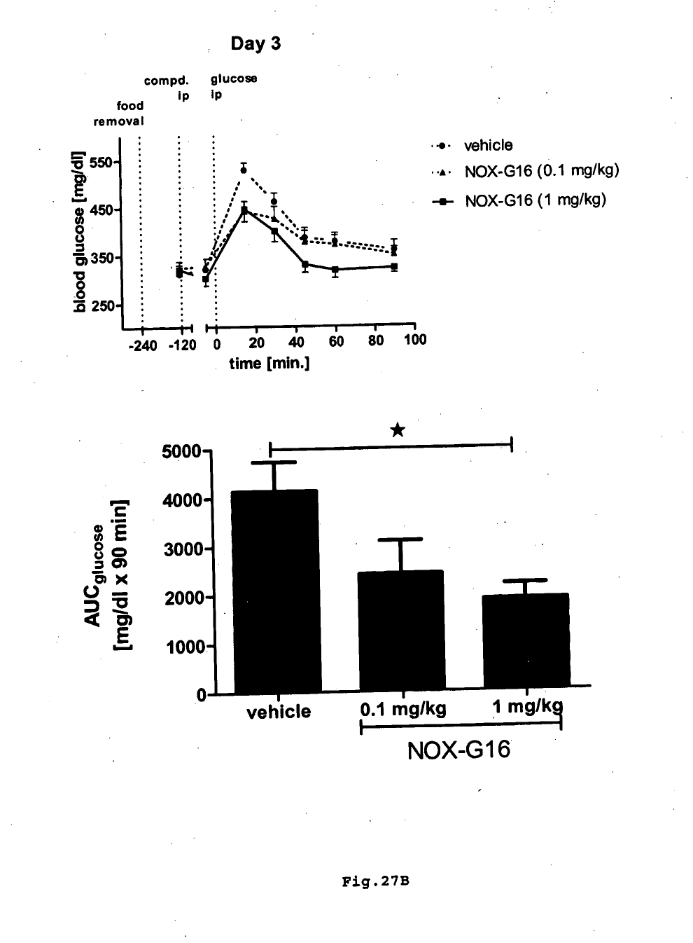

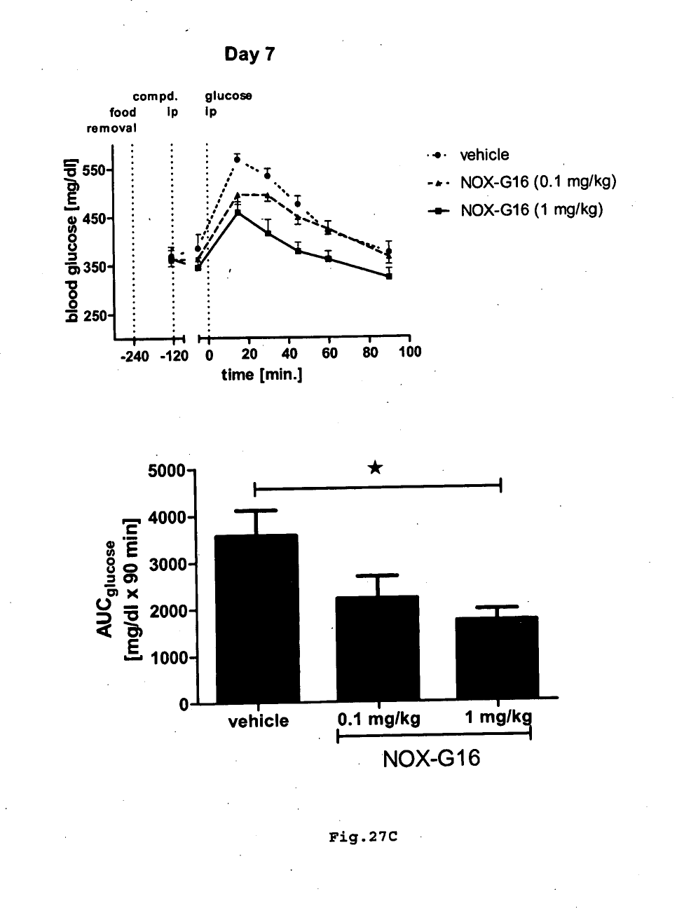

FIG. 27 shows the intraperitoneal glucose tolerance test in the type 1 diabetes mellitus mouse model,
(A): on day 1 after a single dose of NOX-G16; (B) on day 5 after five doses (q1d) of NOX-G16 and (C) on day 7 after seven doses (q1d) of NOX-G 16:
upper panel: blood glucose over time. (mean and SEM);
lower panel: Area under the curve (AUC) determination;
data were analyzed using One Way ANOVA and Tukey posttest;
significance levels versus vehicle group: * p<00.5;

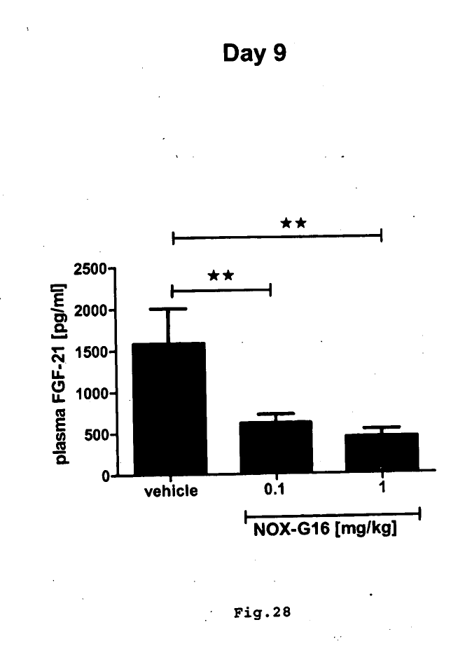

FIG. 28 shows the plasma FGF-21 levels on day 9 after nine NOX-G16 doses (q1d). Data were analyzed using One Way ANOVA and Tukey posttest; significance levels versus vehicle group: * p<00.5, **p<0.01;

FIG. 29 shows the 2′deoxyribonucleotides that the nucleic acid molecules according to the present invention consist of; and

FIG. 30 A-B shows the ribonucleotides that the nucleic acid molecules according to the present invention consist of.

### EXAMPLE 1

### Nucleic Acid Molecules that Bind Glucagon

Several glucagon binding nucleic acid molecules and derivatives thereof were identified: the nucleotide sequences of which are depicted in FIGS. 1 to 8. The glucagon binding nucleic acid molecules were characterized as
a) aptamers, i. e. as D-nucleic acid molecules using a direct pull-down assay (Example 3) and/or a comparative competition pull-down assay (Example 3);
b) spiegelmers, i. e. L-nucleic acid using a comparative competition pull-down assay (Example 3), by surface plasmon resonance measurement (Example 4), and by an in vitro assay with the human glucagon receptor (Example 5). Moreover spiegelmers were tested in vivo (Example 8).

The spiegelmers and aptamers were synthesized as described in Example 2.

The nucleic acid molecules thus generated exhibit slightly different sequences, whereby three main types were identified and defined as glucagon binding nucleic acid molecules: glucagon binding nucleic acid molecules of Type A (FIGS. 1 to 3), glucagon binding nucleic acid molecules of Type B (FIGS. 4 to 6) and glucagon binding nucleic acid molecules of Type C (FIGS. 7 and 8).

For definition of 2′-deoxynucleotide sequence motifs, the IUPAC abbreviations for ambiguous nucleotides are used:

| | | |
|---|---|---|
| S | strong | G or C; |
| W | weak | A or T; |
| R | purine | G or A; |
| Y | pyrimidine | C or T; |
| K | keto | G or T; |
| M | imino | A or C; |
| B | not A | C or T or G; |
| D | not C | A or G or T; |
| H | not G | A or C or T; |
| V | not T | A or C or G; |
| N | all | A or G or C or T |

If not indicated to the contrary, any nucleic acid sequence or sequence of stretches, respectively, is indicated in the 5′→3′ direction.

### 1.1 Glucagon Binding Nucleic Acid Molecules of Type A

As depicted in FIG. 1 to FIG. 3 glucagon binding nucleic acid molecules of Type A comprise one central stretch of nucleotides defining a potential glucagon binding motif.

In general, glucagon binding nucleic acid molecules of Type A comprise at the 5′-end and the 3′-end terminal stretches of nucleotides: the first terminal stretch of nucleotides and the second terminal stretch of nucleotides. The first terminal stretch of nucleotides and the second terminal stretch of nucleotides can hybridize to each other, whereby upon hybridization a double-stranded structure is formed. However, such hybridization is not necessarily given in the molecule.

The three stretches of nucleotides of glucagon binding nucleic acid molecules of Type A—a first terminal stretch of nucleotides, a central stretch of nucleotides and a second terminal stretch of nucleotides—are arranged in 5′→3′-direction as follows: the first terminal stretch of nucleotides—the central stretch of nucleotides—the second terminal stretch of nucleotides. Alternatively, however, the first terminal stretch of nucleotides, the central stretch of nucleotides and the second terminal stretch of nucleotides are arranged to each other in 5′→3′-direction as follows: the second terminal stretch of nucleotides—the central stretch of nucleotides—the first terminal stretch of nucleotides.

The sequences of the defined stretches may be different between the glucagon binding nucleic acid molecules of Type A which influences the binding affinity to glucagon. Based on binding analysis of the different glucagon binding nucleic acid molecules of Type A the central stretch of nucleotides and their nucleotide sequences as described in the following are individually and more preferably in their entirety essential for binding to human glucagon.

The glucagon binding nucleic acid molecules of Type A according to the present invention are shown in FIGS. 1 to 3. All of them were tested as aptamers and/or spiegelmers for their ability to bind glucagon. The first glucagon binding nucleic acid molecule of Type A that was characterized for its binding affinity to glucagon was nucleic acid molecule 257-E1-001 that consists of 2′-deoxyribonucleotides. The equilibrium binding constant K_{D} of nucleic acid molecule 257-E1-001 was determined as aptamer and as spiegelmer by direct pull-down binding assays (K_{D}_{_{—}}ₐₚₜₐₘₑᵣ=137 nM, K_{D}_{_{—}}_{spiegelmer}=179 nM; FIG. 1).

The glucagon binding nucleic acid molecules 257-A1-001, 257-D4-001, 257-F4-001, 257-B3-001, 257-D3-001, 257-E4-001, 257-C4-001, 257-C1-001 and 257-H2-001—all of them consisting of 2′-deoxyribonucleotides—were tested as aptamers in comparative competition pull-down assays vs. glucagon binding nucleic acid 257-E1-001. Glucagon binding nucleic acid molecule 257-E4-001 showed similar binding affinity as 257-E1-001. Glucagon binding nucleic acid molecules 257-A1-001, 257-F4-001, 257-C1-001 and 257-H2-001 showed weaker binding affinity in comparison to glucagon binding nucleic acid molecule 257-E1-001. Glucagon binding nucleic acid molecules 257-D4-001, 257-B3-001, 257-D3-001 and 257-C4-001 showed much weaker binding affinity in comparison to glucagon binding nucleic acid molecule 257-E1-001 (FIG. 1).

Derivatives 257-E1-002, 257-E1-003, 257-E1-004 and 257-E1-005 of glucagon binding nucleic molecule 257-E1-001 comprise a first and a second terminal stretch of nucleotides each with six, five or four nucleotides whereby glucagon binding nucleic molecule 257-E1-001 comprises a first and second terminal stretch of nucleotides each with seven nucleotides, respectively. Derivatives 257-E1-002, 257-E1-003, 257-E1-004 and 257-E1-005 of glucagon binding nucleic molecule 257-E1-001 showed reduced binding affinity in a comparative competition pull-down assay compared to glucagon binding nucleic molecule 257-E1-001 (FIG. 2A). Accordingly, truncation of the first and the second terminal stretch of nucleotides of glucagon binding nucleic acid molecule 257-E1-001 led to reduced binding affinity to glucagon.

Glucagon binding nucleic acid molecules 257-A1-001, 257-D4-001, 257-F4-001, 257-B3-001, 257-D3-001, 257-E4-001, 257-C4-001, 257-C1-001, 257-H2-001, 257-E1-001 and its derivatives 257-E1-002, 257-E1-003, 257-E1-004 and 257-E1-005 share the sequence

[SEQ ID NO: 192]
5′ BGAAATGGGAGGGCTAKGYGGAAGGAATCTRRR 3′

for the central stretch of nucleotides, whereby G, A, T, C, B, Y, K, and R are 2′-deoxyribonucleotides, wherein
a) in a preferred embodiment the central stretch of nucleotides comprises the sequence

[SEQ ID NO: 193]
5′ TGAAATGGGAGGGCTAGGTGGAAGGAATCTGAG 3′,

wherein G, A, T and C are 2′-deoxyribonucleotides;
b) in a preferred embodiment the central stretch of nucleotides comprises the sequence

[SEQ ID NO: 194]
5′ TGAAATGGGAGGGCTAGGTGGAAGGAATCTGAA 3′,

wherein G, A, T and C are 2′-deoxyribonucleotides;
c) in a preferred embodiment the central stretch of nucleotides comprises the sequence

[SEQ ID NO: 195]
5′ CGAAATGGGAGGGCTAGGTGGAAGGAATCTGAG 3′,

wherein G, A, T and C are 2′-deoxyribonucleotides;
d) in a preferred embodiment the central stretch of nucleotides comprises the sequence

[SEQ ID NO: 196]
5′ GGAAATGGGAGGGCTAGGTGGAAGGAATCTGAG 3′,

wherein G, A, T and C are 2′-deoxyribonucleotides.

Glucagon binding nucleic acid molecules 257-E4-001 and 257-E1-001 showed the best binding affinity to glucagon and comprise the following sequences for the central stretch:

### a) 257-E4-001:

[SEQ ID NO: 195]
5′ CGAAATGGGAGGGCTAGGTGGAAGGAATCTGAG 3′

b) 257-E1-001 and its derivatives:

[SEQ ID NO: 196]
5′ GGAAATGGGAGGGCTAGGTGGAAGGAATCTGAG 3′,

whereby G, A, T, C are 2′-deoxyribonucleotides.

The inventors surprisingly showed in a comparative competition spiegelmer pull-down assay format that the binding affinity of glucagon binding nucleic acid molecule 257-E1-001 was improved by replacing 2′-deoxyribonucleotides by ribonucleotides within the sequence of the central stretch of nucleotides. The 2′-deoxyribonucleotides and ribonucleotides are shown in FIGS. 29 and 30A-B, wherein in Example 1.1 and in the corresponding figures the following abbreviations were used: G is 2′deoxy-guanosine (5′monophosphate), C is 2′deoxy-cytidine (5′monophosphate), A is 2′deoxy-adenosine (5′monophosphate), T is 2′deoxy-thymidine (5′monophosphate), rG is guanosine (5′monophosphate), rT is thymidine (5′monophosphate) and rA is adenosine (5′monophosphate). In particular replacing up to seven 2′-deoxyribonucleotides by ribonucleotides in the glucagon binding nucleic acid molecule 257-E1-001 resulted in improved binding affinity to glucagon by a factor of up to more than forty.

In more detail, the inventors have surprisingly found that
a) replacing one 2′-deoxyribonucleotide by one ribonucleotide at position 2, 8, 11, 12, 22 or 23 in the central stretch of nucleotides of glucagon binding nucleic acid molecule 257-E1-001 resulted. in improved binding affinity to biotinylated glucagon in comparison to the binding affinity of glucagon binding nucleic acid molecule 257-E1-001 (see FIGS. 2B and 9; spiegelmers 257-E1-R09-001, 257-E1-R15-001, 257-E1-R18-001, 257-E1-R19-001, 257-E1-R29-001, 257-E1-R30-001);
b) replacing two 2′-deoxyribonucleotides by two ribonucleotides at positions 8 and 22 or 22 and 23 in the central stretch of nucleotides of glucagon binding nucleic acid molecule 257-E1-001 resulted in improved binding affinity to biotinylated glucagon in comparison to the binding affinity of glucagon binding nucleic acid molecule 257-E1-001 (see FIG. 2B; spiegelmers 257-E1-R15/29-001, 257-E1-R29/30-001);
c) replacing three 2′-deoxyribonucleotides by three ribonucleotides at positions 8, 22 and 23 or 11, 22 and 23 in the central stretch of nucleotides of glucagon binding nucleic acid molecule 257-E1-001 resulted in improved binding affinity to biotinylated glucagon in comparison to the binding affinity of glucagon binding nucleic acid molecule 257-E1-001 (see FIG. 2B; spiegelmers 257-E1-R15/29/30-001, 257-E1-R18/29/30-001);
d) replacing four 2′-deoxyribonucleotides by four ribonucleotides at positions 8, 11, 22 and 23 in the central stretch of nucleotides of glucagon binding nucleic acid molecule 257-E1-001 resulted in improved binding affinity to biotinylated glucagon in comparison to the binding affinity of glucagon binding nucleic acid molecule 257-E1-001 (see FIG. 2B; spiegelmer 257-E1-R15/18/29/30-001);
e) replacing six 2′-deoxyribonucleotides by six ribonucleotides at positions 2, 8, 11, 12, 22 and 23 in the central stretch of nucleotides of glucagon binding nucleic acid molecule 257-E1-001 resulted in improved binding affinity to biotinylated glucagon in comparison to the binding affinity of glucagon binding nucleic acid molecule 257-E1-001 (see FIGS. 2B and 9; spiegelmer 257-E1-6xR-001); and
f) replacing seven 2′-deoxyribonucleotides by seven ribonucleotides at positions 2, 8, 11, 12, 19, 22 and 23 in the central stretch of nucleotides of glucagon binding nucleic acid molecule 257-E1-001 resulted in improved binding affinity to biotinylated glucagon in comparison to the binding affinity of glucagon binding nucleic acid molecule 257-E1-001 (see FIG. 3C; spiegelmers 257-E1-7xR-023 and 257-E1-7xR-037).

Based on the data shown that replacing 2′-deoxyribonucleotides by ribonucleotides at several positions of the central stretch of nucleotides of glucagon binding nucleic acid molecules of Type A led to improved binding to glucagon, the central stretch of all tested glucagon binding nucleic acid molecules of Type A can be summarized in the following generic formula

[SEQ ID NO: 173]
5′ Bn₁AAATGn₂GAn₃n₄GCTAKGX₅GGn₆n₇GGAATCTRRR 3′,

wherein n₁ is G or rG, n₂ is G or rG, n₃ is G or rG, n₄ is G or rG, n₅ is Y or rT, n₆ is A or rA, n₇ is A or rA, and wherein G, A, T, C, B, K, Y and R are 2′-deoxyribonucleotides, and rG, rA and rT are ribonucleotides.

Glucagon binding nucleic acid molecules 257-A1-001, 257-F4-001, 257-E4-001, 257-C1-001, 257-H2-001, 257-E1-001 and the derivatives of 257-E1-001 comprising ribonucleotides instead of 2′-deoxyribonucleotides at several positions of the central stretch of nucleotides showed better binding affinity to glucagon than other glucagon binding nucleic acid molecules of Type A and share the following sequences for the central stretch:

[SEQ ID NO: 174]
5′ Bn₁AAATGn₂GAn₃n₄GCTAGGn₅GGn₆n₇GGAATCTGAR 3′,

wherein n₁ is G or rG, n₂ is G or rG, n₃ is G or rG, n₄ is G or rG, n₅ is T or rT, n₆ is A or rA, n₇ is A or rA, and wherein G, A, T, C, B, and R are 2′-deoxyribonucleotides, and rG, rA and rT are ribonucleotides, wherein
a) in a preferred embodiment the central stretch of nucleotides comprises the sequence

[SEQ ID NO: 175]
5′ Tn₁AAATGn₂GAn₃n₄GCTAGGn₅GGn₆n₇GGAATCTGAG 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides;
b) in a preferred embodiment the central stretch of nucleotides comprises the sequence

[SEQ ID NO: 176]
5′ Tn₁AAATGn₂GAn₃n₄GCTAGGn₅GGn₆n₇GGAATCTGAA 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides;
c) in a preferred embodiment the central stretch of nucleotides comprises the sequence

[SEQ ID NO: 177]
5′ Cn₁AAATGn₂GAn₃n₄GCTAGGn₅GGn₆n₇GGAATCTGAG 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides;
d) in a preferred embodiment the central stretch of nucleotides comprises the sequence

[SEQ ID NO: 178]
5′ Gn₁AAATGn₂GAn₃n₄GCTAGGn₅GGn₆n₇GGAATCTGAG 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides;
wherein in a more preferred embodiment the central stretch of nucleotides comprises the sequence

[SEQ ID NO: 178]
5′ Gn₁AAATGn₂GAn₃n₄GCTAGGn₅GGn₆n₇GGAATCTGAG 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides; or the sequence

[SEQ ID NO: 177]
5′ Cn₁AAATGn₂GAn₃n₄GCTAGGn₅GGn₆n₇GGAATCTGAG 3′,

wherein n₁ is G or rG, n₂ is G or rG, n₃ is G or rG, n₄ is G or rG, n₅ is T or rT, n₆ is A or rA, n₇ is A or rA, and wherein G, A, T and C are 2′-deoxyribonucleotides, and rG, rA and rT are ribonucleotides.

Glucagon binding nucleic acid molecules 257-E1-R09-001, 257-E1-R15-001, 257-E1-R18-001, 257-E1-R19-001, 257-E1-R29-001, 257-E1-R30-001, 257-E1-R15/29-001, 257-E1-R29/30-001, 257-E1-R15/29/30, 257-E1-R18/29/30-001, 257-E1-R15/18/29/30-001, 257-E1-7xR-023, 257-E1-6xR-001 and truncated derivatives thereof (257-E1-6xR-003 . . . 257-E1-6xR-020 and 257-E1-6xR-029 257-E1-6xR-033; 257-E1-7xR-037, see FIGS. 3A, 3B and 3C) showed the best binding affinity to glucagon and comprise the following sequences for the central stretch of nucleotides:
a) 257-E1-R09-001:

[SEQ ID NO: 179]
5′ GrGAAATGGGAGGGCTAGGTGGAAGGAATCTGAG 3′,

wherein G, A, T and C are 2′-deoxyribonucleotides, and rG is a ribonucleotide;
b) 257-E1-R15-001:

[SEQ ID NO: 180]
5′ GGAAATGrGGAGGCTAGGTGGAAGGAATCTGAG 3′,

wherein G, A, T and C are 2′-deoxyribonucleotides, and rG is a ribonucleotide;
c) 257-E1-R18-001:

[SEQ ID NO: 181]
5′ GGAAATGGGArGGGCTAGGTGGAAGGAATCTGAG 3′,

wherein G, A, T and C are 2′-deoxyribonucleotides, and rG is a ribonucleotide;
d) 257-E1-R19-001:

[SEQ ID NO: 182]
5′ GGAAATGGGAGrGGCTAGGTGGAAGGAATCTGAG 3′,

wherein G, A, T and C are 2′-deoxyribonucleotides, and rG is a ribonucleotide;
e) 257-E1-R29-001:

[SEQ ID NO: 183]
5′ GGAAATGGGAGGGCTAGGTGGrAAGGAATCTGAG 3′,

wherein G, A, T and C are 2′-deoxyribonucleotides, and rA is a ribonucleotide;
f) 257-E1-R30-001:

[SEQ ID NO: 184]
5′ GGAAATGGGAGGGCTAGGTGGArAGGAATCTGAG 3′,

wherein G, A, T and C are 2′-deoxyribonucleotides, and rA is a ribonucleotide;
g) 257-E1-R15/29-001:

[SEQ ID NO: 185]
5′ GGAAATGrGGAGGGCTAGGTGGrAAGGAATCTGAG 3′,

wherein G, A, T and C are 2′-deoxyribonucleotides, and rG is a are ribonucleotides;
h) 257-E1-R29/30-001:

[SEQ ID NO: 186]
5′ GGAAATGGGAGGGCTAGGTGGrArAGGAATCTGAG 3′,

wherein G, A, T and C are 2′-deoxyribonucleotides, and rA is a ribonucleotide;
i) 257-E1-R15/29/30-001:

[SEQ ID NO: 187]
5′ GcAAATGrGGAGGGCTAGGTGGrArAGGAATCTGAG 3′,

wherein G, A, T and C are 2′-deoxyribonucleotides, and rG and rA are ribonucleotides;
j) 257-E1-R18/29/30-001:

[SEQ ID NO: 188]
5′ GGAAATGGGArGGGCTAGGTGGrArAGGAATCTGAG 3′,

wherein X₁ is G, X₂ is G, X₃ is rG, X₄ is G, X₅ is T, X₆ is rA, X₇ is rA, and wherein G, A, T and C are 2′-deoxyribonucleotides, and rG and rA are ribonucleotides;
k) 257-E1-R15/18/29/30-001:

[SEQ ID NO: 189]
5′ GGAAATGrGGArGGGCTAGGTGGrArAGGAATCTGAG 3′,

wherein G, A, T and C are 2′-deoxyribonucleotides, and rG and rA are ribonucleotides;
l) 257-E1-6xR-001:

[SEQ ID NO: 190]
5′ GrGAAATGrGGArGrGGCTAGGTGGrArAGGAATCTGAG 3′,

wherein G, A, T and C are 2′-deoxyribonucleotides, and rG and rA are ribonucleotides;
m) 257-E1-7xR-023:

5′ GrGAAATGrGGArGrGGCTAGGrTGGrArAGGAATCTGAG 3′,

wherein G, A, T and C are 2′-deoxyribonucleotides, and rG, rA and rT are ribonucleotides.

As shown above, glucagon binding nucleic acid 257-E1-001 consists of 2′-deoxyribonucleotides and deletion of nucleotides of the first and second terminal stretch of nucleotides of 257-E1-001 led to reduced binding affinity (see FIG. 2A, 257-E1-002, 257-E1-003, 257-E1-004, 257-E1-004 and 257-E1-005).

Surprisingly, for glucagon binding nucleic acid 257-E1-6xR-001 that comprises a central stretch of nucleotides with six ribonucleotides instead of 2′-deoxyribonucleotides the inventors could show that the truncation of the first and the second terminal stretch of nucleotides from seven nucleotides (see 257-E1-6xR-001, FIG. 3A) to six nucleotides (see 257-E1-6xR-008/-010/-011/-012/-013/-016/-018/, FIGS. 3A and 3B) and five nucleotides (see 257-E1-6xR-020, FIG. 3C) did not lead to a reduction of binding affinitiy. Derivates of glucagon binding nucleic acid 257-E1-6xR-001 comprising terminal stretches with less than five nucleotides showed reduced binding affinity to glucagon: 257-E1-6xR-029 with a first and a second terminal stretch of nucleotides each with four nucleotides; 257-E1-6xR-030 and 257-E1-6xR-031 with a first and a second terminal stretch of nucleotides each with three nucleotides; 257-E1-6xR-032 with a first and a second terminal stretch of nucleotides each with two nucleotides; and 257-E1-6xR-033 with a first and a second terminal stretch of nucleotides each with one nucleotide (see FIG. 3C).

In order to further truncate glucagon binding nucleic acid molecule 257-E1-6xR-010 while maintaining the binding affinity to glucagon the 2′-deoxyribonucleotide at position 19 of the central stretch of nucleotides was substituted by a ribonucleotide leading to the glucagon binding nucleic acid 257-E1-7xR-023. Both molecules, glucagon binding nucleic acid molecule 257-E1-6xR-010 and glucagon binding nucleic acid molecule 257-E1-7xR-023 showed similar binding affinities to glucagon (FIGS. 3A and 3C). Suprisingly, the inventors could show that a molecule comprising the identical central stretch of nucleotides and a first and a second terminal stretch of nucleotides each with three nucleotides (see glucagon binding nucleic acid molecule 257-E1-7xR-037), has almost the same binding affinity to glucagon as glucagon binding nucleic acid molecule 257-E1-7xR-023 with a first and a second terminal stretch of six nucleotides, respectively (see FIG. 3C).

The first and the second terminal stretches of glucagon binding nucleic acid molecules of Type A comprises one (see 257-E1-6xR-033), two (see 257-E1-6xR-032), three (e.g. 257-E1-6xR-030 or 257-E1-7xR-037), four (see 257-E1-6xR-029), five (e.g. 257-E1-6xR-020), six (e.g. 257-E1-6xR-010) or seven (e.g. 257-E1-RxR-001 or 257-E1-E1-001) nucleotides (FIG. 1 to FIG. 3), whereby the stretches optionally hybridize with each other, whereby upon hybridization a double-stranded structure is formed. This double-stranded structure can consist of one to seven basepairs. However, such hybridization is not necessarily given in the molecule.

Combining the first terminal stretches of nucleotides and the second terminal stretches of nucleotides of all tested glucagon binding nucleic acid molecules of Type A the generic formula for the first terminal stretch of nucleotides is 5′ Z₁Z₂Z₃Z₄Z₅Z₆V 3′ and the generic formula for the second terminal stretch of nucleotides is 5′ BZ₇Z₈Z₉Z₁₀Z₁₁Z₁₂ 3′, wherein Z₁ is G or absent, Z₂ is S or absent, Z₃ is V or absent, Z₄ is B or absent, Z₅ is B or absent, Z₆ is R or absent, Z₇ is B or absent, Z₈ is V or absent, Z₉ is V or absent, Z₁₀ is B or absent, Z₁₁ is S or absent, and Z₁₂ is C or absent, whereby

in a first preferred embodiment
d) Z₁ is G, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is C, or
e) Z₁ is absent, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is C, or
f) Z₁ is G, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is absent, and
   in a second preferred embodiment
a) Z₁ is absent, Z₂ is S, Z₃ is V, Z₄ is B, Z₅ is B, Z₆ is V, Z₇ is B, Z₈ is V, Z₉ is V, Z₁₀ is B, Z₁₁ is S, and Z₁₂ is absent, or
b) Z₁ is absent, Z₂ is S, Z₃ is V, Z₄ is B, Z₅ is B, Z₆ is V, Z₇ is B, Z₈ is V, Z₉ is C, Z₁₀ is B, Z₁₁ is absent, and Z₁₂ is absent, or
c) Z₁ is absent, Z₂ is absent, Z₃ is V, Z₄ is B, Z₅ is B, Z₆ is V, Z₇ is B, Z₈ is V, Z₉ is C, Z₁₀ is B, Z₁₁ is S, and Z₁₂ is absent, and
   in a third preferred embodiment
d) Z₁ is absent, Z₂ is absent, Z₃ is V, Z₄ is B, Z₅ is B, Z₆ is V, Z₇ is B, Z₈ is V, Z₉ is V, Z₁₀ is B, Z₁₁ is absent, and Z₁₂ is absent, or
e) Z₁ is absent, Z₂ is absent, Z₃ is V, Z₄ is B, Z₅ is B, Z₆ is V, Z₇ is B, Z₈ is V, Z₉ is V, Z₁₀ is absent, Z₁₁ is absent, and Z₁₂ is absent, or
f) Z₁ is absent, Z₂ is absent, Z₃ is absent, Z₄ is B, Z₅ is B, Z₆ is V, Z₇ is B, Z₈ is V, Z₉ is V, Z₁₀ is B, Z₁₁ is absent, and Z₁₂ is absent, and
   in a fourth preferred embodiment
d) Z₁ is absent, Z₂ is absent, Z₃ is absent, Z₄ is B, Z₅ is B, Z₆ is V, Z₇ is B, Z₈ is V, Z₉ is V, Z₁₀ is absent, Z₁₁ is absent, and Z₁₂ is absent, or
e) Z₁ is absent, Z₂ is absent, Z₃ is absent, Z₄ is B, Z₅ is B, Z₆ is V, Z₇ is B, Z₈ is V, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is B, Z₆ is V, Z₇ is B, Z₈ is V, Z₉ is V, Z₁₀ is absent, Z₁₁ is absent, and Z₁₂ is absent, and
   in a fifth preferred embodiment
d) Z₁ is absent, Z₂ is absent, Z₃ is absent, Z₄ is absent, Z₅ is B, Z₆ is V, Z₇ is B, Z₈ is V, Z₉ is absent, Z₁₀ is absent, Z₁₁ is absent, and Z₁₂ is absent, or
e) Z₁ is absent, Z₂ is absent, Z₃ is absent, Z₄ is absent, Z₅ is B, Z₆ is V, Z₇ is B, Z₈ is absent, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is absent, Z₆ is V, Z₇ is B, Z₈ is V, Z₉ is absent, Z₁₀ is absent, Z₁₁ is absent, and Z₁₂ is absent, and
   in sixth preferred embodiment
e) Z₁ is absent, Z₂ is absent, Z₃ is absent, Z₄ is absent, Z₅ is absent, Z₆ is V, Z₇ is B, Z₈ is absent, Z₉ is absent, Z₁₀ is absent, Z₁₁ is absent, and Z₁₂ is absent,
f) Z₁ is absent, Z₂ is absent, Z₃ is absent, Z₄ is absent, Z₅ is absent, Z₆ is V, Z₇ is absent, Z₈ is absent, Z₉ is absent, Z₁₀ is absent, Z₁₁ is absent, and Z₁₂ is absent,
g) Z₁ is absent, Z₂ is absent, Z₃ is absent, Z₄ is absent, Z₅ is absent, Z₆ is absent, Z₇ is B, Z₈ is absent, Z₉ is absent, Z₁₀ is absent, Z₁₁ is absent, and Z₁₂ is absent,
h) Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent,

Combining the first terminal stretches of nucleotides and the second terminal stretches of nucleotides of glucagon binding nucleic acid molecules 257-A1-001, 257-D4-001, 257-F4-001, 257-B3-001, 257-D3-001, 257-E4-001, 257-C4-001, 257-C1-001, 257-H2-001, 257-E1-001, 257-E1-R9-001, 257-E1-R15-001, 257-E1-R18-001, 257-E1-R19-001, 257-E1-R29-001, 257-E1-R30-001, 257-E1-R15/29-001, 257-E1-R29/30-001, 257-E1-R15/29/30-001, 257-E1-R18/29/30-001, 257-E1-R15/18/29/30-001 and 257-E1-6xR-001 the generic formula for the first terminal stretch of nucleotides is 5′ Z₁Z₂Z₃Z₄Z₅Z₆V 3′ and the generic formula for the second terminal stretch of nucleotides is 5′ BZ₇Z₈Z₉Z₁₀Z₁₁Z₁₂, wherein
d) Z₁ is G, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is C, or
e) Z₁ is absent, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is C, or
f) Z₁ is G, Z₂ is C, Z₃ is R, Z₄ is B, Z₅ is Y, Z₆ is R, Z₇ is Y, Z₈ is R, Z₉ is V, Z₁₀ is Y, Z₁₁ is G, and Z₁₂ is absent,
   wherein the glucagon binding nucleic acid molecules with the best binding affinity to glucagon comprise the following combinations of the first terminal stretch and the second terminal stretch of nucleotides:
g) 257-A1-001: 5′ GCACTGG 3′(first terminal stretch of nucleotides) and 5′ GCAGTGC 3′ (second terminal stretch of nucleotides), or
h) 257-F4-001: 5′ GCACTGA 3′ (first terminal stretch of nucleotides) and 5′ GCAGTGC 3′ (second terminal stretch of nucleotides), or
i) 257-E4-001: 5′ GCAGTGG 3′ (first terminal stretch of nucleotides) 5′ TCACTGC 3′ (second terminal stretch of nucleotides), or
j) 257-E1-001: 5′ GCAGTGG 3′ (first terminal stretch of nucleotides) 5′ CTACTGC 3′ (second terminal stretch of nucleotides), or
k) 257-C1-001: 5′ GCGCTGG 3′ (first terminal stretch of nucleotides) 5′ GCAGTGC 3′ (second terminal stretch of nucleotides), or
l) 257-H2-001: 5′ GCGCCAG 3′ (first terminal stretch of nucleotides) 5′ TCGGCGC 3′ (second terminal stretch of nucleotides).

Combining the first terminal stretches of nucleotides and the second terminal stretches of nucleotides of glucagon binding nucleic acid molecules 257-E1-002, 257-E1-003, 257-E1-6xR-003, 257-E1-6xR-005, 257-E1-6xR-006, 257-E1-6xR-007, 257-E1-6xR-008, 257-E1-6xR-009, 257-E1-6xR-010, 257-E1-6xR-011, 257-E1-6xR-012, 257-E1-6xR-013, 257-E1-6xR-014, 257-E1-6xR-015, 257-E1-6xR-016, 257-E1-6xR-017, 257-E1-6xR-018 and 257-E1-7xR-023 the generic formula for the first terminal stretch of nucleotides is 5′ Z₁Z₂Z₃Z₄Z₅Z₆G 3′ and the generic formula for the second terminal stretch of nucleotides is 5′ CZ₇Z₈Z₉Z₁₀Z₁₁Z₁₂ 3 wherein
d) Z₁ is absent, Z₂ is S, Z₃ is V, Z₄ is G, Z₅ is Y, Z₆ is S, Z₇ is B, Z₈ is R, Z₉ is C, Z₁₀ is B, Z₁₁ is S, and Z₁₂ is absent, or
e) Z₁ is absent, Z₂ is S, Z₃ is V, Z₄ is G, Z₅ is Y, Z₆ is S, Z₇ is B, Z₈ is R, Z₉ is C, Z₁₀ is B, Z₁₁ is absent, and Z₁₂ is absent, or
f) Z₁ is absent, Z₂ is absent, Z₃ is V, Z₄ is G, Z₅ is Y, Z₆ is S, Z₇ is B, Z₈ is R, Z₉ is C, Z₁₀ is B, Z₁₁ is S, and Z₁₂ is absent,
   wherein the glucagon binding nucleic acid molecules with the best binding affinity to glucagon comprise the following combinations of the first terminal stretch and the second terminal stretch of nucleotides:
h) 257-E1-6xR-008: 5′ GCGCGG 3′ (first terminal stretch of nucleotides) and 5′ CTGCGC 3′(second terminal stretch of nucleotides), or.
i) 257-E1-6xR-010: 5′ GCGCGG 3′ (first terminal stretch of nucleotides) and 5′ CCGCGC 3′(second terminal stretch of nucleotides), or
j) 257-E1-6xR-011: 5′ GGGCCG 3′ (first terminal stretch of nucleotides) and 5′ CGGCCC 3′(second terminal stretch of nucleotides), or
k) 257-E1-6xR-012: 5′ GCGCCG 3′ (first terminal stretch of nucleotides) and 5′ CGGCGC 3′(second terminal stretch of nucleotides), or
l) 257-E1-6xR-013: 5′ GAGCGG 3′ (first terminal stretch of nucleotides) and 5′ CCGCTC 3′ (second terminal stretch of nucleotides), or
m) 257-E1-6xR-016: 5′ GCGTGG 3′ (first terminal stretch of nucleotides) and 5′ CCACGC 3′(second terminal stretch of nucleotides), or
n) 257-E1-6xR-018: 5′ GCGTCG 3′ (first terminal stretch of nucleotides) and 5′ CGACGC 3′ (second terminal stretch of nucleotides).

Combining the first terminal stretches of nucleotides and the second terminal stretches of nucleotides of glucagon binding nucleic acid, molecules 257-E1-6xR-004, 257-E1-6xR-019 and 257-E1-6xR-020 the generic formula for the first terminal stretch of nucleotides is 5′ Z₁Z₂Z₃Z₄Z₅Z₆G 3′ and the generic formula for the second terminal stretch of nucleotides is of 5′ CZ₇Z₈Z₉Z₁₀Z₁₁Z₁₂ 3′, wherein
d) Z₁ is absent, Z₂ is absent, Z₃ is V, Z₄ is G, Z₅ is Y, Z₆ is G, Z₇ is Y, Z₈ is R, Z₉ is C, Z₁₀ is B, Z₁₁ is absent, and Z₁₂ is absent, or
e) Z₁ is absent, Z₂ is absent, Z₃ is V, Z₄ is G, Z₅ is Y, Z₆ is G, Z₇ is Y, Z₈ is R, Z₉ is C, Z₁₀ is absent, Z₁₁ is absent, and Z₁₂ is absent, or
f) Z₁ is absent, Z₂ is absent, Z₃ is absent, Z₄ is G, Z₅ is Y, Z₆ is G, Z₇ is Y, Z₈ is R, Z₉ is C, Z₁₀ is B, Z₁₁ is absent, and Z₁₂ is absent,
   wherein the glucagon binding nucleic acids with the best binding affinity to glucagon comprise the following combinations of the first terminal stretch and the second terminal stretch of nucleotides:
c) 257-E1-6xR-019: 5′ GGCGG 3′ (first terminal stretch of nucleotides) and 5′ CCGCC 3′ (second terminal stretch of nucleotides), or
d) 257-E1-6xR-020: 5′ CGCGG 3′ (first terminal stretch of nucleotides) and 5′ CCGCG 3′ (second terminal stretch of nucleotides).

Combining the first terminal stretches of nucleotides and the second terminal stretches of nucleotides of glucagon binding nucleic acid molecule 257-E1-6xR-029 and 257-E1-005 the generic formula for the first terminal stretch of nucleotides is 5′ Z₁Z₂Z₃Z₄Z₅Z₆G 3′ and the generic formula for the second terminal stretch of nucleotides is 5′ CZ₇Z₈Z₉Z₁₀Z₁₁Z₁₂ 3′, wherein
d) Z₁ is absent, Z₂ is absent, Z₃ is absent, Z₄ is G, Z₅ is Y, Z₆ is G, Z₇ is Y, Z₈ is R, Z₉ is C, Z₁₀ is absent, Z₁₁ is absent, and Z₁₂ is absent, or
e) Z₁ is absent, Z₂ is absent, Z₃ is absent, Z₄ is G, Z₅ is Y, Z₆ is G, Z₇ is Y, Z₈ is R, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is Y, Z₆ is G, Z₇ is Y, Z₈ is R, Z₉ is C, Z₁₀ is absent, Z₁₁ is absent, and Z₁₂ is absent,
   wherein the glucagon binding nucleic acid molecule with the best binding affinity to glucagon comprises the following combinations of the first terminal stretch and the second terminal stretch of nucleotides:
257-E1-6xR-029: 5′ GCGG 3′ (first terminal stretch of nucleotides) and 5′ CCGC 3′ (second terminal stretch of nucleotides).

Combining the first terminal stretches of nucleotides and the second terminal stretches of nucleotides of glucagon binding nucleic acid molecules 257-E1-6xR-030, 257-E1-6xR-031 and 257-E1-7xR-037 the generic formula for the first terminal stretch of nucleotides is 5′ Z₁Z₂Z₃Z₄Z₅Z₆G 3′ and the generic formula for the second terminal stretch of nucleotides is 5′ CZ₇Z₈Z₉Z₁₀Z₁₁Z₁₂ 3′, wherein
d) Z₁ is absent, Z₂ is absent, Z₃ is absent, Z₄ is absent, Z₅ is S, Z₆ is S, Z₇ is S, Z₈ is S, Z₉ is absent, Z₁₀ is absent, Z₁₁ is absent, and Z₁₂ is absent, or
e) Z₁ is absent, Z₂ is absent, Z₃ is absent, Z₄ is absent, Z₅ is S, Z₆ is S, Z₇ is S, Z₈ is absent, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is absent, Z₆ is S, Z₇ is S, Z₈ is S, Z₉ is absent, Z₁₀ is absent, Z₁₁ is absent, and Z₁₂ is absent,
   wherein the glucagon binding nucleic acid molecule with the best binding affinity to glucagon comprise the following combinations of the first terminal stretch and the second terminal stretch of nucleotides:
257-E1-6xR-030: 5′ GCG 3′ (first terminal stretch of nucleotides) and 5′ CGC 3′ (second terminal stretch of nucleotides).

Combining the first terminal stretches of nucleotides and the second terminal stretches of nucleotides of glucagon binding nucleic acid molecules 257-E1-6xR-032 and 257-E1-6xR-033 the generic formula for the first terminal stretch of nucleotides is 5′ Z₁Z₂Z₃Z₄Z₅Z₆G 3′ and the generic formula for the second terminal stretch of nucleotides is 5′ CZ₇Z₈Z₉Z₁₀Z₁₁Z₁₂ 3′,

wherein
c) Z₁ is absent, Z₂ is absent, Z₃ is absent, Z₄ is absent, Z₅ is absent, Z₆ is G, Z₇ is C, Z₈ is absent, Z₉ is absent, Z₁₀ is absent, Z₁₁ is absent, and Z₁₂ is absent (see 257-E1-6xR-032), or
d) Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent (see 257-E1-6xR-033).

In order to prove the functionality of glucagon binding nucleic acid molecules 257-E1-6xR-001, 257-E1-6xR-030 and 257-E1-7xR-037 were synthesized as spiegelmers. For PEGylation Spiegelmers 257-E1-6xR-030 and 257-E1-7xR-037 were synthesized with an amino-group at its 5′-end. To the amino-modified spiegelmers 257-E1-6xR-030-5′amino [SEQ ID NO: 158] and 257-E1-7xR-037-5′amino [SEQ ID NO: 159] a 40 kDa PEG-moiety was coupled leading to glucagon binding spiegelmers 257-E1-6xR-030-5′-PEG (also referred to as NOX-G15) [SEQ ID NO: 91] and 257-E1-7xR-037-5′-PEG (also referred to as NOX-G16) [SEQ ID NO: 92]. Synthesis and PEGylation of the spiegelmer is described in Example 2.

Glucagon binding spiegelmers 257-E1-6xR-001, 257-E1-7xR-037, NOX-G15 and NOX-G16 were able to inhibit/antagonize in vitro the function of glucagon to its receptor with an IC₅₀ of 2-3 nM (FIG. 17: NOX-G15 and NOX-G16; FIG. 20 A: 257-E1-6xR-001, 257-E1-7xR-0037, NOX-G15 and NOX-G16; for protocol of the in vitro assay see Example 5).

As shown in Example 8, glucagon binding spiegelmer NOX-G 15 was effective in a glucose tolerance test in a type 1 DM and in a type 2 DM animal experiment (FIGS. 23 and 24).

Furthermore, as shown in example 6 the binding selectivity of the glucagon binding spiegelmers 257-E1-6xR-001, 257-E1-7xR-0037, NOX-G15 and NOX-G16 was determined (FIGS. 19 and 20).

### 1.2 Glucagon Binding Nucleic Acid Molecules of Type B

As depicted in FIG. 4 to FIG. 6 glucagon binding nucleic acid molecules of Type B comprise one central stretch of nucleotides defining a potential glucagon binding motif.

In general, glucagon binding nucleic acid molecules of Type B comprise at the 5′-end and the 3′-end terminal stretches of nucleotides: the first terminal stretch of nucleotides and the second terminal stretch of nucleotides. The first terminal stretch of nucleotides and the second terminal stretch of nucleotides can hybridize to each other, whereby upon hybridization a double-stranded structure is formed. However, such hybridization is not necessarily given in the molecule.

The three stretches of nucleotides of glucagon binding nucleic acid molecules of Type B—a first terminal stretch of nucleotides, a central stretch of nucleotides and a second terminal stretch of nucleotides—are arranged in 5′→3′-direction as follows: the first terminal stretch of nucleotides—the central stretch of nucleotides—the second terminal stretch of nucleotides. Alternatively, however, the first terminal stretch of nucleotides, the central stretch of nucleotides and the second terminal stretch of nucleotides are arranged to each other in 5′→3′-direction as follows: the second terminal stretch of nucleotides—the central stretch of nucleotides—the first terminal stretch of nucleotides.

The sequences of the defined stretches may be different between the glucagon binding nucleic acid molecules of Type B which influences the binding affinity to glucagon. Based on binding analysis of the different glucagon binding nucleic acid molecules of Type B the central stretch of nucleotides and their nucleotide sequences as described in the following are individually and more preferably in their entirety essential for binding to human glucagon.

The glucagon binding nucleic acid molecules of Type B according to the present invention are shown in FIGS. 4 to 6. All of them were tested as aptamers and/or spiegelmers for their ability to bind glucagon. The first glucagon binding nucleic acid molecule of Type B that was characterized for its binding affinity to glucagon was nucleic acid molecule 259-H6-001 that consists of deoxyribonucleotides. The equilibrium binding constant K_{D} of nucleic acid molecule 259-H6-001 was determined as aptamer by direct pull-down binding assays (K_{D}_{_{—}}ₐₚₜₐₘₑᵣ=33 nM, FIG. 4).

Glucagon binding nucleic acid molecules 259-D5-001, 259-B7-001, 259-B8-001, 259-A5-001, 259-C8-001, 259-E5-001, 259-E7-001 and 259-F5-001—also consisting of 2′-deoxyribonucleotides—were tested as aptamers in comparative competition pull-down assays vs. glucagon binding nucleic acid 259-H6-001. Glucagon binding nucleic acid molecule 259-C8-001 showed similar binding affinity as 259-H6-001, whereby both molecules comprise a central stretch of 32 nucleotides with the sequence of

[SEQ ID NO: 212]
5′-AGGAAAGGTTGGTAAAGGTTCGGTTGGATTCA-′3.

binding nucleic acid molecules 259-D5-001 and 259-B7-001 have minor changes in the sequence of the central stretch of nucleotides and showed weaker binding affinity in comparison to glucagon binding nucleic acid molecule 259-H6-001. Also, glucagon binding nucleic acid molecules 259-B8-001, 259-A5-001, and 259-E5-001 have minor changes in the sequence of the central stretch of nucleotides and showed much weaker binding affinity in comparison to glucagon binding nucleic acid molecule 259-H6-001. Glucagon binding nucleic acids 259-F5-001 and 259-E7-001 comprise each a central stretch of 29 nucleotides that is related to central stretch of glucagon binding nucleic acid molecule 259-H6-001 and showed weaker and much weaker binding affinity in comparison to glucagon binding nucleic acid molecule 259-H6-001 (FIG. 4). The central stretches of 259-F5-001

[SEQ ID NO: 198]
(5′-AGAAGGTTGGTAAGTTTCGGTTGGATCTG-′3)

### and 259-E7-001

[SEQ ID NO: 199]
(5′-AGAAGGTCGGTAAGTTTCGGTAGGATCTG-′3)

comprises two substretches that are related to the substretches in the central stretch of glucagon binding nucleic acid molecule 259-H6-001 (first substretch:

[SEQ ID NO: 213]
5′-AAGGTTGGTA-′3,

second substretch:

5′-AGGTTCGGTTGGAT-′3 [SEQ ID NO: 214]):

259-F5-001: first substretch:

[SEQ ID NO: 213]
5′-AAGGTTGGTA-′3,

second substretch:

[SEQ ID NO: 215]
5′-AGTTTCGGTTGGAT-′3;

259-E7-001: first substretch:

[SEQ ID NO: 216]
5′-AAGGTCGGTA-′3,

second substretch:

[SEQ ID NO: 217]
5′-AGTTTCGGTAGGAT-′3.

Derivatives 259-H6-002, 259-H6-005, 259-H6-003 and 259-H6-004 of glucagon binding nucleic molecule 259-H6-001 consist of 2′-deoxyribonucleotides and comprise first and second terminal stretches of nucleotides with seven, six, five or three nucleotides, whereby glucagon binding nucleic molecule 259-H6-001 comprises a first and second terminal stretch of nucleotides each with nine nucleotides. Derivatives 259-H6-002 and 259-H6-005 of glucagon binding nucleic molecule 259-H6-001 showed similar binding affinity in a comparative competition pull-down assay as glucagon binding nucleic molecule 259-H6-001. Derivatives 259-H6-003 and 259-H6-004 of glucagon binding nucleic molecule 259-H6-001 showed reduced binding affinity in a comparative competition pull-down assay compared to glucagon binding nucleic molecule 259-H6-001 (FIG. 5). Accordingly, deletion of more than three nucleotides of the first and of the second terminal stretch of nucleotides of glucagon binding nucleic acid molecule 259-H6-001 led to reduced binding affinity to glucagon.

As shown for glucagon binding nucleic acid molecules 259-E7-001 and 259-F5-001 a glucagon binding nucleic acid molecule with a central stretch of 29 nucleotides can bind to glucagon. The glucagon binding nucleic acid molecules 259-H6-006, 259-H6-007 and 259-H6-008 are derivatives of glucagon binding nucleic acid molecule 259-H6-002 (that has a central stretch of 32 nucleotides) and all comprise the same first and second terminal stretches of glucagon binding nucleic acid molecule 259-H6-002 and central stretches of nucleotides that are almost identical to the central stretch of glucagon binding nucleic acid molecule 259-H6-002. Due to deletion of one or two nucleotides within the central stretch as described for glucagon binding nucleic acid molecule 259-H6-002 the central stretch consist of 31 or 30 nucleotides:

259-H6-006: central stretch of nucleotides:

[SEQ ID NO: 218]
5′-AGGA-AGGTTGGTAAAGGTTCGGTTGGATTCA-′3,

259-H6-007: central stretch of nucleotides:

[SEQ ID NO: 219]
5′-AGGAAAGGTTGGTAAGGTTCGGTTGGATTCA-′3,

259-H6-008: central stretch of nucleotides:

[SEQ ID NO: 220]
5′-AGGA-AGGTTGGTAAGGTTCGGTTGGATTCA-′3.

In a comparative competition pull-down assay versus glucagon binding nucleic acid molecule 259-H6-002 it was shown that the deletion of one (see 259-H6-006 and 259-H6-007) or two (see 259-H6-008) nucleotides of the central stretch of nucleotides of 259-H6-002 led to a reduction of binding affinity (FIG. 5).

However, combining the central stretches of nucleotides of glucagon binding nucleic acid molecules 259-D5-001, 259-H6-001, 259-B7-001, 259-B8-001, 259-A5-001, 259-C8-001, 259-E5-001, 259-E7-001, 259-F5-001, 259-H6-002, 259-H6-005, 259-H6-003, 259-H6-004, 259-H6-006, 259-H6-007 and 259-H6-008 these glucagon binding nucleic acid molecules comprise a central stretch of nucleotides consisting of 29, 30, 31 or 32 nucleotides selected from the group consisting of

(259-D5-001, 259-H6-001, 259-B7-001, 259-B8-001,
259-A5-001, 259-C8-001, 259-E5-001)
[SEQ ID NO: 221]
5′-AKGARAKGTTGSYAWAGRTTCGGTTGGATTCA-′3,
(259-F5-001)
[SEQ ID NO: 198]
5′-AGAAGGTTGGTAAGTTTCGGTTGGATCTG-′3,
(259-E7-001)
[SEQ ID NO: 199]
5′-AGAAGGTCGGTAAGTTTCGGTAGGATCTG-′3,
(259-H6-006)
[SEQ ID NO: 218]
5′-AGGAAGGTTGGTAAAGGTTCGGTTGGATTCA-′3,
(259-H6-007)
[SEQ ID NO: 219]
5′-AGGAAAGGTTGGTAAGGTTCGGTTGGATTCA-′3,
(259-H6-008)
[SEQ ID NO: 220]
5′-AGGAAGGTTGGTAAGGTTCGGTTGGATTCA-′3.

Glucagon binding nucleic acid molecules 259-H6-001 and 259-C8-001 showed the best binding affinity to glucagon and comprise the following sequences for the central stretch:

[SEQ ID NO: 212]
5′-AGGAAAGGTTGGTAAAGGTTCGGTTGGATTCA-′3.

The inventors surprisingly showed in comparative competition pull-down assays or by surface plasmon resonance analysis that the binding affinity of glucagon binding nucleic acid molecule 259-H6-002 was improved by replacing 2′-deoxyribonucleotides by ribonucleotides within the sequence of the central stretch of nucleotides. The 2′-deoxyribonucleotides and ribonucleotides are shown in FIGS. 29 and 30A-B, wherein in Example 1.2 and in the corresponding figures the following abbreviations were used: G is 2′deoxy-guanosine(5′monophosphate), C is 2′deoxy-cytidine(5′monophosphate), A is 2′deoxy-adenosine(5′monophosphate), T is 2′deoxy-thymidine(5′monophosphate), rG is guanosine(5′monophosphate), rU is uridine(5′monophosphate) and rA is adenosine(5′monophosphate). In particular replacing up to five 2′-deoxyribonucleotides by ribonucleotides in the central stretch of nucleotides of glucagon binding nucleic acid molecule 259-H6-002 resulted in improved binding affinity to glucagon by a factor of up to more than 22. In more detail, the inventors have surprisingly found that
a) replacing one 2′-deoxyribonucleotide by one ribonucleotide at position 6, 17 or 29 in the central stretch of nucleotides of glucagon binding nucleic acid molecule 259-H6-002 resulted in improved binding affinity to glucagon in comparison to the binding affinity of glucagon binding nucleic acid molecule 259-H6-002 (see FIGS. 6A, 6B and 6C; 259-H6-002-R13, 259-H6-002-R24, 259-H6-002-R36, 259-H6-005-R12, 259-H6-009-R12, 259-H6-010-R12, 259-H6-011-R12, 259-H6-012-R12, 259-H6-013-R12, 259-H6-014-R12, 259-H6-015-R12, 259-H6-016-R12);
b) replacing two 2′-deoxyribonucleotides by two ribonucleotides at positions 6 and 17, or 6 and 29, or 17 and 29 in the central stretch of nucleotides of glucagon binding nucleic acid molecule 259-H6-002 resulted in improved binding affinity to glucagon in comparison to the binding affinity of glucagon binding nucleic acid molecule 259-H6-002 (see FIG. 6A; 259-H6-002-R13/24, 259-H6-002-R13/36, 259-H6-002-R24/36);
c) replacing three 2′-deoxyribonucleotides by three ribonucleotides at positions 6, 17 and 29 in the central stretch of nucleotides of glucagon binding nucleic acid molecule 259-H6-002 resulted in improved binding affinity to glucagon in comparison to the binding affinity of glucagon binding nucleic acid molecule 259-H6-002 (see FIGS. 6A and 6C; 259-H6-002-R13/24/36 and 259-H6-014-R12/23/35); and
d) replacing five 2′-deoxyribonucleotides by five ribonucleotides at positions 6, 17, 23, 29 and 32 in the central stretch of nucleotides of glucagon binding nucleic acid molecule 259-H6-002 resulted in improved binding affinity to glucagon in comparison to the binding affinity of glucagon binding nucleic acid molecule 259-H6-002 (see FIG. 6C; 259-H6-014-R12/23/29/35/38).

Based on the data shown that replacing 2′-deoxyribonucleotides by ribonucleotides at several positions of the central stretch of nucleotides of glucagon binding nucleic acid molecules of Type B led to improved binding to glucagon the central stretch of glucagon binding nucleic acid molecules 259-D5-001, 259-H6-001, 259-B7-001, 259-B8-001, 259-A5-001, 259-C8-001, 259-E5-001 can be summarized in the following generic formula

[SEQ ID NO: 197]
5′-AKGAR n₁KGTTGSYAWAn₂RTTCGn₃TTGGAn₄TCn₅-′3,

wherein n₁ is A or rA, n₂ is G or rG, n₃ is G or rG, n₄ is T or rU, n₅ is A or rA, and wherein G, A, T, C, K, Y, S, W and R are 2′-deoxyribonucleotides, and rG, rA and rU are ribonucleotides.

The glucagon binding nucleic acid molecules 259-H6-001, 259-C8-001, 259-H6-002-R13, 259-H6-002-R24, 259-H6-002-R36, 259-H6-005-R12, 259-H6-009-R12, 259-H6-010-R12, 259-H6-011-R12, 259-H6-012-R12, 259-H6-013-R12, 259-H6-014-R12, 259-H6-015-R12, 259-H6-016-R12, 259-H6-002-R13/24, 259-H6-002-R13/36, 259-H6-002-R24/36, 259-H6-002-R13/24/36, 259-H6-014-R12/23/35 and 259-H6-014-R12/23/35/38 showed better binding affinity to glucagon than other glucagon binding nucleic acid molecules of Type B and share the following sequences for the central stretch: 5′ AGGAAn₁GGTTGGTAAAn₂GTTCGn₃TTGGAn₄TCn₅ 3′ [SEQ ID NO: 203], wherein n₁ is A or rA, n₂ is G or rG, n₃ is G or rG, n₄ is T or rU, n₅ is A or rA, and wherein G, A, T, and C are 2′-deoxyribonucleotides, and rG, rA and rU are ribonucleotides.

The glucagon binding nucleic acid molecules 259-H6-002-R13, 259-H6-002-R24, 259-H6-002-R36, 259-H6-002-R13/24, 259-H6-002-R13/36, 259-H6-002-R13/24/36, 259-H6-014-R12/23/35, 259-H6-014-R12/23/29/35/38 showed the best binding affinity to glucagon and comprise the following sequences for the central stretch of nucleotides:
a) 259-H6-002-R13:

[SEQ ID NO: 204]
5′ AGGAArAGGTTGGTAAAGGTTCGGTTGGATTCA 3′,

wherein G, A, T, and C are 2′-deoxyribonucleotides; and rA is a ribonucleotide;
b) 259-H6-002-R24:

[SEQ ID NO: 205]
5′ AGGAAAGGTTGGTAAArGGTTCGGTTGGATTCA 3′,

wherein G, A, T, and C are 2′-deoxyribonucleotides, and rG is ribonucleotide;
c) 259-H6-002-R36:

[SEQ ID NO: 206]
5′ AGGAAAGGTTGGTAAAGGTTCGGTTGGArUTCA 3′,

wherein G, A, T, and C are 2′-deoxynucleotides, and rU is a ribonucleotide;
d) 259-H6-002-R13/24:

[SEQ ID NO: 207]
5′ AGGAArAGGTTGGTAAArGGTTCGGTTGGATTCA 3′,

wherein G, A, T, and C are 2′-deoxynucleotides, and rG and rA are ribonucleotides;
e) 259-H6-002-R13/36:

[SEQ ID NO: 208]
5′ AGGAArAGGTTGGTAAAGGTTCGGTTGGArUTCG 3′,

wherein G, A, T, and C are 2′-deoxynucleotides, and rA and rU are ribonucleotides;
f) 259-H6-002-R24/36:

[SEQ ID NO: 209]
5′ AGGAArAGGTTGGTAAAGGTTCGGTTGGArUTCA 3′,

wherein G, A, T, and C are 2′-deoxynucleotides, and rG is a rU are ribonucleotides;
g) 259-H6-002-R13/24/36 and 259-H6-014-R12/23/35:

[SEQ ID NO: 210]
5′ AGGAArAGGTTGGTAAArGGTTCGGTTGGArUTCA 3′,

and wherein G, A, T, and C are 2′-deoxyribonucleotides, and rG, rA and rU are ribonucleotides;
h) 259-H6-014-R12/23/29/35/38:

[SEQ ID NO: 211]
5′ AGGAArAGGTTGGTAAArGGTTCGrGTTGGArUTCrA 3′,

and wherein G, A, T, and C are 2′-deoxyribonucleotides, and rG, rA and rU are ribonucleotides.

The first and the second terminal stretches of glucagon binding nucleic acid molecules of Type B comprise three (see 259-H6-004), five (see 259-H6-003), six (e.g. 259-H6-005, 259-H6-005-R12, 259-H6-009-R12, 259-H6-010-R12, 259-H6-011-R12, 259-H6-012-R12), seven (e.g. 259-H6-002 and derivatives thereof such as 259-H6-002-R13, 259-H6-002-R13/24/36) or nine (e.g. 259-H6-001) nucleotides (FIGS. 4 to 6), whereby the stretches optionally hybridize with each other, whereby upon hybridization a double-stranded structure is formed. This double-stranded structure can consist of one to nine basepairs. However, such hybridization is not necessarily given in the molecule.

Combining the first terminal stretches of nucleotides and the second terminal stretches of nucleotides of all tested glucagon binding nucleic acid molecules of Type B the generic formula for the first terminal stretch of nucleotides is 5′ Z₁Z₂Z₃Z₄Z₅Z₆SAK 3′ and the generic formula for the second terminal stretch of nucleotides is 5′ CKVZ₇Z₈Z₉Z₁₀Z₁₁Z₁₂ 3′, wherein Z₁ is C or absent, Z₂ is G or absent, Z₃ is R or absent, Z₄ is B or absent, Z₅ is B or absent, Z₆ is S or absent, Z₇ is S or absent, Z₈ is V or absent, Z₉ is N or absent, Z₁₀ is K or absent, Z₁₁ is M or absent, and Z₁₂ is S or absent, wherein

in a first preferred embodiment
d) Z₁ is C, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is S, or
e) Z₁ is absent, Z₂ is G, Z₃ is R, Z₄ is B, Z₅ is B, Z₆ is S, Z₇ is S, Z₈ is N, Z₉ is V, Z₁₀ is K, Z₁₁ is M, and Z₁₂ is S, or
f) Z₁ is C, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is absent, and
   in a second preferred embodiment
d) Z₁ is absent, Z₂ is G, Z₃ is R, Z₄ is B, Z₅ is B, Z₆ is S, Z₇ is S, Z₈ is V, Z₉ is N, Z₁₀ is K, Z₁₁ is M, and Z₁₂ is absent, or
e) Z₁ is absent, Z₂ is G, Z₃ is R, Z₄ is B, Z₅ is B, Z₆ is S, Z₇ is S, Z₈ is V, Z₉ is N, Z₁₀ is K, Z₁₁ is absent, and Z₁₂ is absent, or
f) Z₁ is absent, Z₂ is absent, Z₃ is R, Z₄ is B, Z₅ is B, Z₆ is S, Z₇ is S, Z₈ is V, Z₉ is N, Z₁₀ is K, Z₁₁ is M, and Z₁₂ is absent, and
   in a third preferred embodiment
d) Z₁ is absent, Z₂ is absent, Z₃ is R, Z₄ is B, Z₅ is B, Z₆ is S, Z₇ is S, Z₈ is V, Z₉ is N, Z₁₀ is K, Z₁₁ is absent, and Z₁₂ is absent, or
e) Z₁ is absent, Z₂ is absent, Z₃ is R, Z₄ is B, Z₅ is B, Z₆ is S, Z₇ is S, Z₈ is V, Z₉ is N, Z₁₀ is absent, Z₁₁ is absent, and Z₁₂ is absent, or
f) Z₁ is absent, Z₂ is absent, Z₃ is absent, Z₄ is B, Z₅ is B, Z₆ is S, Z₇ is S, Z₈ is V, Z₉ is N, Z₁₀ is K, Z₁₁ is absent, and Z₁₂ is absent, and
   in a fourth preferred embodiment
d) Z₁ is absent, Z₂ is absent, Z₃ is absent, Z₄ is B, Z₅ is B, Z₆ is S, Z₇ is S, Z₈ is S, Z₉ is N, Z₁₀ is absent, Z₁₁ is absent, and Z₁₂ is absent, or
e) Z₁ is absent, Z₂ is absent, Z₃ is absent, Z₄ is absent, Z₅ is B, Z₆ is S, Z₇ is S, Z₈ is S, Z₉ is N, Z₁₀ is absent, Z₁₁ is absent, and Z₁₂ is absent, or
f) Z₁ is absent, Z₂ is absent, Z₃ is absent, Z₄ is B, Z₅ is B, Z₆ is S, Z₇ is S, Z₈ is S, Z₉ is absent, Z₁₀ is absent, Z₁₁ is absent, and Z₁₂ is absent, and
   in a fifth preferred embodiment
d) Z₁ is absent, Z₂ is absent, Z₃ is absent, Z₄ is absent, Z₅ is B, Z₆ is S, Z₇ is S, Z₈ is V, is absent, Z₁₀ is absent, Z₁₁ is absent, and Z₁₂ is absent, or
e) Z₁ is absent, Z₂ is absent, Z₃ is absent, Z₄ is absent, Z₅ is B, Z₆ is S, Z₇ is S, Z₈ is absent, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is absent, Z₆ is S, Z₇ is S, Z₈ is V, Z₉ is absent, Z₁₀ is absent, Z₁₁ is absent, Z₁₂ is absent, and Z₁₃ is absent, and
   in a sixth preferred embodiment
d) Z₁ is absent, Z₂ is absent, Z₃ is absent, Z₄ is absent, is absent, Z₆ is S, Z₇ is S, Z₈ is absent, is absent, Z₁₀ is absent, Z₁₁ is absent, and Z₁₂ is absent, or
e) Z₁ is absent, Z₂ is absent, Z₃ is absent, Z₄ is absent, Z₅ is absent, Z₆ is absent, Z₇ is S, Z₈ is absent, Z₉ is absent, Z₁₀ is absent, Z₁₁ is absent, and Z₁₂ is absent, or
f) Z₁ is absent, Z₂ is absent, Z₃ is absent, Z₄ is absent, Z₅ is absent, Z₆ is S, Z₇ is absent, Z₈ is absent, Z₉ is absent, Z₁₀ is absent, Z₁₁ is absent, Z₁₂ is absent, and Z₁₃ is absent, and
   in a seventh preferred embodiment
Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent.

The first terminal stretch of nucleotides of glucagon binding nucleic acid molecule 259-F5-001 and 59-E7 comprises a nucleotide sequence of 5′ Z₁Z₂Z₃Z₄Z₅Z₆GAT 3′ and the second terminal stretch of nucleotides glucagon binding nucleic acid molecule 259-F5-001 comprises a nucleotide sequence of 5′ CGAZ₇Z₈Z₉Z₁₀Z₁₁Z₁₂ 3′, wherein Z₁ is C, Z₂ is G, Z₃ is A, Z₄ is G, Z₅ is T, Z₆ is C, Z₇ is C, Z₈ is G, Z₉ is A, Z₁₀ is G, Z₁₁ is A, and Z₁₂ is C. Moreover at the 3′-end of the second terminal stretch of nucleotides there is an additional ‘G’.

Combining the first terminal stretches of nucleotides and the second terminal stretches of nucleotides of glucagon binding nucleic acid molecules 259-D5-001, 259-H6-001, 259-B7-001, 259-B8-001, 259-A5-001, 259-C8-001 and 259-E5-001 the generic formula for the first terminal stretch of nucleotides is 5′ Z₁Z₂Z₃Z₄Z₅Z₆GAG 3′ and the generic formula for the second terminal stretch of nucleotides is 5′ CTCZ₇Z₈Z₉Z₁₀Z₁₁Z₁₂ 3′, wherein
d) Z₁ is C, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is G, or
e) Z₁ is absent, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is G, or
f) Z₁ is C, Z₂ is G, Z₃ is R, Z₄ is C, Z₅ is T, Z₆ is C, Z₇ is G, Z₈ is A, Z₉ is G, Z₁₀ is T, Z₁₁ is C, and Z₁₂ is absent,
   wherein the glucagon binding nucleic acids with the best binding affinity to glucagon comprise the following combinations of the first terminal stretch and the second terminal stretch of nucleotides:
   259-H6-001: 5′ CGACTCGAG 3′ (first terminal stretch of nucleotides) and 5′ CTCGAGTCG 3′ (second terminal stretch of nucleotides);
   259-C8-0015′ CGGCTCGAG 3′ (first terminal stretch of nucleotides) and 5′ CTCGAGTCG 3′ (second terminal stretch of nucleotides).

Glucagon binding nucleic acid molecules 259-H6-002, 259-H6-006, 259-H6-007, 259-H6-008, 259-H6-002-R13, 259-H6-002-R24, 259-H6-002-R36, 259-H6-002-R13/24, 259-H6-002-R13/36, 259-H6-002-R24/36 and 259-H6-002-R13/24/36 comprise a first terminal stretch of nucleotides with a sequence of 5′ Z₁Z₂Z₃Z₄Z₅Z₆GAG 3′ and a second terminal stretch of nucleotides with a sequence of 5′ CTCZ₇Z₈Z₉Z₁₀Z₁₁Z₁₂ 3′, wherein
a) Z₁ is absent, Z₂ is absent, Z₃ is A, Z₄ is C, Z₅ is T, Z₆ is C, Z₇ is G, Z₈ is A, Z₉ is G, Z₁₀ is T, Z₁₁ is absent, and Z₁₂ is absent, or
b) Z₁ is absent, Z₂ is absent, Z₃ is A, Z₄ is C, Z₅ is T, Z₆ is C, Z₇ is G, Z₈ is A, Z₉ is G, Z₁₀ is absent, Z₁₁ is absent, and Z₁₂ is absent, or
c) Z₁ is absent, Z₂ is absent, Z₃ is absent, Z₄ is C, Z₅ is T, Z₆ is C, Z₇ is G, Z₈ is A, Z₉ is G, Z₁₀ is T, Z₁₁ is absent, and Z₁₂ is absent.

Combining the first terminal stretches of nucleotides and the second terminal stretches. of nucleotides of glucagon binding nucleic 259-H6-005, 259-H6-005-R12, 259-H6-009-R12, 259-H6-010-R12, 259-H6-011-R12, 259-H6-012-R12, 259-H6-013-R12, 259-H6-014-R12, 259-H6-015-R12, 259-H6-016-R12, 259-H6-014-R12/23/35 and 259-H6-014-R12/23/29/35/38, the generic formula for the first terminal stretch of nucleotides is 5′ Z₁Z₂Z₃Z₄Z₅Z₆SAG 3′ and the generic formula for the second terminal stretch of nucleotides is 5′ CTSZ₇Z₈Z₉Z₁₀Z₁₁Z₁₂ 3′,

wherein
a) Z₁ is absent, Z₂ is absent, Z₃ is absent, Z₄ is B, Z₅ is B, Z₆ is S, Z₇ is S, Z₈ is S, Z₉ is V, Z₁₀ is absent, Z₁₁ is absent, and Z₁₂ is absent, or
b) Z₁ is absent, Z₂ is absent, Z₃ is absent, Z₄ is absent, Z₅ is B, Z₆ is S, Z₇ is S, Z₈ is S, Z₉ is V, Z₁₀ is absent, Z₁₁ is absent, and Z₁₂ is absent, or
c) Z₁ is absent, Z₂ is absent, Z₃ is absent, Z₄ is B, Z₅ is B, Z₆ is S, Z₇ is S, Z₈ is S, Z₉ is absent, Z₁₀ is absent, Z₁₁ is absent, and Z₁₂ is absent,
   wherein the glucagon binding nucleic acids with the best binding affinity to glucagon comprise the following combinations of the first terminal stretch and the second terminal stretch of nucleotides:
c) 259-H6-005-R12: 5′ GTCGAG 3′ (first terminal stretch of nucleotides) and 5′ CTCGAC 3′ (second terminal stretch of nucleotides), or
d) 259-H6-010-R12: 5′ TGCGAG 3′ (first terminal stretch of nucleotides) and 5′ CTCGCA 3′ (second terminal stretch of nucleotides), or
e) 259-H6-012-R12: 5′ GGCCAG 3′ (first terminal stretch of nucleotides) and 5′ CTGGCC 3′ (second terminal stretch of nucleotides), or
f) 259-H6-014-R12: 5′ GCCGAG 3′ (first terminal stretch of nucleotides) and 5′ CTCGGC 3′ (second terminal stretch of nucleotides), or
g) 259-H6-015-R12: 5′ CTCGAG 3′ (first terminal stretch of nucleotides) and 5′ CTCGAG 3′ (second terminal stretch of nucleotides).

The first terminal stretch of nucleotides of glucagon binding nucleic acid molecule 259-H6-003 comprises a nucleotide sequence of 5′ Z₁Z₂Z₃Z₄Z₅Z₆GAG 3′ and the second terminal stretch of nucleotides glucagon binding nucleic acid molecule 259-H6-003 comprises a nucleotide sequence of 5′ CTCZ₇Z₈Z₉Z₁₀Z₁₁Z₁₂ 3′, wherein
a) Z₁ is absent, Z₂ is absent, Z₃ is absent, Z₄ is absent, Z₅ is T, Z₆ is C, Z₇ is G, Z₈ is A, Z₉ is absent, Z₁₀ is absent, Z₁₁ is absent, and Z₁₂ is absent, or
b) Z₁ is absent, Z₂ is absent, Z₃ is absent, Z₄ is absent, Z₅ is T, Z₆ is C, Z₇ is G, Z₈ is absent, Z₉ is absent, Z₁₀ is absent, Z₁₁ is absent, and Z₁₂ is absent, or
c) Z₁ is absent, Z₂ is absent, Z₃ is absent, Z₄ is absent, Z₅ is absent, Z₆ is C, Z₇ is G, Z₈ is A, Z₉ is absent, Z₁₀ is absent, Z₁₁ is absent, and Z₁₂ is absent, preferably
   the first terminal stretch of nucleotides is 5′-TCGAG-3 and the second terminal stretch of nucleotides is 5′-CTCGA-3.

The first terminal stretch of nucleotides of glucagon binding nucleic acid molecule 259-H6-004 comprises a nucleotide sequence of 5′ Z₁Z₂Z₃Z₄Z₅Z₆GAG 3′ and the second terminal stretch of nucleotides glucagon binding nucleic acid molecule 259-H6-004 comprises a nucleotide sequence of 5′ CTCZ₇Z₈Z₉Z₁₀Z₁₁Z₁₂ 3′, wherein Z₁ is absent, Z₂ is absent, Z₃ is absent, Z₄ is absent, Z₅ is absent, Z₆ is absent, Z₇ is absent, Z₈ is absent, Z₉ is absent, Z₁₀ is absent, Z₁₁ is absent, and Z₁₂ is absent.

In order to determine the binding affinity by surface plasmon resonance measurement and/or to prove the functionality of glucagon binding nucleic acid molecules of Type B, molecules 259-H6-002, 259-H6-002-R13, 259-H6-002-R24, 259-H6-002-R36, 259-H6-002-R13/24, 259-H6-002-R13/36, 259-H6-002-R13/24/36, 259-H6-002-R24/36, 259H6-014-R12, 259-H6-014-R12/23/35 and 259-H6-014-R12/23/29/35/38 were synthesized as spiegelmers, whereby spiegelmers 259-H6-002, 259-H6-002-R13 and 259-H6-014-R12/23/35 were synthesized with an amino-group at the 5′-end. To the amino-modified spiegelmers 259-259-H6-002-5′-Amino [SEQ ID NO: 155], H6-002-R13-5′-amino [SEQ ID NO: 156] and 259-H6-014-R12/23/35-5′-amino [SEQ ID NO: 157] a 40 kDa PEG-moiety was coupled leading to glucagon binding spiegelmers 259-H6-002-5′-PEG (also referred to as NOX-G12) [SEQ ID NO: 88], 259-H6-002-R13-5′-PEG (also referred to as NOX-G13) [SEQ ID NO: 89], and 259-H6-014-R12/23/35-5′-PEG (also referred to as NOX-G14) [SEQ ID NO: 90], Synthesis and PEGylation of the spiegelmer is described in Example 2.

The equilibrium binding constants K_{D} of glucagon binding spiegelmers 259-H6-002, 259-H6-002-R13, 259-H6-002-R24, 259-H6-002-R36, 259-H6-002-R13/24, 259-H6-002-R13/36, 259-H6-002-R13/24/36, 259-H6-002-R24/36, 259-H6-014-R12, 259-H6-014-R12/23/35, NOX-G13 and NOX-G14 were determined by surface plasmon resonance measurement (FIG. 6C, 259-H6-014-R12/23/29/35/38,10, 11, 12, 13, protocol see Example 4).

Glucagon binding spiegelmers NOX-G13 and NOX-G14 were able to inhibit/antagonize in vitro the function of glucagon to its receptor with an IC₅₀ of 4.7-6.0 nM (FIG. 20 A; for protocol of the in vitro functional assay see Example 5).

The data of the surface plasmon resonance measurement as shown in FIG. 10 confirm that replacing one 2′deoxyribonucleotide by one ribonucleotide in the central stretch of nucleotides of glucagon binding molecule 259-H6-002 led to an improved binding affinity (shown for 259-H6-002-R13, 259-H6-002-R24, 259-H6-002-R36). The data of the surface plasmon resonance measurement as shown in FIG. 12 reveal that replacing additional one or two 2′deoxyribonucleotides by one or two ribonucleotides in the central stretch glucagon binding molecule 259-H6-002R13 lead to a further improved binding affinity to glucagon (shown for 259-H6-002-R13, 259-H6-002-R13_R24, 259-H6-002-R13_R36 and 259-H6-002-R13_R24_R36). This effect was also shown for spiegelmers 259-H6-002, 259-H6-002-R13 and 259-H6-002-R13-R24-R36 in an in vitro functional assay (FIG. 16, for protocol see Example 5).

Furthermore, as shown in example 6 the binding selectivity of the glucagon binding spiegelmers NOX-G13 and NOX-G14 was determined (FIGS. 19 and 20).

### 1.3 Glucagon Binding Nucleic Acid Molecules of Type C

Additionally, further five glucagon binding nucleic acids that do not share the glucagon binding motifs of ‘Type A’ and ‘Type B’ were identified and are referred to herein as “type C”. They were analyzed as aptamers using the direct pull-down binding assay and or comparative competition pull-down binding assay (FIGS. 7 and 8).

The inventors surprisingly showed by plasmon resonance measurement that the binding affinity of glucagon binding nucleic acid molecule NOX-G11stabi2 was improved by replacing one ribonucleotide by 2′-deoxyribonucleotide in the sequence of NOX-G11stabi2. The 2′-deoxyribonucleotides and ribonucleotides are shown in FIGS. 29 and 30A-B, wherein in Example 1.3 and in the corresponding figures the following abbreviations were used: G is guanosine(5′monophosphate), C is cytidine 5′monophosphate, A is adenosine(5′monophosphate), U is uridine(5′monophosphate), dG is 2′deoxy-guanosine(5′monophosphate), dC is 2′deoxy-cytidine(5′monophosphate), dA is 2′deoxy-adenosine(5′ monophosphate), dT is 2′deoxy-thymidine(5′monophosphate). In particular replacing one ribonucleotide by 2′-deoxyribonucleotide at position 5, 7, 15, 16, 19, 20, 21, 22, 23, 24, 25, 26, 27, 46 or 48 in the glucagon binding nucleic acid molecule NOX-G11stabi2 led to improved binding to glucagon (FIGS. 25A and 25B). In FIG. 26 the binding curves of NOX-G11stabi2, NOX-G11-D07, NOX-G11-D16, NOX-G11-D19, NOX-G11-D21 and NOX-G 11-D22 as determined by plasmon resonance measurement are shown.

It is to be understood that any of the sequences shown in FIGS. 1 through 8 are nucleic acid molecules according to the present invention, including those truncated forms thereof but also including those extended forms thereof under the proviso, however, that the thus truncated and extended, respectively, nucleic acid molecules are still capable of binding to the target.

### EXAMPLE 2

### Synthesis and Derivatization of Aptamers and Spiegelmers

### Small Scale Synthesis

The nucleic acid molecules of the present invention were produced as aptamers (D-RNA nucleic acids or D-DNA modified D-RNA nucleic acids) and spiegelmers (L-RNA nucleic acids or L-DNA modified L-RNA nucleic acids), respectively, by solid-phase synthesis with an ABI 394 synthesizer (Applied Biosystems, Foster City, Calif., USA) using 2′TBDMS RNA and DNA phosphoramidite chemistry with standard exocyclic amine protecting groups (Damha and Ogilvie, 1993). For the RNA part of the oligonucleotide rA(N-Bz)-, rC(N—Ac)-, rG(N-ibu)-, and rU-phosphoramidites in the D- and L-configuration were used, while for the DNA part dA(N-Bz)-, dC(N—Ac)-, dG(N-ibu)-, and dT in the D- and L-configuration were applied. All phosphoramidites were purchased from ChemGenes, Wilmington, Mass. After synthesis and deprotection aptamers and spiegelmers were purified by gel electrophoresis.

### Large Scale Synthesis Plus Modification

Spiegelmers were produced by solid-phase synthesis with an ÄktaPilot100 synthesizer (GE Healthcare, Freiburg) using 2′TBDMS RNA and DNA phosphoramidite chemistry with standard exocyclic amine protecting groups (Damha and Ogilvie, 1993). L-rA(N-Bz)-, L-rC(N—Ac)-, L-rG(N-ibu)-, L-rU-, L-dA(N-Bz)-, L-dC(N—Ac)-, L-dG(N-ibu)-, and L-dT-phosphoramidites were purchased from ChemGenes, Wilmington, Mass. The 5′-amino-modifier was purchased from American International Chemicals Inc. (Framingham, Mass., USA). Synthesis of the unmodified or a 5′-Amino-modified spiegelmer was started on L-riboA, L-riboC, L-riboG, L-riboU, L-2′deoxyA, L-2′deoxyC, L-2′deoxyG, or L-2′deoxyT modified CPG pore size 1000 Å (Link Technology, Glasgow, UK. For coupling of the RNA and DNA phosphoramidites (15 min per cycle), 0.3 M benzylthiotetrazole (CMS-Chemicals, Abingdon, UK) in acetonitrile, and 2 equivalents of the respective 0.2 M phosphoramidite solution in acetonitrile was used. An oxidation-capping cycle was used. Further standard solvents and reagents for oligonucleotide synthesis were purchased from Biosolve (Valkenswaard, NL). The Spiegelmer was synthesized DMT-ON; after deprotection, it was purified via preparative RP-HPLC (Wincott et al., 1995) using Source15RPC medium (Amersham). The 5′DMT-group was removed with 80% acetic acid (30 min at RT). In case of 5′amino modified Spiegelmers the 5′MMT-group was removed with 80% acetic acid (90 min at RT). Subsequently, aqueous 2 M NaOAc solution was added and the Spiegelmer was desalted by tangential-flow filtration using a 5 K regenerated cellulose membrane (Millipore, Bedford, Mass.).

### Pegylation of Spiegelmers

In order to prolong the Spiegelmer's plasma residence time in vivo, a 40 kDa polyethylene glycol (PEG) moiety was covalently coupled at the 5′-end of the spiegelmers.

For PEGylation (for technical details of the method for PEGylation see European patent application EP 1 306 382), the purified 5′-amino modified Spiegelmer was dissolved in a mixture of H₂O (2.5 ml), DMF (5 ml), and buffer A (5 ml; prepared by mixing citric acid•H₂O [7 g], boric acid [3.54 g], phosphoric acid [2.26 ml], and 1 M NaOH [343 ml] and adding water to a final volume of 1 l; pH=8.4 was adjusted with 1 M HCl).

The pH of the Spiegelmer solution was brought to 8.4 with 1 M NaOH. Then, 40 kDa PEG-NHS ester (Jenkem Technology, Allen, Tex., USA) was added at 37° C. every 30 min in six portions of 0.25 equivalents until a maximal yield of 75 to 85% was reached. The pH of the reaction mixture was kept at 8-8.5 with 1 M NaOH during addition of the PEG-NHS ester.

The reaction mixture was blended with 4 ml urea solution (8 M), and 4 ml buffer B (0.1 M triethylammonium acetate in H₂O) and heated to 95° C. for 15 min. The PEGylated Spiegelmer was then purified by RP-HPLC with Source 15RPC medium (Amersham), using an acetonitrile gradient (buffer B; buffer C: 0.1 M triethylammonium acetate in acetonitrile). Excess PEG eluted at 5% buffer C, PEGylated Spiegelmer at 10-15% buffer C. Product fractions with a purity of >95% (as assessed by HPLC) were combined and mixed with 40 ml 3 M NaOAC. The PEGylated Spiegelmer was desalted by tangential-flow filtration (5 K regenerated cellulose membrane, Millipore, Bedford Mass.).

### EXAMPLE 3

### Determination of Binding Affinity to Glucagon (Pull-Down Assay)

For binding analysis to glucagon the glucagon binding nucleic acid molecules were synthesized as aptamers consisting of D-nucleotides or as spiegelmers consisting of L-nucleotides. The binding analysis of aptamers was done with biotinylated human D-glucagon consisting of D-amino acids. The binding analysis of spieglmers was done with biotinylated human L-glucagon consisting of L-amino acids.

### Direct Pull-Down Assay

Aptamers were 5′-phosphate labeled by T4 polynucleotide kinase (Invitrogen, Karlsruhe, Germany) using [γ-³²P]-labeled ATP (Hartmann Analytic, Braunschweig, Germany). Two additional adenosin residues in the D-configuration at the Spiegelmer's 5′-end enabled also the radioactive labeling of spiegelmers by T4 polynucleotide kinase. The specific radioactivity of labeled nucleic acids was 200,000-800,000 cpm/pmol. After de- and renaturation (1′ 94° C., ice/H₂O) labeled nucleic acids were incubated at 100-700 pM concentration at 37° C. in selection buffer (20 mM Tris-HCl pH 7.4; 137 mM NaCl; 5 mM KCl; 1 mM MgCl₂; 1 mM CaCl₂; 0.1% [w/vol] Tween-20; 0.1% [w/vol] CHAPS) together with varying amounts of biotinylated human D- or L-glucagon, respectively, for 2-6 hours in order to reach equilibrium at low concentrations. Selection buffer was supplemented with 100 g/ml human serum albumin (Sigma-Aldrich, Steinheim, Germany), and 10 g/ml yeast RNA (Ambion, Austin, USA) in order to prevent unspecific adsorption of binding partners to surfaces of used plasticware or to the immobilization matrix. The concentration range of biotinylated D-glucagon for aptamer binding was set from 0.64 nM to 10 μM whereas the concentration range of biotinylated L-glucagon for Spiegelmer binding was set from 0.32 nM to 5 μM; total reaction volume was 50 μl. Biotinylated glucagon and complexes of nucleic acids and biotinylated glucagon were immobilized on 4 μl High Capacity Neutravidin Agarose particles (Thermo Scientific, Rockford, USA) which had been preequilibrated with selection buffer. Particles were kept in suspension for 20 min at the respective temperature in a thermomixer. Immobilized radioactivity was quantitated in a scintillation counter after removal the supernatant and appropriate washing. The percentage of binding was plotted against the concentration of biotinylated glucagon and dissociation constants were obtained by using software algorithms (GRAFIT; Erithacus Software; Surrey U.K.) assuming a 1:1 stoichiometry.

### Competitive Pull-Down Assay for Ranking of Glucagon Binding Nucleic Acids

In order to compare the binding of different aptamers or Spiegelmers to glucagon a competitive ranking assay was performed. For this purpose either the most affine aptamer spiegelmer available was radioactively labeled (see above) and served as reference for glucagon binding aptamers or spiegelmers, respectively. After de- and renaturation the labeled nucleic acids were incubated at 37° C. with biotinylated glucagon in 50 or 100 μl selection buffer at conditions that resulted in around 5-10% binding to the biotinylated glucagon after immobilization on 1.5 μl High Capacity Neutravidin Agarose particles (Thermo Scientific, Rockford, USA) and washing without competition. An excess of de- and renatured non-labeled aptamer variants was added at different concentrations (e.g. 50, 500, and 5000 nM) together with the labeled reference aptamer to parallel binding reactions. De- and renatured non-labeled Spiegelmer derivatives were applied at concentrations of 1, 10, and 100 nM together with the reference Spiegelmer in parallel binding reactions. The nucleic acids to be tested competed with the reference nucleic acid for target binding, thus decreasing the binding signal in dependence of their binding characteristics. The aptamer or Spiegelmer, respectively that was found most active in this assay could then serve as a new reference for comparative analysis of other glucagon binding nucleic acid molecules. The binding of labeled Spiegelmer of each binding curve was normalized setting the binding without competition to 100%.

### Competitive Pull-Down Assay for Determination of Affinity and Selectivity

In addition to comparative ranking experiments the competitive pull-down assay was also performed to determine the affinity constants of glucagon binding nucleic acids. For this purpose either a D-glucagon binding aptamer or a L-glucagon binding Spiegelmer was radioactively labeled and served as reference as described above. After de- and renaturation the labeled reference nucleic acid and a set of 5-fold dilutions ranging e.g. from 0.128 to 2000 nM of competitor molecules were incubated with a constant amount of biotinylated glucagon in 0.1 or 0.2 ml selection buffer at 37° C. for 2-4 hours. The chosen protein concentration should cause final binding of approximately 5-10% of the radiolabeled reference molecule at the lowest competitor concentration. In order to measure the binding constants of derivative nucleic acid sequences an excess of the appropriate de- and renatured non-labeled aptamer or Spiegelmer variants served as competitors, whereas for Spiegelmers unmodified as well as PEGylated forms were tested. In another assay approach non-biotinylated glucagon at different concentrations competed against the biotinylated glucagon for aptamer or Spiegelmer binding. Furthermore, the selectivity of the glucagon binding Spiegelmers was investigated by human glucagon-like peptide-1 (GLP-1) and glucose-dependent insulinotropic polypeptide (GIP) which were used to compete against the biotinylated glucagon. After immobilization of biotinylated glucagon and the bound nucleic acids on 1.5 μl High Capacity Neutravidin Agarose matrix, washing and scintillation counting (see above), the normalized percentage of bound radiolabeled Spiegelmer was plotted against the corresponding concentration of competitor molecules. The resulting dissociation constant was calculated employing the GraFit Software.

### EXAMPLE 4

### Biacore measurement of glucagon-binding spiegelmers

### Biacore Assay Setup

Biotinylated human L-glucagon (glucagon₁₋₂₉-AEEAc-AEEAc-biotin, custom synthesis by BACHEM, Switzerland) was immobilized on a carboxymethylated (abbr. CM) dextran-coated sensor chip which had been prepared by covalent immobilization of soluble neutravidin (Sigma Aldrich, Germany) using a 1:1 mixture of 0.4 M EDC (1-ethyl-3-(3-dimethylaminopropyl) carbodiimide in H₂O; GE, BR-1000-50) and 0.1M NHS (N-hydroxysuccinimide in H₂O; GE, BR-1000-50). The reference flow cell on the same sensor chip was blocked with biotin.

### General Kinetic Evaluation

The glucagon binding Spiegelmers were dissolved in water to a stock concentration of 100 μM (quantification by UV measurement), heated up to 95° C. for 30 seconds in a water bath or thermo mixer and snap cooled on ice to assure a homogenous dissolved solution. Kinetic parameters and dissociation constants were evaluated by a series of Spiegelmer injections at concentrations of 1000, 500, 250, 125, 62.5, 31.25, 15.63, 7.8, 3.9, 1.95, 0.98 and 0 nM diluted in running buffer. In all experiments, the analysis was performed at 37° C. using the Kinject command defining an association time of 240 to 360 and a dissociation time of 240 to 360 seconds at a flow of 30 μl/min. The assay was double referenced, whereas FC1 served as (blocked) surface control (bulk contribution of each Spiegelmer concentration) and a series of buffer injections without analyte determined the bulk contribution of the buffer itself. Data analysis and calculation of dissociation constants (K_{D}) was done with the BIAevaluation 3.1.1 software (BIACORE AB, Uppsala, Sweden) using a modified Langmuir 1:1 stoichiometric fitting algorithm.

Data analysis and calculation of dissociation constants (KD) was done with the BIAevaluation 3.1.1 software (BIACORE AB, Uppsala, Sweden) using a modified Langmuir 1:1 stoichiometric fitting algorithm, with a constant RI and mass transfer evaluation with a mass transport coefficient kt of 1×107 [RU/M*s]. The results were plotted as ka [l/M*s] versus kd [l/s].

### Competitive Biacore Assay to Determine the Selectivity of Glucagon-Binding Spiegelmers

Immobilization of biotinylated human glucagon was performed as described above. The Spiegelmer to be analysed was injected at a fixed concentration (here 125 nM) together with a concentration series (2000-1000-500-250-0 nM) of various glucagon related free peptides (namely glucagon, oxytomodulin, GLP-1 (7-37), GLP-2(1-33), GIP and Prepro-VIP (81-122) as competitor or no competitor as control. Spiegelmer binding to immobilized L-glucagon without competitor (control) was normalized to 100%. When the Spiegelmer is co-injected with glucagon or related peptides (competitor peptides), Spiegelmer association to immobilized glucagon is reduced if binding to the soluble competitor occurs (responses shown only for 2000 nM of competitor peptides). The response units [RU] after 360 seconds of injection were determined, normalized to the control (=100%) and plotted.

### EXAMPLE 5

### Inhibition of Glucagon-Induced cAMP Production by Glucagon-Binding Spiegelmers

A stably transfected cell line expressing the human receptor for glucagon was generated by cloning the sequence coding for the human glucagon receptor (NCBI accession NM_{—}000160) into the pCR3.1 vector (Invitrogen). CHO cells adapted to growth in serum-free medium (UltraCHO, Lonza) were transfected with the glucagon receptor plasmid and stably transfected cells were selected by treatment with geneticin.

For an inhibition experiment CHO cells expressing the glucagon receptor were plated on a 96 well plate (cell culture treated, flat bottom) at a density of 4-6×10⁴/well and cultivated overnight at 37° C. 5% CO₂ in UltraCHO medium containing 100 units/ml penicillin, 100 μg/ml streptomycin and 0.5 mg/ml geneticin. 20 min before stimulation a solution of 3-isobutyl-1-methylxanthine (IBMX) was added to a final concentration of 1 mM. The stimulation solutions (glucagon+various concentrations of Spiegelmers) were made up in Hank's balanced salt solution (HBSS)+1 mg/ml BSA and were incubated for 30 min at 37° C. Shortly before addition to the cells, IBMX was added to a final concentration of 1 mM. For stimulation, the medium was removed from the cells and the stimulation solutions (glucagon+Spiegelmer) were added. After incubation for 30 min at 37° C. the solutions were removed and the cells were lysed in lysis-buffer which is a component of the cAMP-Screen™ System kit (Applied Biosystems). This kit was used for determination of the cAMP content following the supplier's instructions.

### EXAMPLE 6

### Inhibition of GIP-Induced cAMP Production by Glucagon-Binding Spiegelmers

To investigate whether glucagon-binding Spiegelmers can also block the action of glucagon-dependent insulinotropic polypeptide (GIP), RIN-m5F rat insuloma cells (ATCC; CRL-11605) were plated on a 96 well plate (cell culture treated, flat bottom) at a density of 1×10⁵/well and cultivated overnight at 37° C. 5% CO₂ in RPMI1640 medium containing 10% fetal bovine serum, 100 units/ml penicillin and 100 μg/ml streptomycin. 20 min before stimulation a solution of 3-isobutyl-1-methylxanthine (IBMX) was added to a final concentration of 1 mM.

The stimulation solutions (GIP+various concentrations of Spiegelmers) were made up in Hank's balanced salt solution (HBSS)+1 mg/ml BSA and were incubated for 30 min at 37° C. Shortly before addition to the cells, IBMX was added to a final concentration of 1 mM. For stimulation, the medium was removed from the cells and the stimulation solutions (GIP+Spiegelmer) were added. After incubation for 30 min at 37° C. the solutions were removed and the cells were lysed in lysis-buffer which is a component of the cAMP-Screen™ System kit (Applied Biosystems). This kit was used for determination of the cAMP content following the supplier's instructions.

### EXAMPLE 7

### Determination of Glucagon Binding Spiegelmer Selectivity

The glucagon precursor is cleaved into 8 chains, namely Glicentin, Glicentin-related polypeptide, (GRPP), oxyntomodulin (OXY/OXM), glucagon, glucagon-like peptide 1 (GLP-1), glucagon-like peptide 1(GLP-1[7-37]), Glucagon-like peptide 1 (GLP-1[7-36]) and glucagon-like peptide 2 (GLP-2) (see FIG. 21). A BLAST-search also identified glucose-dependent insolinotropic peptide (GIP) and intestinal peptide PHV-42 (Prepro-vasoactive intestinal peptide/Prepro-VIP [81-122]) as glucagon sequence related peptides. Selectivity of glucagon binding nucleic acid molecules of Type A—such as Spiegelmers 257-E1-6xR-001, 257-E1-7xR-037, 257-E1-6xR-030-5′-PEG (also referred to as NOX-G15) and 257-E1-7xR-037-5′-PEG (also referred to as NOX-G16) and of Type B—such as 259-H6-002-R13-5′-PEG (also referred to as NOX-G13) and 259-H6-014-R12/23/35-5′-PEG (also referred to as NOX-G14)—was determined in a competitive binding assay format with free glucagon, oxyntomodulin, GLP-1 [7-37], GLP-2 [1-33], GIP and Prepro-VIP[81-122] by pull-down assays (see Example 3) and/or Biacore measurement (see Example 4). Cell-based assays (Example 5 and 6) were used to confirm binding glucagon binding nucleic acid molecules of Type A to glucagon, oxyntomodulin, GLP-1 and GIP.

In the pull-down assays (see Example 3) and/or Biacore measurements glucagon binding nucleic acid molecules of Type A and Type B showed comparable binding to glucagon and oxyntomodulin and inhibited glucagon-induced, as well as oxyntomodulin-induced cAMP formation in cell-based assays. These data indicate that the C-terminus of glucagon is not essential for glucagon binding of the glucagon binding nucleic acid molecules of Type A and Type B. The glucagon sequence-related peptides GLP-1 [7-37], GLP-2 [1-33] and Prepro-VIP [81-122] were not recognized by glucagon binding nucleic acid molecules of Type A and Type B. Surprisingly the glucagon binding nucleic acid molecules of Type B 259-H6-002-R13-5′-PEG (also referred to as NOX-G13) and 259-H6-014-R12/23/35-5′-PEG (also referred to as NOX-G14) showed binding to GIP and inhibited GIP induced cAMP formation in cell-based assays (FIGS. 18, 19, 20).

### EXAMPLE 8

### Effect of Glucagon Binding Spiegelmers on Glucose Tolerance in a Type 1 and a Type 2 Diabetes Mellitus Animal Experiment

### 8.1 Effect of Glucagon Binding Spiegelmer NOX-G15 on Glucose Tolerance in a Type 1 Diabetes Mellitus Animal Experiment

### Methods

Male BALB/c mice were obtained at 20-24 g and housed in standard conditions for one week before starting the experiment.

According to recently published data (Lee, Wang et al. 2011), type 1 diabetes mellitus (abbr. DM1) was induced by a first streptozotocin (abbr. STZ) injection (100 mg/kg body weight) three weeks prior to the test day and a second injection (80 mg/kg body weight) two weeks before the experiment. In order to verify the type 1 diabetes phenotype achievement fasting glucose levels and body weight were measured one day before the intraperitoneal glucose tolerance test (abbr. ipGTT). Animals with a weight loss >25% as compared with the initial body weight and animals with fasting blood glucose levels below 200 mg/dL or above 500 mg/dL were excluded.

On the experimental day the following procedures were done:

The mice were fasted for 2.5 h before the beginning (time: −95 min) of the experiment.

| Time: | | Action |
|---|---|---|
| −95 | min: | determination of basal plasma glucose |
| −90 | min: | i.p. injection of NOX-G15 |
| | | (1 mg/kg and 10 mg/kg) or the glucagon receptor |
| | | antagonist des-His¹-Glu⁹-glucagon |
| | | (2 mg/kg and 4 mg/kg) or vehicle (H₂O for |
| | | injection). |
| −5 | min: | determination of blood glucose |
| 0 | min: | i.p. injection of glucose (2 g/kg) |
| 20 | min: | determination of blood glucose |
| 40 | min: | determination of blood glucose |
| 70 | min: | determination of blood glucose |
| 100 | min: | determination of blood glucose |

### Results

The STZ-treated mice presented with strongly elevated basal glucose level between 300 and 400 mg/dL after the 2.5 h fasting interval. 20 min after the i.p. glucose injection glucose levels peaked highest in the vehicle-treated group. The peptidic receptor antagonist that was used as positive control (Dallas-Yang, Shen et al., 2004) showed a drop in the glucose concentration in the high dose group before the glucose challenge. Both groups peaked lower than the vehicle group. Both Spiegelmer dose groups also had a lower peak glucose concentration than vehicle. The effects described above also resulted in a significantly lower area under the curve for blood glucose over time in the groups treated with Spiegelmer (abbr. AUC) (FIG. 23).

### 8.2 Effect of Glucagon Binding Spiegelmer NOX-G15 on Glucose Tolerance in a Type 2 Diabetes Mellitus Animal Experiment

To mimic late-stage type 2 diabetes mellitus (abbr. DM2) symptoms observed in humans, diet-induced obese mice can be treated with low doses of streptozotocin (Luo, Quan et al. 1998; Strowski, Li et al. 2004).

### Methods

Male BALB/c mice were obtained at 20-24 g. Insulin resistance was induced 10 weeks of high-fat diet (abbr. HFD) feeding. Additionally after 8 weeks of HFD a dose of STZ (100 mg/kg body weight) was administered to induce partial β-cell failure which mimics late-stage DM2 physiology (Baribault 2010). Diabetes was confirmed by measuring fasting blood glucose levels and body weight. Mice with blood glucose below 200 mg/dL or above 300 mg/dL were excluded. Likewise, mice that did not have a stable weight profile before and 1 week after the streptozotocin injection in spite of the HFD were excluded.

On the experimental day the following procedures were done:

The mice were fasted for 2.5 h before the beginning (time: −120 min) of the experiment.

| Time: | | Action |
|---|---|---|
| −120 | min: | determination of basal blood glucose |
| −90 | min | i.p. injection of NOX-G15 (1 mg/kg and 10 mg/kg) |
| | | or the glucagon receptor |
| | | antagonist des-His¹-Glu⁹-glucagon |
| | | (4 mg/kg) or vehicle (H₂O for injection). |
| −5 | min: | determination of blood glucose |
| 0 | min: | i.p. injection of glucose (2 g/kg) |
| 20 | min: | determination of blood glucose |
| 40 | min: | determination of blood glucose |
| 70 | min: | determination of blood glucose |
| 100 | min: | determination of blood glucose |
| 120 | min: | determination of blood glucose |

### Results

The DM2 mice presented with elevated basal glucose level of 170 mg/dL after the 2.5 h fasting interval. 40 min after the i.p. glucose injection glucose levels peaked highest in the vehicle-treated group. The peptidic receptor antagonist used as a positive control (Dallas-Yang, Shen et al. 2004) showed a slightly lower glucose concentration and showed a faster normalization. Both Spiegelmer dose groups had a lower peak glucose concentration and a faster normalization than vehicle and the glucagon receptor antagonist. The effects described above also resulted in a significantly lower area under the curve (abbr. AUC) for blood glucose over time in the groups treated with Spiegelmer (see FIG. 24).

### 8.3 Effect of Glucagon Binding Spiegelmer NOX-G16 on Glucose Tolerance in a Type 1 Diabetes Mellitus Animal Experiment

### Methods

Male BALB/c mice were obtained at 20-24 g and housed in standard conditions for one week before starting the experiment.

According to recently published data (Lee, Wang et al. 2011), type 1 diabetes mellitus (abbr. DM1) was induced by a first streptozotocin (abbr. STZ) injection (100 mg/kg body weight) three weeks prior to the test day and a second injection (80 mg/kg body weight) two weeks before the experiment. In order to verify the type 1 diabetes phenotype achievement fasting glucose levels and body weight were measured one day before the intraperitoneal glucose tolerance test (abbr. ipGTT). Animals with a weight loss >25% as compared with the initial body weight and animals with fasting blood glucose levels below 200 mg/dL or above 500 mg/dL were excluded.

There were 20 mice per treatment group.

On the experimental day the following procedures were done:

| Time: | | Action |
|---|---|---|
| −480 | min | food removal |
| −125 | min: | determination of basal blood glucose |
| −120 | min: | i.p. injection of NOX-G16 |
| | | (0.1 mg/kg and 1 mg/kg) or vehicle (0.9% saline) |
| −5 | min: | determination of blood glucose |
| | | (effect of Spiegelmer only) |
| 0 | min: | i.p. injection of glucose (2 g/kg) |
| 15 | min: | determination of blood glucose |
| 30 | min: | determination of blood glucose |
| 45 | min: | determination of blood glucose |
| 60 | min: | determination of blood glucose |
| 90 | min: | determination of blood glucose |

Treatment was done once daily for nine days around 9 a.m.

ipGTT was done on days 1, 3 and 7

### Results

The STZ-treated mice presented with strongly elevated basal glucose level between 300 and 400 mg/dL after the 2 h fasting interval. 20 min after the i.p. glucose injection glucose levels peaked highest in the vehicle-treated group. Both Spiegelmer dose groups had a lower peak glucose concentration than vehicle. The effects described above also resulted in a significantly lower area under the curve for blood glucose over time in the groups treated with 1 mg/kg Spiegelmer (abbr. AUC) (FIG. 27). This shows that the antihyperglycemic effect of repeated doses of NOX-G16 can be maintained over seven days, showing that no overruling of the Spiegelmer effect by up- or downregulation of endocrine hormones or other signaling substances and their receptors takes place.

On day 9 NOX-G16 was administered after 4 h of fasting. After additional 2 h blood was drawn. Fibroblast growth factor 21 (FGF-21) levels (which are increased in diabetes) were significantly lowered in both Spiegelmer dose groups, thus providing evidence that repeated dosing of NOX-G16 may have a beneficial effect on the long-term outcome of the disease.

### REFERENCES

The complete bibliographic data of the documents recited herein are, if not indicated to the contrary, as follows, whereby the disclosure of said references is incorporated herein by reference.
Altschul S. F., Gish W., et al. (1990) Basic local alignment search tool. J Mol Biol. 215(3):403-10.
Altschul S. F., Madden T. L., et al. (1997) Gapped BLAST and PSI-BLAST: a new generation of protein database search programs. Nucleic Acids Res. 25(17):3389-402.
Amori, R. E., J. Lau, et al. (2007). “Efficacy and safety of incretin therapy in type 2 diabetes: systematic review and meta-analysis.” JAMA 298(2): 194-206.
Asmar, M. (2011). “New physiological effects of the incretin hormones GLP-1 and GIP.” Dan Med Bull 58(2): B4248.
Baribault, H. (2010). “Mouse models of type II diabetes mellitus in drug discovery.” Methods Mol Biol 602: 135-55.
Brand, C. L., B. Rolin, et al. (1994). “Immunoneutralization of endogenous glucagon with monoclonal glucagon antibody normalizes hyperglycaemia in moderately streptozotocin-diabetic rats.” Diabetologia 37(10): 985-93.
Conarello, S. L., G. Jiang, et al. (2007). “Glucagon receptor knockout mice are resistant to diet-induced obesity and streptozotocin-mediated beta cell loss and hyperglycaemia.” Diabetologia 50(1): 142-50.
Dallas-Yang, Q., X. Shen, et al. (2004). “Hepatic glucagon receptor binding and glucose-lowering in vivo by peptidyl and non-peptidyl glucagon receptor antagonists.” Eur J Pharmacol 501(1-3): 225-34.
Damha M. J. and Ogilvie K.K. (1993). “Oligoribonucleotide synthesis. The silyl-phosphoramidite method.” Methods Mol Biol 20:81-114
EMA (2010) “European Medicines Agency recommends suspension of Avandia, Avandamet and Avaglim; Anti-diabetes medication to be taken off the market.” Press Release Volume, 2 DOI:
FDA (2011) “FDA Drug Safety Communication: Updated Risk Evaluation and Mitigation Strategy (REMS) to Restrict Access to Rosiglitazone-containing Medicines including Avandia, Avandamet, and Avandary.” Safety Announcement Volume, DOI:
Gin, H. and V. Rigalleau (2000). “Post-prandial hyperglycemia. post-prandial hyperglycemia and diabetes.” Diabetes Metab 26(4): 265-72.
Griffing, G. T., Odeke, S., et al. “Hyperglucagonemia”, http://emedicine.medscape.com/article/121575-overview
Irwin, N. and P. R. Flatt (2009). “Evidence for beneficial effects of compromised gastric inhibitory polypeptide action, in obesity-related diabetes and possible therapeutic implications.” Diabetologia 52(9): 1724-31.
Jiang, G. and B. B. Zhang (2003). “Glucagon and regulation of glucose metabolism.”. Am J Physiol Endocrinol Metab 284(4): E671-8.
Klussmann S. (2006). The Aptamer Handbook—Functional Oligonucleotides and their Applications. Edited by S. Klussmann. WILEY-VCH, Weinheim, Germany, ISBN 3-527-31059-2
Kusser W. (2000) Chemically modified nucleic acid aptamers for in vitro selections: evolving evolution. J Biotechnol 74(1): 27-38.
Lee, Y., M. Y. Wang, et al. (2011). “Glucagon receptor knockout prevents insulin-deficient type 1 diabetes in mice.” Diabetes 60(2): 391-7.
Liang, Y., M. C. Osborne, et al. (2004). “Reduction in glucagon receptor expression by an antisense oligonucleotide ameliorates diabetic syndrome in db/db mice.” Diabetes 53(2): 410-7.
Luo, J., J. Quan, et al. (1998). “Nongenetic mouse models of non-insulin-dependent diabetes mellitus.” Metabolism 47(6): 663-8.
Mairal T., Ozalp V. C., Lozano Sanchez P., et al. (2008) Aptamers: molecular tools for analytical applications. Anal Bioanal Chem. 390(4):989-1007
McClean, P. L., N. Irwin, et al. (2007). “GIP receptor antagonism reverses obesity, insulin resistance, and associated metabolic disturbances induced in mice by prolonged consumption of high-fat diet.” Am J Physiol Endocrinol Metab 293(6): E1746-55.
McGinnis S., Madden T. L. et al. (2004) BLAST: at the core of a powerful and diverse set of sequence analysis tools. Nucleic Acids Res. 32(Web Server issue):W20-5.
Miyawaki, K., Y. Yamada, et al. (1999). “Glucose intolerance caused by a defect in the entero-insular axis: a study in gastric inhibitory polypeptide receptor knockout mice.” Proc Natl Acad Sci USA 96(26): 14843-7.
Miyawaki, K., Y. Yamada, et al. (2002). “Inhibition of gastric inhibitory polypeptide signaling prevents obesity.” Nat Med 8(7): 738-42.
Needleman and Wunsch (1970) A general method applicable to the search for similarities in the amino acid sequence of two proteins. J Mol Biol. 48(3):443-53.
Nissen, S. E. and K. Wolski (2007). “Effect of rosiglitazone on the risk of myocardial infarction and death from cardiovascular causes.” N Engl J Med 356(24): 2457-71.
Ohneda, A., K. Watanabe, et al. (1978). “Abnormal response of pancreatic glucagon to glycemic changes in diabetes mellitus.” J Clin Endocrinol Metab 46(3): 504-10.
Pearson and Lipman (1988) Improved tools for biological sequence comparison. Proc. Nat'l. Acad. Sci. USA 85: 2444
Petersen, K. F. and J. T. Sullivan (2001). “Effects of a novel glucagon receptor antagonist (Bay 27-9955) on glucagon-stimulated glucose production in humans.” Diabetologia 44(11): 2018-24.
Smith and Waterman (1981), Adv. Appl. Math. 2: 482
Sorensen, H., C. L. Brand, et al. (2006). “Immunoneutralization of endogenous glucagon reduces hepatic glucose output and improves long-term glycemic control in diabetic ob/ob mice.” Diabetes 55(10): 2843-8.
Sorensen, H., M. S. Winzell, et al. (2006). “Glucagon receptor knockout mice display increased insulin sensitivity and impaired beta-cell function.” Diabetes 55(12): 3463-9.
Strowski, M. Z., Z. Li, et al. (2004). “Small-molecule insulin mimetic reduces hyperglycemia and obesity in a nongenetic mouse model of type 2 diabetes.” Endocrinology 145(11): 5259-68.
Venkatesan N., Kim S. J., et al. (2003) Novel phosphoramidite building blocks in synthesis and applications toward modified oligonucleotides. Curr Med Chem 10(19): 1973-91.
Wahid M., Naveed, A. K., et al. (2007) “Hyperglucagonemia—a potent threat which can worsen the diabetes mellitus” Annals of King Edward Medical University Lahore Pakistan 13(4): 247-251.
Wincott F., DiRenzo A., et al. (1995) “Synthesis, deprotection, analysis and purification of RNA and ribozymes.” Nucleic Acids Res. 23(14):2677-84.

The features of the present invention disclosed in the specification, the claims and/or the drawings may both separately and in any combination thereof be material for realizing the invention in various forms thereof.

1-79. (canceled)

80. An L-nucleic acid molecule that binds glucagon selected from the group consisting of an L-nucleic acid molecule of type A, an L-nucleic acid molecule of type B and an L-nucleic acid molecule of type C, wherein
a) the L-nucleic acid molecule of type A comprises a central stretch of nucleotides
(SEQ ID NO: 173)
5′ Bn₁AAATGn₂GAn₃n₄GCTAKGn₅GGn₆n₇GGAATCTRRR 3′,
wherein n₁ is G or rG, n₂ is G or rG, n₃ is G or rG, n₄ is G or rG, n₅ is Y or rT, n₆ is A or rA, n₇ is A or rA; any of G, A, T, C, B, K, Y or R is a 2′-deoxyribonucleotide; and any of rG, rA of rT is a ribonucleotide;
b) the L-nucleic acid molecule of type B comprises
(SEQ ID NO: 197)
5′-AKGARn₁KGTTGSYAWAn₂RTTCGn₃TTGGAn₄TCn₅-′3,
(SEQ ID NO: 198)
5′-AGAAGGTTGGTAAGTTTCGGTTGGATCTG-′3,
(SEQ ID NO: 199)
5′-AGAAGGTCGGTAAGTTTCGGTAGGATCTG-′3,
(SEQ ID NO: 200)
5′-AGGAAGGTTGGTAAAGGTTCGGTTGGATTCA-′3,
(SEQ ID NO: 201)
5′-AGGAAAGGTTGGTAAGGTTCGGTTGGATTCA-′3
or
(SEQ ID NO: 202)
5′-AGGAAGGTTGGTAAGGTTCGGTTGGATTCA-′3,
wherein n₁ is A or rA, n₂ is G or rG, n₃ is C or rG, n₄ is T or rU, n₅ is A or rA; any of G, A, T, C, K, Y, S, W or R is a 2′-deoxyribonucleotide; and any of rG, rA or rU is a ribonucleotide; and
c) the L-nucleic acid molecule of type C comprises a nucleotide sequence selected from the group consisting of SEQ ID NO:83; SEQ ID NO:84, SEQ ID NO:85, SEQ ID NO: 86, SEQ ID NO:87, SEQ ID NO:97 and SEQ ID NO:102; nucleotide sequences comprising at least 85% identity thereto; or nucleotide sequences comprising at least 85% homology thereto.

81. The L-nucleic acid molecule according to claim 80, wherein the central stretch of nucleotides of the L-nucleic acid molecule of type A consists of 2′-deoxyribonucleotides or ribonucleotides.

82. The L-nucleic acid molecule according to claim 81, wherein the central stretch of nucleotides of the L-nucleic acid molecule of type A comprises
(SEQ ID NO: 179)
5′ GrGAAATGGGAGGGCTAGGTGGAAGGAATCTGAG 3′,
(SEQ ID NO: 180)
5′ GGAAATGrGGAGGCTAGGTGGAAGGAATCTGAG 3′,
(SEQ ID NO: 181)
5′ GGAAATGGGArGGGCTAGGTGGAAGGAATCTGAG 3′,
(SEQ ID NO: 182)
5′ GGAAATGGGAGrGGCTAGGTGGAAGGAATCTGAG 3′,
(SEQ ID NO: 183)
5′ GGAAATGGGAGGGCTAGGTGGrAAGGAATCTGAG 3′,
(SEQ ID NO: 184)
5′ GGAAATGGGAGGGCTAGGTGGArAGGAATCTGAG 3′;
(SEQ ID NO: 185)
5′ GGAAATGrGGAGGGCTAGGTGGrAAGGAATCTGAG 3′,
(SEQ ID NO: 186)
5′ GGAAATGGGAGGGCTAGGTGGrArAGGAATCTGAG 3′,
(SEQ ID NO: 187)
5′ GGAAATGrGGAGGGCTAGGTGGrArAGGAATCTGAG 3′,
(SEQ ID NO: 188)
5′ GGAAATGGGArGGGCTAGGTGGrArAGGAATCTGAG 3′,
(SEQ ID NO: 189)
5′ GrGAAATGrGGArGGGCTAGGTGGrArAGGAATCTGAG 3′,
(SEQ ID NO: 190)
5′ GrGAAATGrGGArGrGGCTAGGTGGrArAGGAATCTGAG 3′
or
(SEQ ID NO: 191)
5′ GrGAAATGrGGArGrGGCTAGGrTGGrArAGGAATCTGAG 3′,
wherein any of G, A, T or C is a 2′-deoxyribonucleotide, and any of rG, rA or rT is a ribonucleotide.

83. The L-nucleic acid molecule according to claim 80, wherein the central stretch of nucleotides of the L-nucleic acid molecule of type A consists of 2′-deoxyribonucleotides.

84. The L-nucleic acid molecule according to claim 80, wherein the Li-nucleic acid molecule of type A comprises in 5′→3′ direction
a) a first terminal stretch of nucleotides, the central stretch of nucleotides and a second terminal stretch of nucleotides: or
b) a second terminal stretch of nucleotides, the central stretch of nucleotides and a first terminal stretch of nucleotides,
wherein the first terminal stretch of nucleotides comprises one to seven nucleotides, and the second terminal stretch of nucleotides comprises one to seven nucleotides.

85. The L-nucleic acid molecule according to claim 84, wherein the first terminal stretch of nucleotides comprises 5′ Z₁Z₂Z₃Z₄Z₅Z₆V 3′ and the second terminal stretch of nucleotides comprises 5′ BZ₇Z₈Z₉Z₁₀Z₁₁Z₁₂ 3′, wherein Z₁ is G or absent, Z₂ is S or absent, Z₃ is V or absent, Z₄ is B or absent, Z₅ is B or absent, Z₆ is V or absent, Z₇ is B or absent, Z₈ is V or absent, Z₉ is V or absent, Z₁₀ is B or absent, Z₁₁ is S or absent, and Z₁₂ is C or absent.

86. The L-nucleic acid molecule according to claim 80, wherein the L-nucleic acid molecule of type A comprises SEQ ID NO:6 or SEQ ID NO:7; nucleic acids comprising at least 85% identity thereto; or nucleic acids comprising at least 85% homology thereto.

87. The L-nucleic acid molecule according to claim 80, wherein the L-nucleic acid molecule of type A comprises SEQ ID NO:23, SEQ ID NO:43, SEQ ID NO:47, SEQ ID NO:48, SEQ ID NO:91, SEQ ID NO:92, SEQ ID NO:158 or SEQ ID NO:159; nucleic acids comprising at least 85% identity thereto; or nucleic acids comprising at least 85% homology thereto.

88. The L-nucleic acid molecule according to claim 80, wherein the central stretch of nucleotides of the L-nucleic acid molecule of type B consists of 2′-deoxyribonucleotides or ribonucleotides.

89. The L-nucleic acid molecule according to claim 88, wherein the central stretch of nucleotides of the L-nucleic acid molecule of type B comprises
(SEQ ID NO: 204)
5′ AGGAArAGGTTGGTAAAGGTTCGGTTGGATTCA 3′,
(SEQ ID NO: 205)
5′ AGGAAAGGTTGGTAAArGGTTCGGTTGGATTCA 3′,
(SEQ ID NO: 206)
5′ AGGAAAGGTTGGTAAAGGTTCGGTTGGArUTCA 3′,
(SEQ ID NO: 207)
5′ AGGAArAGGTTGGTAAArGGTTCGGTTGGATTCA 3′,
(SEQ ID NO: 208)
5′ AGGAArAGGTTGGTAAAGGTTCGGTTGGArUTCG 3′,
(SEQ ID NO: 209)
5′ AGGAArAGGTTGGTAAAGGTTCGGTTGGArUTCA 3′,
(SEQ ID NO: 210)
5′ AGGAArAGGTTGGTAAArGGTTCGGTTGGArUTCA 3′
or
(SEQ ID NO: 211)
5′ AGGAArAGGTTGGTAAArGGTTCGrGTTGGArUTCrA 3′,
wherein any of G, A, T or C is a 2′-deoxyribonucleotide, and any of rG, rA or rU is a ribonucleotide.

90. The L-nucleic acid molecule according to claim 80, wherein the central stretch of nucleotides of the L-nucleic acid molecule of type B consists of 2′-deoxyribonucleotides.

91. The L-nucleic acid molecule according to claim 80, wherein the L-nucleic acid molecule of type B comprises in 5′→3′ direction
a) a first terminal stretch of nucleotides, the central stretch of nucleotides and a second terminal stretch of nucleotides; or
b) a second terminal stretch of nucleotides, the central stretch of nucleotides and a first terminal stretch of nucleotides,
wherein the first terminal stretch of nucleotides comprises three to nine nucleotides, and the second terminal stretch of nucleotides comprises three to ten nucleotides.

92. The L-nucleic acid molecule according to claim 91, wherein the first terminal stretch comprises 5′ Z₁Z₂Z₃Z₄Z₅Z₆SAK 3′ and the second terminal stretch comprises CKVZ₇Z₈Z₉Z₁₀Z₁₁Z₁₂ 3′, wherein Z₁ is C or absent, Z₂ is G or absent, Z₃ is R or absent, Z₄ is B or absent, Z₅ is B or absent, Z₆ is S or absent, Z₇ is S or absent, Z₈ is V or absent, Z₉ is V or absent, Z₁₀ is K or absent, Z₁₁ is M or absent, and Z₁₂ is S or absent.

93. The L-nucleic acid molecule according to claim 80, wherein the L-nucleic acid molecule of type B comprises SEQ ID NO:50, SEQ ID NO:54, SEQ II NO:58, SEQ ID NO:59, SEQ ID NO:88 or SEQ ID NO: 155; nucleic acids comprising at least 85% identity thereto; or nucleic acids comprising at least 85% homology thereto.

94. The L-nucleic acid molecule according to claim 80, wherein the L-nucleic acid molecule of type B comprises a nucleotide sequence selected from the group consisting of SEQ ID NO:71, SEQ ID NO:81, SEQ ID NO:82, SEQ ID NO:89, SEQ ID NO:90, SEQ ID NO: 156 and SEQ ID NO: 157; nucleic acids comprising at least 85% thereto; or nucleic acids comprising at least 85% homology thereto.

95. The L-nucleic acid molecule according to claim 80, wherein the L-nucleic acid molecule comprises an antagonist of glucagon activity.

96. The L-nucleic acid molecule according to claim 80, wherein the IL-nucleic acid molecule comprises a modification group.

97. The L-nucleic acid molecule according to claim 96, wherein the L-nucleic acid molecule comprising a modification group comprises an increased retention time in an animal or a human body as compared to an L-nucleic acid molecule not comprising the modification group; or a decreased excretion rate from an animal or a human body as compared to an L-nucleic acid molecule not comprising the modification group.

98. The L-nucleic acid molecule according to claim 96, wherein the modification group is a biodegradable modification or a non-biodegradable modifications.

99. The L-nucleic acid according to claim 96, wherein the modification group comprises linear polyethylene glycol, branched polyethylene glycol, hydroxyethyl starch, a peptide, a protein, a polysaccharide, a sterol, polyoxypropylene, polyoxyamidate or poly-(2-hydroxyethyl)-L-glutamine.

100. A pharmaceutical composition comprising the L-nucleic acid molecule as defined in claim 80 and optionally a further constituent, wherein the further constituent is selected from the group consisting of a pharmaceutically acceptable excipient, a pharmaceutically acceptable carrier and a pharmaceutically active agent.

101. A method of treating or preventing a disease or a disorder comprising administering to a subject suspected of comprising said disease or disorder, a therapeutically effective amount of the L-nucleic acid molecule according to claim 80.

102. The method of claim 101, wherein the disease or disorder is selected from the group consisting of diabetes, diabetic complication, diabetic condition and hyperglucagonemia.
